(19) United States
(12) Patent Application Publication
(10) Pub. No.: US 2025/0127880 A1
(43) Pub. Date: Apr. 24, 2025

(54) FUSION POLYPEPTIDE

(71) Applicant: VICTORIA LINK LIMITED, Kelburn, Wellington (NZ)

(72) Inventors: Davide Comoletti, Wellington (NZ); Lisa Connor, Wellington (NZ)

(21) Appl. No.: 18/685,637

(22) PCT Filed: Aug. 23, 2022
(86) PCT No.: PCT/IB2022/057859

§ 371 Date: Feb. 22, 2024

(30) Foreign Application Priority Data
Aug. 24, 2021 (AU) 2021902667
Jun. 3, 2022 (NZ) 788984

(51) Int. Cl. A61K 39/215; A61P 37/04; C07K 14/005

(52) U.S. Cl. CPC A61K 39/215; A61P 37/04; C07K 14/005; C07K 2319/00; C12N 2770/20022; C12N 2770/20034; C12N 2770/20071

(57) ABSTRACT

Disclosed herein are fusion polypeptides comprising at least one peptide domain from a severe acute respiratory syndrome coronavirus (SARS CoV-2) spike protein (S-protein), polynucleotides encoding such fusion polypeptides, methods of making such polypeptides and polynucleotides, pharmaceutical compositions and vaccines comprising such polypeptides or polynucleotides, and methods of using such polypeptides and/or polynucleotides for the treatment or prevention of SARS CoV-2 infection in a subject.

### FIELD OF THE INVENTION

This invention generally relates to fusion polypeptides comprising at least one peptide domain from a severe acute respiratory syndrome coronavirus (SARS CoV-2) spike protein (S-protein), a polynucleotide encoding such fusion polypeptides, methods of making such polypeptides and polynucleotides, pharmaceutical compositions and vaccines comprising such polypeptides or polynucleotides, and methods of using such polypeptides and/or polynucleotides for the treatment or prevention of SARS CoV-2 infection in a subject.

### BACKGROUND

The high transmissibility and pathogenicity of severe acute respiratory syndrome coronavirus (SARS CoV-2) is considered responsible for the recent Coronavirus disease 2019 or “Covid-19” pandemic (Hu et al. 2021).

For effective long-term prevention and control, vaccination is the most effective method available now and in the foreseeable future.

Although there are many different vaccine platforms against SARS-CoV-2 currently in development, there is no guarantee that any particular vaccine and/or group of vaccines will be effective, and/or remain effective in use. Additionally, once effective candidate molecules are identified, they must be produced at appropriate levels and in the appropriate form for therapeutic, particularly vaccine, use. Not all candidate molecules can be produced in a cost-effective manner, in sufficient quantities or at sufficient purity, to be useful therapeutically, including as vaccines.

Vaccines also vary in degrees of efficacy, and the need for additional new vaccines, particularly Coronavirus vaccines, is self-evident.

The scope of the current pandemic further suggests that many SARS CoV-2 variants may be anticipated in the future, due to mutation in multiple, unvaccinated human and/or animal populations.

Accordingly, there is a need now, and in the foreseeable future, for multiple new vaccines that can be used to treat or prevent SARS CoV-2 infection in humans and/or that can be used in the manufacture of medicaments for the treatment or prevention of SARS CoV-2 in humans.

It is an object of the present invention to provide at least one new therapeutic option for the treatment or prevention of SARS CoV-2 infection by providing at least one fusion polypeptide and/or at least one polynucleotide encoding such an fusion polypeptide, wherein the polypeptide and/or polynucleotide is effective in the treatment or prevention of SARS CoV-2 infection and/or wherein the polypeptide and/or polynucleotide is an effective SARS CoV-2 vaccine and/or wherein the polypeptide and/or polynucleotide at least provides the public with a useful choice.

In this specification where reference has been made to patent specifications, other external documents, or other sources of information, this is generally for the purpose of providing a context for discussing the features of the invention. Unless specifically stated otherwise, reference to such external documents is not to be construed as an admission that such documents, or such sources of information, in any jurisdiction, are prior art, or form part of the common general knowledge in the art.

### SUMMARY OF THE INVENTION

In one aspect the present invention relates to a fusion polypeptide or functional fragment or variant thereof comprising at least one SARS CoV-2 Spike protein (S-protein) receptor binding domain dimer (RBD dimer) fused to at least a portion of a SARS CoV-2 N-terminal domain (NTD) by a non-immunogenic amino acid linker, wherein the RBD dimer is located between the N-terminus of the fusion polypeptide and the portion of the NTD.

In another aspect the invention relates to a fusion polypeptide or functional fragment or variant thereof comprising in the following order from the N-terminus to the C-terminus, peptide domains a), b) and c), wherein
a) is a portion of the receptor binding domain (RBD) of the SARS CoV-2 S-protein,
b) is a portion of the receptor binding domain (RBD) of the SARS CoV-2 S-protein, and
c) is a portion of the N-terminal domain (NTD) of the SARS CoV-2 S-protein.

In another aspect the invention relates to a fusion polypeptide or a functional fragment or variant thereof comprising:
a. a first amino acid sequence comprising a first portion of the receptor binding domain (RBD) of a wild-type SARS CoV-2 S-protein,
b. a second amino acid sequence comprising a second portion of the receptor binding domain (RBD) of a wild-type SARS CoV-2 S-protein,
c. an amino acid sequence comprising at least a portion of the N-terminal domain (NTD) of a wild-type SARS CoV-2 protein,
d. a first non-immunogenic amino acid linker fusing a) to b), and
e. a second non-immunogenic amino acid linker fusing b) to c),
   wherein a) and b) are located between the N-terminus of the fusion polypeptide and c).

In one aspect the invention relates to a fusion polypeptide or functional fragment or variant thereof comprising in order from the N-terminal of the polypeptide: SEQ ID NO: 3, SEQ ID NO: 3, SEQ ID NO: 9.

In one aspect the invention relates to a fusion polypeptide consisting of a polypeptide or functional fragment or variant thereof comprising at least two copies of SEQ ID NO: 3 and at least one copy of SEQ ID NO: 9.

In another aspect the invention relates to a fusion polypeptide or functional fragment or variant thereof comprising at least 95% sequence identity to SEQ ID NO: 1.

In another aspect the invention relates to a fusion polypeptide or a functional fragment or variant thereof comprising the amino acid sequence of amino acid positions 1-210, 213-432 and 437 to 715 of SEQ ID NO: 1 or a functional fragment or variant thereof.

In another aspect the invention relates to a polynucleotide encoding a fusion polypeptide or functional fragment or variant thereof of the invention.

In another aspect the invention relates to an isolated polynucleotide or functional fragment or variant thereof comprising at least 70% nucleic acid sequence identity to a polynucleotide encoding a fusion polypeptide of the invention.

In another aspect the invention relates to a pharmaceutical composition comprising a fusion polypeptide or polynucleotide of the invention and a pharmaceutically acceptable carrier.

In another aspect the invention relates to a vaccine comprising a fusion polypeptide or polynucleotide of the invention and a pharmaceutically acceptable carrier.

Various embodiments of the different aspects of the invention as discussed above are also set out below in the detailed description of the invention, but the invention is not limited thereto. Other aspects of the invention may become apparent from the following description that is given by way of example only and with reference to the accompanying drawings.

### BRIEF DESCRIPTION OF THE DRAWINGS

The invention will now be described with reference to the figures in the accompanying drawings.

FIG. 1: Schematic representation of the purified fusion polypeptide Fc-608. Fc-608 comprises a tandem repeat (also termed an “RBD dimer” herein) of amino acid sequences from the RBD of the wild-type SARS CoV-2 S-protein. The RBD dimer is placed between the N-terminus of the polypeptide and an amino acid sequence from the wild-type SARS CoV-2 S-protein NTD. Non-immunogenic linkers within the fusion polypeptide may comprise any sequence of amino acid residues that is non-immunogenic. Exemplified herein are GSG and SGSG (SEQ ID NO: 5), but not limited thereto. GSG and SEQ ID NO: 5 are located between the individual RBDs and between the second RBD and the NTD respectively.

FIG. 2: Fc-608 expression and biochemical characterisation. A) Western blot of clonal selection of the expressed Fc-608 polypeptide. Fc-608 migrates at the expected molecular weight (MW) of the expressed polypeptides, confirming that the appropriate protein was being expressed in a sufficient amount to allow for downstream processing. Fc-608 was expressed in a HEK293, GnTI-cell line. The estimated molecular weight of the expressed protein is consistent with the expected glycosylated size of the protein, including the Fc moiety. B) Polyacrylamide gel electrophoresis of the purified Fc-608 polypeptide shows a single band of the appropriate MW mass. Light contamination from HRV-3Cpro enzyme used during the late stages of purification is observed (not shown). C) Matrix assisted laser desorption ionization-time of flight mass spectrometry (MALDI-TOF MS) characterization of the purified Fc-608 polypeptide showing the appropriate MW mass of the glycosylated purified protein (˜93 kDa). D) Size exclusion chromatography of the purified Fc-608 still containing the Fc moiety. Peak at 8.28 mL signals the excluded volume and the peak at 14.63 mL signals the pure protein.

FIG. 3A-B: A) Schematic representation of the domain organization of the non-expressed S-protein fusion polypeptide Fc-604 (SEQ ID NO: 15). B) Schematic representation of the domain organization of the non-expressed S-protein fusion polypeptide Fc-609 (SEQ ID NO: 17).

FIG. 4A-B: Immunisation with Fc608 evokes antibody response against SARS-CoV-2 RBD. A) C57BL/6 Mice were immunized by intramuscular injection twice, spaced 3 weeks apart (days 0 and 21). Groups of mice (n=5-10 mice/group) either received 50 μg of RBD monomer, RBD dimer, Fc608 or 6 μg of formalin inactivated SARS-CoV-2 virus, control unvaccinated mice were injected with PBS. All vaccines were prepared with 1:1 v/v with Addavax. Immune response to vaccine was assessed in spleen and blood at day 28. B) Anti-SARS CoV-2 RBD IgG titres were determined by conventional ELISA. Individual mice are indicated by symbols and line indicates geometric mean and geometric standard deviation of reciprocal serum endpoint titres. Statistical significance was determined by a One way ANOVA using a Sidak's multiple comparison test. ns>0.05, **p<0.01, ***P<0.001 and ****p<0.0001. Data show combination of two experiments.

FIG. 5A-B: Immunisation of mice with Fc608 evokes serum neutralizing antibodies against SARS-CoV-2 RBD. C57BL/6 mice (n=5-10/group/experiment) were immunized twice, three weeks apart with 50 μg of RBD monomer, RBD dimer, Fc608 or 6 μg of formalin inactivated SARS-CoV-2 virus. Control unvaccinated mice were injected with PBS. All vaccines were prepared with 1:1 v/v with Addavax. Serum taken from mice at 28 days was assessed for virus neutralization activity. A) Surrogate neutralization assay (sVNT) is represented as percent ACE2 binding to RBD in presence of serum (diluted 1:10). B) Replication competent SARS-CoV-2 neutralization assay (rcVNT) shown as the 50% inhibitory dilution titer (ID₅₀), calculated as the reciprocal of the dilution that reduced cytopathic effect by 50%. Data represents a combination of two experiments. Each symbol represents an individual serum sample, and the line represents mean and standard deviation (a) or the geometric mean and geometric standard deviation (b) of the group. Statistical analysis was performed using a One way ANOVA and Sidak's multiple comparison test ****p<0.0001.

FIG. 6: Immunisation of mice with Fc608 evokes RBD-specific IFNγ producing T cell response. C57BL/6 mice (n=5-10/group/experiment) were immunized twice, three weeks apart with 50 μg of RBD monomer, RBD dimer, Fc608 or 6 μg of formalin inactivated SARS-CoV-2 virus. Control unvaccinated mice were injected with PBS. All vaccines were prepared with 1:1 v/v with Addavax. Splenocytes taken from mice at 28 days were stimulated with media alone, or peptide pools containing overlapping 15mer peptides derived from either the entire S1 domain or RBD of SARS-CoV-2 spike protein and assessed for IFNγ production in an ELISpot assay. Bar graph shows the number of IFNγ secreting cells per million splenocytes. Each symbol represents an individual serum sample, and the line represents mean and standard deviation (a) or the geometric mean and geometric standard deviation (b) of the group. Statistical analysis was performed using a two way ANOVA and Tukey's multiple comparison test **p<0.01,****p<0.0001.

FIG. 7A-C: a) mRNA-Fc608 encodes a fusion protein of a receptor binding domain (RBD) tandem dimer with the N-terminal domain of the Spike protein fused to the C-terminus (RBD-RBD-NTD). b) Schematic map of the plasmid encoding the RBD-RBD-NTD to be used as mRNA vaccine. Main domains and untranslated regions are labelled, c) Western blot confirms the expression of a protein product at the expected size ˜90 kDa in HEK293T cells.

FIG. 8: mRNA vaccination immunogenicity study design.

FIG. 9A-C: A 608 mRNA elicits humoral and T cell immunity in mice. a) a-RBD binding antibodies; b) SARS CoV-2 neutralizing antibodies; c) RBD and S1 specific T cell response.

FIG. 10A-B: Immunization with Fc608 provides protection equivalent to that seen in convalescent K-18 mice. a) body weight changes compared to mortality; b) survival over time.

FIG. 11: Immunization with Fc608 induces long-lived antibodies equivalent to that seen in convalescent mice.

FIG. 12A-B: Immunity generated by Fc628 (delta version) is comparable to Fc608 (Wuhan version). a) IFNγ-producing T cells specific for S1; b) RBD binding antibodies FIG. 13: Immunization with Fc628 provides cross protection against SARS-CoV-2 variants of concern.

FIG. 14A-B: Incorporation of the NTD enhances immunogenicity of the RBD subunit vaccine. a) expansion of S1 specific T cells; b) RBD-specific T cell response; c) Tfh CD4+ cells detected in mice vaccinated with Fc628

FIG. 15A-B: Incorporation of the NTD improves the antibody response to the RBD. a) anti-RBD IgG antibodies; b) affinity compared to titers in mice immunized with RBD dimer.

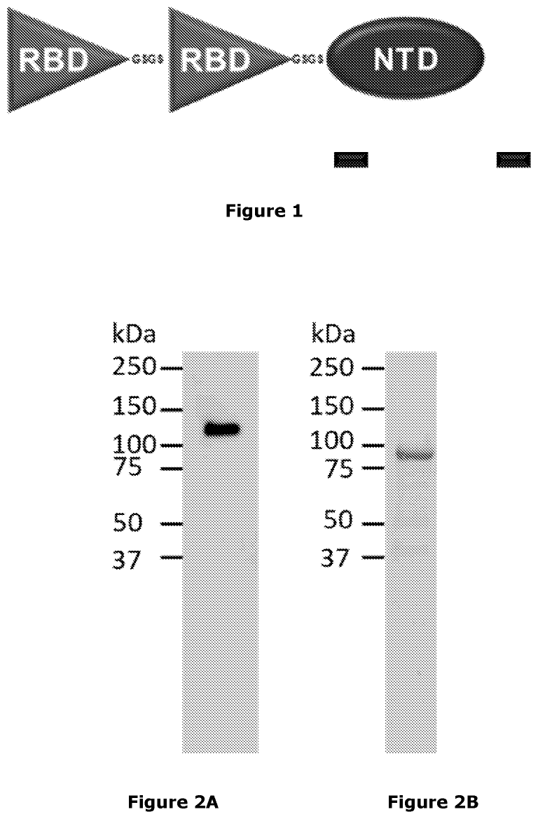

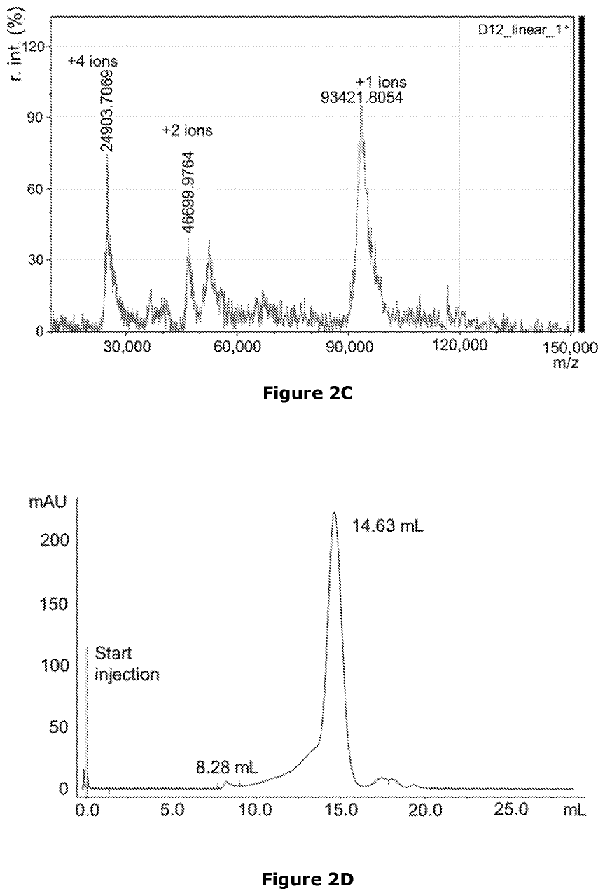

### DETAILED DESCRIPTION OF THE INVENTION

### Definitions

The term “comprising” as used in this specification and claims means “consisting at least in part of”; that is to say when interpreting statements in this specification and claims which include “comprising”, the features prefaced by this term in each statement all need to be present but other features can also be present. Related terms such as “comprise” and “comprised” are to be interpreted in similar manner.

The term “consisting essentially of” as used herein means the specified materials or steps and those that do not materially affect the basic and novel characteristic(s) of the claimed invention.

The term “consisting of” as used herein means the specified materials or steps of the claimed invention, excluding any element, step, or ingredient not specified in the claim.

The term “polynucleotide(s),” as used herein, refers in its broadest sense to a single or double-stranded deoxyribonucleotide or ribonucleotide polymer of any length, and includes as non-limiting examples, coding and non-coding sequences of a gene, sense and antisense sequences, exons, introns, genomic DNA, cDNA, pre-mRNA, mRNA, rRNA, siRNA, miRNA, tRNA, ribozymes, recombinant polynucleotides, isolated and purified naturally occurring DNA or RNA sequences, synthetic RNA and DNA sequences, nucleic acid probes, primers, fragments, genetic constructs, vectors and modified polynucleotides. Reference to nucleic acids, nucleic acid molecules, nucleotide sequences and polynucleotide sequences is to be similarly understood.

In some embodiments the polynucleotides described herein are isolated.

Nucleic acids as contemplated herein may be, or include (but not limited thereto), deoxyribonucleic acids (DNAs), ribonucleic acids (RNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization), threose nucleic acids (TNAs), ethylene nucleic acids (ENA), cyclohexenyl nucleic acids (CeNA), glycol nucleic acids (GNAs), or chimeras or combinations thereof.

In some embodiments, a nucleic acid or polynucleotide as described herein is a messenger RNA (mRNA). The term “messenger RNA” (mRNA) as used herein refers to any polynucleotide that encodes a polypeptide of interest, such as one described herein, and that can be translated in vitro, in vivo, ex vivo or in situ to produce the polypeptide.

The encoded polypeptide may be a naturally occurring, non-naturally occurring, or modified polymer of amino acids. In a preferred embodiment, the encoded polypeptide is a non-naturally occurring polypeptide. As used herein unless specifically indicated otherwise, DNA polynucleotide sequences described herein will recite thymine (T) whereas RNA polynucleotide sequences the thymine is replaced with uracil (U). Accordingly, the skilled person recognizes that any of the polynucleotides encoded by a specifically identified DNA (i.e., by a SEQ ID NO:), is considered to comprise the corresponding RNA (e.g., mRNA) sequence where each thymine the DNA sequence is substituted with uracil (i.e., T>U substitution).

The person skilled in the art also appreciates that an mRNA that can be translated into a polypeptide of interest will also include some or all of the following features: a 5′ cap, a 5′ untranslated region (UTR), at least one coding region, a 3′ UTR, and a poly-A tail.

Polynucleotides described herein may function as mRNA and are distinguished from wild-type mRNA in their functional and/or structural design features, which provide a basis for new and highly effective SARS CoV-2 therapeutics.

The term “open reading frame” means a continuous stretch of DNA beginning with a start codon (e.g., methionine (ATG)), and ending with a stop codon (e.g., TAA, TAG or TGA). An open reading frame encodes a polypeptide.

The term “3′ untranslated region” (3′UTR) is used herein as understood by the skilled person and refers to a region of an mRNA that is directly downstream (i.e., 3′) from the stop codon (i.e., the codon of an mRNA transcript that signals a termination of translation). The 3′UTR does not comprise an open reading frame and/or is not translated into a polypeptide.

The term “5′ untranslated region” (5′UTR) is used herein as understood by the skilled person and refers to a region of an mRNA that is directly upstream (i.e., 5′) from the start codon (i.e., the first codon of an mRNA transcript translated by a ribosome). The 5′UTR does not comprise an open reading frame and/or is not translated into a polypeptide.

As used herein, the term “polyA tail” means a region of mRNA that is downstream (i.e., 3′) from the 3′ UTR and that contains multiple, consecutive adenosine monophosphates (A residues). As is appreciated in the art, the function of the poly(A) tail is to protect an mRNA from enzymatic degradation as well as to facilitate both transcription termination and mRNA export from the nucleus. The number of consecutive A residues in a “poly A tail” may vary; e.g., from 10 to 300. By way of example only, a polyA tail may contain 10, 20, 30, 40, 50, 60, 70, 80, 90, 100, 110, 120, 130, 140, 150, 160, 170, 180, 190, 200, 210, 220, 230, 240, 250, 260, 270, 280, 290 or 300 A residues.

The term “transcription unit” (TU) as used herein refers to a polynucleotide comprising a sequence of nucleotides that code for a single RNA molecule including all the nucleotide sequences necessary for transcription of the single RNA molecule, including a promoter, an RNA-coding sequence, and a terminator, but not limited thereto.

The term “vector” as used herein refers to any type of polynucleotide molecule that may be used to manipulate genetic material so that it can be amplified, replicated, manipulated, partially replicated, modified and/or expressed, but not limited thereto. In some embodiments a vector may be used to transport a polynucleotide comprised in that vector into a cell or organism. In some embodiments a vector is selected from the group consisting of plasmids, bacterial artificial chromosomes (BACs), P1-derived artificial chromosomes (PACs), yeast artificial chromosomes (YACs), bacteriophage, phagemids, and cosmids. In a preferred embodiment, a vector is a plasmid.

The term “coding region” or “open reading frame” (ORF) refers to the sense strand of a genomic DNA sequence or a cDNA sequence that is capable of producing a transcription product and/or a polypeptide under the control of appropriate regulatory sequences. The coding sequence is identified by the presence of a 5′ translation start codon and a 3′ translation stop codon. When inserted into a genetic construct or an expression cassette, a “coding sequence” is capable of being expressed when it is operably linked to promoter and terminator sequences and/or other regulatory elements.

“Operably-linked” means that the sequence to be expressed is placed under the control of regulatory elements.

“Regulatory elements” as used herein refers to any nucleic acid sequence element that controls or influences the expression of a polynucleotide insert from a vector, genetic construct or expression cassette and includes promoters, transcription control sequences, translation control sequences, origins of replication, tissue-specific regulatory elements, temporal regulatory elements, enhancers, polyadenylation signals, repressors, and terminators. Regulatory elements can be “homologous” or “heterologous” to the polynucleotide insert to be expressed from a genetic construct, expression cassette or vector as described herein. When a genetic construct, expression cassette or vector as described herein is present in a cell, a regulatory element can be “endogenous”, “exogenous”, “naturally occurring” and/or “non-naturally occurring” with respect to cell.

The term “noncoding region” refers to untranslated sequences that are upstream of the translational start site and downstream of the translational stop site. These sequences are also referred to respectively as the 5′ UTR and the 3′ UTR. These regions include elements required for transcription initiation and termination and for regulation of translation efficiency.

Terminators are sequences, which terminate transcription, and are found in the 3′ untranslated ends of genes downstream of the translated sequence. Terminators are important determinants of mRNA stability and in some cases have been found to have spatial regulatory functions.

The term “promoter” refers to non-transcribed cis-regulatory elements upstream of the coding region that regulate the transcription of a polynucleotide sequence. Promoters comprise cis-initiator elements which specify the transcription initiation site and conserved boxes. In one non-limiting example, bacterial promoters may comprise a “Pribnow box” (also known as the −10 region), and other motifs that are bound by transcription factors and promote transcription. Promoters can be homologous or heterologous with respect to polynucleotide sequence to be expressed. When the polynucleotide sequence is to be expressed in a cell, a promoter may be an endogenous or exogenous promoter. Promoters can be constitutive promoters, inducible promoters or regulatable promoters as known in the art.

The term “polypeptide(s),” as used herein, is used in a broad sense to include naturally occurring polypeptides, artificial polypeptides, synthetic polypeptides, gene products, homologs, orthologs, paralogs, variants, fragments, and other equivalents, as well as analogs of such as would be appreciated by a skilled person in the art. A polypeptide may be a single molecule or may part of a molecular complex. Such complexes include, but are not limited to, dimers, trimers, tetramers, hexamers, and the like. A polypeptide can comprise a single chain of amino acids (i.e., a single polypeptide), or, in the case of a molecular complex, multiple chains of amino acids (multiple polypeptides). Frequently, molecular complexes comprising multiple polypeptides comprise disulfide bridges or linkages between certain amino acid residues. As used herein, the term “polypeptide” also refers to polymers of amino acid residues comprising at least one modified amino acid residue, including as a non-limiting example, an artificial chemical analogue of a corresponding naturally occurring amino acid.

“Naturally occurring” as used herein with reference to a polypeptide or polynucleotide refers to a polynucleotide or polypeptide sequence having a primary nucleic acid or amino acid sequence that is found in nature. A synthetic polynucleotide or polypeptide sequence that is identical to a wild-type polynucleotide sequence is, for the purposes of this disclosure, considered a naturally occurring sequence. What is important for a naturally occurring polynucleotide or polypeptide sequence is that the actual sequence of nucleotide bases or amino acid residues that make up the polynucleotide or polypeptide respectively, is as found or as known from nature.

The term “wild-type” is used here as generally understood in the art. For example, a wild-type polynucleotide sequence is a naturally occurring polynucleotide sequence, but not limited thereto. A naturally occurring polynucleotide sequence also refers to variant polynucleotide sequences as found in nature that differ from wild-type. For example, allelic variants and naturally occurring recombinant polynucleotide sequences due to hybridization or horizontal gene transfer, but not limited thereto.

Specifically contemplated herein, the wild-type SARS CoV-2 S-protein is the full-length amino acid sequence (aa 1-1273) having UniProt Accession No: PODTC2.

Specifically contemplated herein, the nucleic acid sequence encoding the wild-type SARS CoV-2 S-protein is the nucleic acid sequence having GenBank Accession No.: YP_009724390.1.

“Non-naturally occurring” as used herein with reference to a polypeptide or polynucleotide refers to a polynucleotide or polypeptide having a primary nucleic acid or amino acid sequence that is not found in nature. Such peptides are also called “artificial polypeptides” (and grammatical variations thereof) herein.

Examples of non-naturally occurring polynucleotide and polypeptide sequences include artificially produced mutant and variant polynucleotide and polypeptide sequences, made for example by point mutation, insertion, or deletion, domain rearrangement, but not limited thereto. Non-naturally occurring polynucleotide and polypeptide sequences also include chemically evolved sequences. What is important for a non-naturally occurring polynucleotide or polypeptide sequence as described herein is that the actual sequence of nucleotide bases or amino acid residues that make up the polynucleotide or polypeptide respectively, are not found in or known from nature.

The term “fused” as used herein with reference to polypeptides and portions of polypeptides that are “fused” together (including other grammatical variations) means that the amino acid sequences are covalently joined to each other by peptide bonds.

The “fusion polypeptides” disclosed in the present application are artificial polypeptides, i.e., the fusion polypeptides disclosed herein are non-naturally occurring.

In some embodiments the fusion polypeptides described herein are immunogenic or antigenic. The terms “immunogenic” and “antigenic” are used interchangeably herein and are taken to mean the same thing.

“Homologous” as used herein with reference to a polynucleotide or polypeptide or part thereof means a polynucleotide or polypeptide or part thereof that is a naturally occurring polynucleotide or polypeptide or part thereof.

“Heterologous” as used herein with reference to a polynucleotide or polypeptide or part thereof means a polynucleotide or polypeptide or part thereof that is a non-naturally occurring polynucleotide or polypeptide or part thereof.

A homologous polynucleotide or part thereof may be operably linked to one or more different polynucleotides or parts thereof to form a single polynucleotide that can be expressed or translated in a cell to form a polypeptide of interest, preferably an antigenic polypeptide. In some embodiments the different polynucleotides or parts thereof are homologous polynucleotides or parts thereof. In some embodiments the different polynucleotides or parts thereof are heterologous polynucleotides or parts thereof.

Likewise, a heterologous polypeptide or part thereof may be fused to one or more different polypeptides or parts thereof to form a single polypeptide of interest, preferably an antigenic polypeptide. In some embodiments the different polypeptides or parts thereof are homologous polypeptides or parts thereof. In some embodiments the different polypeptides or parts thereof are heterologous polypeptides or parts thereof.

The term “functional variant or fragment thereof” of a polypeptide refers to a subsequence of the polypeptide that performs a function that is required for the biological activity or binding of that polypeptide and/or provides the three-dimensional structure of the polypeptide. The term may refer to a polypeptide, an aggregate of a polypeptide such as a dimer or other multimer, a fusion polypeptide, a polypeptide fragment, a polypeptide variant, or functional polypeptide derivative thereof performs the polypeptide activity.

“Isolated” as used herein with reference to polynucleotide or polypeptide sequences describes a sequence that has been removed from its natural cellular environment or from a cellular environment in which it was synthesized or expressed. An isolated molecule may be obtained by any method or combination of methods as known and used in the art, including biochemical, recombinant, and synthetic techniques. The polynucleotide or polypeptide sequences may be prepared by at least one purification step.

In some embodiments a fusion polypeptide as described herein is isolated. In some embodiments a polynucleotide as described herein is isolated.

As used herein, the term “variant” refers to polynucleotide or polypeptide sequences different from the specifically identified sequences, wherein one or more nucleotides or amino acid residues is deleted, substituted, or added. Variants may be naturally occurring allelic variants, or non-naturally occurring variants. Variants may be from the same or from other species and may encompass homologues, paralogues, and orthologues. In certain embodiments, variants of the polynucleotides and polypeptides described herein have biological activities that are the same, similar, or substantially similar to those of a corresponding wild-type molecule, i.e., the naturally occurring polypeptides or polynucleotides. In certain embodiments the similarities are similar activity and/or binding specificity.

In certain embodiments, variants of the polynucleotides and polypeptides described herein have biological activities that differ from their corresponding wild-type molecules. In certain embodiments the differences are altered activity and/or binding specificity.

The term “variant” with reference to polynucleotides and polypeptides encompasses all forms of polynucleotides and polypeptides as defined herein.

### Polynucleotide Variants

Variant polynucleotide sequences preferably exhibit at least 50%, at least 60%, preferably at least 70%, preferably at least 71%, preferably at least 72%, preferably at least 73%, preferably at least 74%, preferably at least 75%, preferably at least 76%, preferably at least 77%, preferably at least 78%, preferably at least 79%, preferably at least 80%, preferably at least 81%, preferably at least 82%, preferably at least 83%, preferably at least 84%, preferably at least 85%, preferably at least 86%, preferably at least 87%, preferably at least 88%, preferably at least 89%, preferably at least 90%, preferably at least 91%, preferably at least 92%, preferably at least 93%, preferably at least 94%, preferably at least 95%, preferably at least 96%, preferably at least 97%, preferably at least 98%, and preferably at least 99% identity to a sequence of the present invention. Identity is found over a comparison window of at least 8 nucleotide positions, preferably at least 10 nucleotide positions, preferably at least 15 nucleotide positions, preferably at least 20 nucleotide positions, preferably at least 27 nucleotide positions, preferably at least 40 nucleotide positions, preferably at least 50 nucleotide positions, preferably at least 60 nucleotide positions, preferably at least 70 nucleotide positions, preferably at least 80 nucleotide positions, preferably over the entire length of a polynucleotide as described herein.

Polynucleotide variants also encompass those which exhibit a similarity to one or more of the specifically identified sequences that is likely to preserve the functional equivalence of those sequences, and which could not reasonably be expected to have occurred by random chance.

Polynucleotide sequence identity and similarity can be determined readily by those of skill in the art.

Variant polynucleotides also encompass polynucleotides that differ from the polynucleotide sequences described herein but that, due to the degeneracy of the genetic code, encode a polypeptide having similar activity to a polypeptide encoded by a polynucleotide of the present invention. A sequence alteration that does not change the amino acid sequence of the polypeptide is a “silent variation”. Except for ATG (methionine) and TGG (tryptophan), other codons for the same amino acid may be changed by art recognized techniques, e.g., to optimize codon expression in a particular host organism.

Polynucleotide sequence alterations resulting in conservative substitutions of one or several amino acids in the encoded polypeptide sequence without significantly altering its biological activity are also included in the invention. A skilled artisan will be aware of methods for making phenotypically silent amino acid substitutions (see, e.g., Bowie et al., 1990, Science 247, 1306).

In the context of the present description, a “functional variant or fragment thereof” of a polynucleotide is one that comprises additions, substitutions and/or deletions in the nucleotide residues that code for non-essential amino acid residues, and/or of non-essential amino acid sequences (e.g., of SEQ ID NO: 1), where “non-essential” means amino acid residues or sequences that do not affect the antigenicity and/or immunogenicity and/or ability of the fusion polypeptide to be expressed in sufficient amounts for downstream processing.

A person skilled in the art appreciates that a sufficient amount for downstream processing refers to a sufficient amount of the polypeptide being expressed to allow for ultimate production at a commercial level.

In some embodiments, a functional variant of a fusion polypeptide as described herein is a fusion polypeptide comprising SEQ ID NO: 1 wherein the amino acid residues at positions 1, 211-213 and 433-436 can be any amino acid residues such that the functional variant has at least about the same immunogenicity and/or antigenicity and/or ability to be expressed in sufficient amounts for downstream processing as SEQ ID NO: 1.

In some embodiments a functional variant of a polynucleotide as described herein is a polynucleotide comprising a nucleic acid sequence encoding at least the first portion of an RBD and at least a second portion of an RBD positioned between the 5′ end of the polynucleotide and a nucleic acid sequence encoding at least a portion of an NTD, wherein a polypeptide expressed from the polynucleotide functional variant has at least about the same immunogenicity and/or antigenicity and/or ability to be expressed in sufficient amounts for downstream processing.

In some embodiments, polynucleotides as described herein may be codon optimized. Codon optimization methods are known in the art and may be used as known to the skilled worker and as described herein. For example, codon optimization may be used to match codon frequencies in target and host organisms, ensuring proper polynucleotide folding; to increase mRNA stability (by modification of GC content), to reduce secondary structures; to minimize tandem repeat codons or base runs that might impact polynucleotide construction or expression; to customize control regions (transcriptional and translational); to insert or remove protein trafficking sequences; to add or remove post translation modification sites such as glycosylation sites; to add, remove or rearrange protein domains and restriction sites, to modify ribosome binding sites, to alter mRNA degradation sites; to adjust rates of protein translation to mediate accurate folding; and/or to address issues related to problematic secondary structure of the polynucleotide per se. The inventors believe that codon optimization tools, algorithms and services are known in the art—non-limiting examples include services from GeneArt (Life Technologies), DNA2.0 (Menlo Park Calif.) and/or proprietary methods. In some embodiments, the open reading frame (ORF) sequence is optimized using optimization algorithms.

By way of non-limiting examples, a codon optimized sequence as contemplated herein may share less than 95% sequence identity, 90% sequence identity, 85% sequence identity, 80% sequence identity, 75% sequence identity, or less than 70% sequence identity to a naturally-occurring or wild-type sequence polynucleotide sequence. In one example the naturally-occurring or wild-type mRNA sequence encodes an immunogenic or antigenic polypeptide, but not limited thereto. A codon-optimized polynucleotide sequence as described herein may share between 65% and 85% sequence identity to a naturally-occurring sequence or a wild-type polynucleotide sequence, including any combination of % identities in this range (e.g., 68%-80%; 70%-76%; 65% to 80% but not limited thereto).

In some embodiments a codon-optimized polynucleotide as described herein is a codon optimised RNA. In one embodiment the codon optimised RNA is an mRNA encoding an immunogenic or antigenic polypeptide as described herein. In some embodiments, the codon optimised RNA may comprise an increased GC content which can be designed to influence the stability of the RNA. A person skilled in the art appreciates that RNA having an increased amount of guanine (G) and/or cytosine (C) residues may be functionally more stable than nucleic acids containing a large amount of adenine (A) and thymine (T) or uracil (U) nucleotides. WO02/098443 (the entirety of which is hereby incorporated by reference) discloses mRNAs comprising sequence modifications that confer greater stability on the translated region of the polynucleotide. The modifications leverage the degeneracy of the genetic code, substituting existing codons for alternative codons that promote greater RNA stability without a resulting amino acid change. The modifications may only be applied to the open reading frames of the translated RNA.

### Polypeptide Variants

The term “variant” with reference to polypeptides also encompasses naturally occurring, recombinantly and synthetically produced polypeptides. Variant polypeptide sequences preferably exhibit at least 35%, preferably at least 40%, preferably at least 50%, preferably at least 60%, preferably at least 70%, preferably at least 71%, preferably at least 72%, preferably at least 73%, preferably at least 74%, preferably at least 75%, preferably at least 76%, preferably at least 77%, preferably at least 78%, preferably at least 79%, preferably at least 80%, preferably at least 81%, preferably at least 82%, preferably at least 83%, preferably at least 84%, preferably at least 85%, preferably at least 86%, preferably at least 87%, preferably at least 88%, preferably at least 89%, preferably at least 90%, preferably at least 91%, preferably at least 92%, preferably at least 93%, preferably at least 94%, preferably at least 95%, preferably at least 96%, preferably at least 97%, preferably at least 98%, and preferably at least 99% identity to a sequence of the present invention. Identity is found over a comparison window of at least 2 amino acid positions, preferably at least 3 amino acid positions, preferably at least 4 amino acid positions, preferably at least 5 amino acid positions, preferably at least 7 amino acid positions, preferably at least 10 amino acid positions, preferably at least 15 amino acid positions, preferably at least 20 amino acid positions, preferably over the entire length of a polypeptide as described herein.

Polypeptide variants also encompass those which exhibit a similarity to one or more of the specifically identified sequences that is likely to preserve the functional equivalence of those sequences, and which could not reasonably be expected to have occurred by random chance.

Polypeptide sequence identity and similarity can be determined readily by those of skill in the art.

A variant polypeptide includes a polypeptide wherein the amino acid sequence differs from a polypeptide herein by one or more conservative amino acid or non-conservative substitutions, deletions, additions, or insertions which do not affect the biological activity of the peptide.

Conservative substitutions typically include the substitution of one amino acid for another with similar characteristics, e.g., substitutions within the following groups: valine, glycine; glycine, alanine; valine, isoleucine, leucine; aspartic acid, glutamic acid; asparagine, glutamine; serine, threonine; lysine, arginine; and phenylalanine, tyrosine.

Analysis of evolved biological sequences has shown that not all sequence changes are equally likely, reflecting at least in part the differences in conservative versus non-conservative substitutions at a biological level. For example, certain amino acid substitutions may occur frequently, whereas others are very rare. Evolutionary changes or substitutions in amino acid residues can be modelled by a scoring matrix also referred to as a substitution matrix. Such matrices are used in bioinformatics analysis to identify relationships between sequences and are known to the skilled worker.

Other variants include peptides with modifications which influence peptide stability. Such analogs may contain, for example, one or more non-peptide bonds (which replace the peptide bonds) in the peptide sequence. Also included are analogs that include residues other than naturally occurring L-amino acids, e.g., D-amino acids or non-naturally occurring synthetic amino acids, e.g., beta or gamma amino acids and cyclic analogs.

Substitutions, deletions, additions, or insertions may be made by mutagenesis methods known in the art. A skilled worker will be aware of methods for making phenotypically silent amino acid substitutions. See for example Bowie et al., 1990, Science 247, 1306.

A polypeptide as used herein can also refer to a polypeptide that has been modified during or after synthesis, for example, by biotinylation, benzylation, glycosylation, phosphorylation, amidation, by derivatization using blocking/protecting groups and the like. Such modifications may increase stability or activity of the polypeptide.

In the context of the present description, a “functional variant or fragment thereof” of a fusion polypeptide is one that comprises additions, substitutions and/or deletions of non-essential amino acid residues, and/or of non-essential amino acid sequences (e.g., of SEQ ID NO: 1), where “non-essential” means amino acid residues or sequences that do not affect the antigenicity and/or immunogenicity and/or ability of the fusion polypeptide to be expressed in sufficient amounts for downstream processing.

A person skilled in the art appreciates that sufficient amounts for downstream processing refers to sufficient amounts for ultimate production at a commercial level.

In some embodiments, a functional variant of a fusion polypeptide as described herein is a fusion polypeptide comprising SEQ ID NO: 1 wherein the amino acid residues at positions 1, 211-213 and 433-436 can be any amino acid residues such that the functional variant has at least about the same immunogenicity and/or antigenicity and/or ability to be expressed in sufficient amounts for downstream processing as SEQ ID NO: 1.

In some embodiments a functional variant of a fusion polypeptide as described herein is a fusion polypeptide comprising at least first portion of an RBD and at least a second portion of an RBD positioned between the N-terminus of the polypeptide and at least a portion of an NTD, wherein the functional variant has at least about the same immunogenicity and/or antigenicity and/or ability to be expressed in sufficient amounts for downstream processing.

Without wishing to be bound by theory, the inventors believe that certain amino acid sequences of the fusion polypeptides described herein as non-immunogenic linkers, can be substituted for certain other non-immunogenic amino acid linkers without effecting the antigenicity and/or immunogenicity and/or expression levels of the fusion polypeptides.

These “non-immunogenic amino acid linkers” as described herein comprise sequences of amino acid residues that do not provoke an immune response in a subject, i.e., that are non-immunogenic. The fusion polypeptides described herein comprise non-immunogenic linkers that are positioned between RBD and RBD amino acid sequences, or between RBD and NTD amino acid sequences as described herein. The amino acid sequence of the non-immunogenic amino acid linker may any amino acid sequence as known in the art to the skilled worker. Selection of particular non-immunogenic linkers for use in a fusion polypeptide as described herein is believed to be within the skill of those in the art, and the invention disclosed herein is not limited by such a selection.

Rather what is important in a fusion polypeptide as described herein is the positioning of the RBD and NTD peptide domains of the wild-type SARS CoV-2 S-protein in non-naturally occurring and opposite positions to each other relative to their natural positions within the polypeptide.

The skilled worker appreciates that as long as the relative spacing and non-immunogenic character of the linker is maintained, slight variations in amino acid composition or length of the linker will be well tolerated and are contemplated herein.

As used herein, the phrases “a receptor binding domain (RBD) of a wild-type SARS CoV-2 S-protein” and “at least a portion of a receptor binding domain (RBD) of a wild-type SARS CoV-2 S-protein”(and grammatical variations thereof) mean the amino acid sequence of the RBD or, or of the at least a portion of the RBD, respectively.

As used herein the terms “an RBD” and “at least a portion of an RBD” are abbreviations of the terms “an RBD of the wild-type SARS CoV-2 S-protein” and “at least a portion of an RBD of a wild-type SARS CoV-2 S-protein” respectively.

As used herein, the phrases “an N-terminal domain (NTD) of a wild-type SARS CoV-2 S-protein” and “at least a portion of an N-terminal domain (NTD) of a wild-type SARS CoV-2 S-protein” (and grammatical variations thereof) mean the amino acid sequence of the RBD, or of the at least a portion of the RBD, respectively.

As used herein the terms “an NTD” and “at least a portion of an NTD” are abbreviations of the terms “an NTD of the wild-type SARS CoV-2 S-protein” and “at least a portion of an NTD of a wild-type SARS CoV-2 S-protein” respectively.

An “RBD dimer” as used herein is an amino acid sequence that comprises two copies of an amino acid sequence from the receptor binding domain (RBD) of the SARS CoV-2 S-protein (SEQ ID NO: 19) positioned adjacent each other as a tandem repeat. In an RBD dimer as described herein, the two copies of the RBD amino acid sequence do not need to be directly adjacent but may be linked by from 1 to 10 amino acid residues as described herein. The number of amino acid residues that may be comprised in the linker may vary. What is important is that a fusion polypeptide as described herein that comprises an RBD dimer, is expressed, as described herein, as or as part of an immunogenic or antigenic fusion polypeptide. Such expression is in sufficient amounts for downstream processing.

As used herein the term “tandem repeat” refers to a pattern that occurs in a polypeptide or polynucleotide where one or more residues is repeated, and the repetitions are positioned directly adjacent or substantially directly adjacent to each other. Tandem repeats can be formed by protein domains where the amino acid sequence of a protein domain is repeated within the amino acid primary structure.

In one embodiment an RBD dimer as described herein is a polypeptide tandem repeat of SEQ ID NO: 3.

As used herein the term “N-terminus” of a polypeptide means the last residue of the polypeptide chain that has an exposed amino terminus.

As used herein the “C-terminus” of a polypeptide means the last residue of a polypeptide with an exposed carboxy terminus.

As used herein the term “protease binding site” refers to a sequence of amino acid residues that is recognized and cleaved by a protease.

The term “therapeutically effective amount” (or “effective amount”) refers to an amount sufficient to effect beneficial or desired results, including clinical results, but not limited thereto. A therapeutically effective amount of the antigenic polypeptide or polynucleotide described herein can be administered in one or more administrations separated by an appropriate amount of time (where required). The therapeutically effective amount of polypeptide or polynucleotide be administered to a subject depends on, for example, the mode of administration, nature and dosage of any co-administered compounds, and characteristics of the subject, such as general health, other diseases, age, sex, genotype, body weight and tolerance to drugs. A person skilled in the art will be able to determine appropriate dosages having regard to these any other relevant factors.

The term “subject” refers to a human or a non-human animal, preferably a vertebrate that is a mammal. Non-human mammals include, but are not limited to; livestock, such as, cattle, sheep, swine, deer, and goats; sport and companion animals, such as, dogs, cats, and horses; and research animals, such as, mice, rats, rabbits, and guinea pigs. Preferably, the subject is a human.

The term “treating”, and grammatical variations thereof as used herein refers to both therapeutic and prophylactic or preventative measures, wherein the object is to prevent or slow down the targeted conditions. For example, a subject is treated for SARS CoV-2 if, after receiving a therapeutic dose of a polypeptide or polynucleotide as described herein, the subject shows an observable and/or measurable reduction in viral titer and/or relief to some extent, of one or more symptoms associated with covid-19, including reduced morbidity and mortality and/or improvement in quality of life.

The term “vaccine” and grammatical variations as used herein refers to a substance that stimulates an immune response, i.e., that induces the activation of immune cells that provide immunity against disease and/or reduce disease. Preferably the immune response is the stimulation of B and/or T cells.

The term “subunit vaccine” as used herein refers to a vaccine prepared from a portion of an antigen that immunogenic and/or antigenic, e.g., the subunit of a viral protein that is isolated and prepared as an antigen.

The term “pharmaceutically acceptable carrier or excipient” means an excipient or carrier that is compatible with the other ingredients of the composition, and not harmful to the subject to whom the composition is administered.

Examples of suitable pharmaceutically acceptable carriers and excipients are described in Remington's Pharmaceutical Sciences 18th Ed., Gennaro, ed. (Mack Publishing Co. 1990).

Numerous pharmaceutically acceptable carriers and excipients are approved by relevant government regulatory agencies. Examples of pharmaceutically acceptable carriers and excipients include sterile liquids such as water and oils, including animal, vegetable, synthetic or petroleum oils, saline solutions, aqueous dextrose and glycerol solutions, starch glucose, lactose, sucrose, gelatine, sodium stearate, glycerol monostearate, sodium chloride, propylene glycol, ethanol, wetting agents, emulsifying agents, binders, dispersants, thickeners, lubricants, pH adjusters, solubilizers, softening agents, surfactants and the like.

The pharmaceutical composition or vaccine of the invention is formulated so as to allow the active agents within to be bioavailable upon administration to a subject. For parenteral administration, the compositions and vaccines can be formulated as known in the art, for example, in a sterile aqueous solution, suspension or emulsion that optionally comprises other substances, such as salt or glucose, but not limited thereto. The pharmaceutical composition may include one or more of the following carriers or excipients: sterile diluents such as water, saline solution, Ringer's solution, isotonic sodium chloride, fixed oils such as squalene, mineral oil, mannide monooleate, cholesterol, mono or di-glycerides, polyethylene glycols, glycerine, propylene glycol, antibacterial agents such as methyl paraben or benzyl alcohol, antioxidants such as ascorbic acid or sodium bisulfite, and chelating agents such as ethylenediaminetetraacetic acid, buffers such as acetates, citrates or phosphates.

The pharmaceutical composition or vaccine of the invention may also include components such as, but not limited to, water-in-oil emulsions, liposomes, micellar components, microparticles, biodegradable microcapsules and liposomes.

The compositions and vaccines described herein can be presented in unit dosage form and can be prepared by any of the methods well known in the art of pharmacy. The term “unit dosage form” means a single dose wherein all active and inactive ingredients are combined in a suitable system, such that the patient or person administering the drug can open a single container or package with the entire dose contained therein and does not have to mix any components together from two or more containers or packages. Any examples of unit dosage forms are not intended to be limiting in any way, but merely to represent typical examples in the pharmacy arts of unit dosage forms.

Immunization, or vaccination, involves the administration of a substance (an antigen) to a patient in order to induce an immune response against said antigen. The purpose of immunization can be to prevent a disease (prophylactic immunization) or to treat an existing disease (therapeutic immunization). In the present application, antigens are derived from the S-protein of the SARS CoV-2 virus and used for a therapeutic immunization against Covid-19.

Specific immune responses against antigens can often be further stimulated in the vaccine context by the co-administration of adjuvants. Adjuvants are known in the art to accelerate, prolong, or enhance the quality of the specific immune response to antigens. An important function of an adjuvant is to overcome the poor immunogenicity typically observed in subunit vaccines by improving pathogen recognition and increasing the immune response to one that is similar to the natural innate immune response. Many different types of adjuvants have been described in the art.

In some embodiments a polypeptide or polynucleotide vaccine as described herein is formulated with an adjuvant. In one embodiment the adjuvant is an oil emulsion or oil-in-water emulsion adjuvant. In one embodiment the oil emulsion or oil-in-water adjuvant is selected from the group consisting of MF59, Addavax, AS03, and Alhydrogel. Methods of making and using oil-in-water emulsions for use as vaccine adjuvants are known in the art, as described, for example, in U.S. Pat. No. 8,778,275 (which is herein incorporated by reference in its entirety).

In one embodiment the adjuvant is an adjuvant that can be used in pre-clinical trials. In one embodiment the adjuvant is selected from the group consisting of AddaVax (a research grade equivalent of MF59, squalene-Oil-in-water), Seppivac SWE, AddaS03 (a research grade, AS03-like vaccine adjuvant) and Quil-A (a research grade, saponin vaccine adjuvant, combined with MPL-A to generate AS01-like vaccine adjuvant).

In one embodiment the adjuvant is one that is approved for human use. In one embodiment the adjuvant is selected from the group consisting of MF59, Seppivac SWE, AS03, AS01 and Alhydrogel+CpG. In one embodiment the adjuvant is selected from the group consisting of MF59, Seppivac SWE and Alhydrogel+CpG.

It is intended that reference to a range of numbers disclosed herein (for example 1 to 10) also incorporates reference to all related numbers within that range (for example, 1, 1.1, 2, 3, 3.9, 4, 5, 6, 6.5, 7, 8, 9 and 10) and also any range of rational numbers within that range (for example 2 to 8, 1.5 to 5.5 and 3.1 to 4.7) and, therefore, all sub-ranges of all ranges expressly disclosed herein are expressly disclosed. These are only examples of what is specifically intended and all possible combinations of numerical values between the lowest value and the highest value enumerated are to be considered to be expressly stated in this application in a similar manner.

### DETAILED DESCRIPTION

The present invention is based on the inventor's determination that fusion polypeptides comprising various portions of a SARS CoV-2 wild-type S-protein can be expressed in surprisingly high quantities if certain peptide domains of the S-protein are relocated to non-naturally occurring positions within the expressed fusion polypeptide.

These fusion polypeptides are immunogenic and act to increase both the humoral (B cell) and cellular (T cell) immune responses in a subject as disclosed herein. Also disclosed herein are polynucleotides encoding, and that can be transcribed and translated into, the fusion polypeptides as described herein.

In particular, the inventors have found that high levels of immunogenic fusion polypeptides can be produced by expressing a fusion polypeptide comprising a SARS CoV-2 peptide domain rearrangement in which the positions of the amino acid sequence of the N-terminal domain (NTD) and the amino acid sequence of the receptor binding domain (RBD) of the SARS CoV-2 wild-type S-protein are reversed within the polypeptide relative to what is found in nature. This does not mean that the primary amino acid sequence of these peptide domains is reversed. Rather, this means that the positions of these domains (which retain the same primary amino acid sequence as in the wild-type S-protein), is reversed, within the expressed fusion polypeptide, relative to their positions in the naturally occurring S-protein. Without wishing to be bound by theory, the inventors believe that this rearrangement provides for the dual ability of a fusion polypeptide as described herein to elicit both B-cell and T-cell immune responses in a subject, thereby providing an enhanced and long-lasting immune response.

Specifically, in a fusion polypeptide as described herein, the position of the amino acid sequence of the NTD of the SARS CoV-2 S-protein is between the C-terminus of the fusion polypeptide and the amino acid sequence of at least one RBD of the SARS CoV-2 S-protein. In some embodiments the amino acid sequence of the NTD is positioned between the C-terminus of the fusion polypeptide and the amino acid sequence of an RBD dimer.

As the skilled person will appreciate, the fusion polypeptide described herein is an artificial, non-naturally occurring antigenic or immunogenic polypeptide. The present inventors have shown that such a SARS CoV-2 fusion polypeptide produced based on the description provided herein is antigenic, retaining sufficient immunogenicity to act as a powerful vaccine against SARS CoV-2.

Accordingly, the invention disclosed herein relates generally to a SARS CoV-2 antigen that is a fusion polypeptide comprising portions of the RBD of a SARS CoV-2 wild-type S-protein and the NTD of a SARS CoV-2 wild-type S-protein, wherein the positions RBD and NTD portions are reversed within the polypeptide compared to their naturally occurring positions in the wild-type SARS CoV-2 S-protein. In some embodiments the portion of the RBD is comprised in an RBD dimer.

As used herein, the terminology “wild-type SARS CoV-2 S-protein” means the UniProt amino acid sequence having Accession No: PODTC2. The nucleic acid sequence encoding the wild-type SARS CoV-2 S-protein is the GenBank nucleic acid sequence having Accession No.: YP_009724390.1.

The amino acid sequence of the SARS CoV-2 wild-type S-protein is shown in SEQ ID NO: 19.

>sp|P0DTC2|SPIKE_SARS-COV-2 Spike glycoprotein
(SEQ ID NO: 19)
MFVFLVLLPLVSSQCVNLTTRTQLPPAYTNSFTRGVYYPDKVFRSSVLHS
TQDLFLPFFSNVTWFHAIHVSGTNGTKRFDNPVLPFNDGVYFASTEKSNI
IRGWIFGTTLDSKTQSLLIVNNATNVVIKVCEFQFCNDPFLGVYYHKNNK
SWMESEFRVYSSANNCTFEYVSQPFLMDLEGKQGNFKNLREFVFKNIDGY
FKIYSKHTPINLVRDLPQGFSALEPLVDLPIGINITRFQTLLALHRSYLT
PGDSSSGWTAGAAAYYVGYLQPRTFLLKYNENGTITDAVDCALDPLSETK
CTLKSFTVEKGIYQTSNFRVQPTESIVRFPNITNLCPFGEVFNATRFASV
YAWNRKRISNCVADYSVLYNSASFSTFKCYGVSPTKLNDLCFTNVYADSF
VIRGDEVRQIAPGQTGKIADYNYKLPDDFTGCVIAWNSNNLDSKVGGNYN
YLYRLFRKSNLKPFERDISTEIYQAGSTPCNGVEGFNCYFPLQSYGFQPT
NGVGYQPYRVVVLSFELLHAPATVCGPKKSTNLVKNKCVNFNFNGLTGTG
VLTESNKKFLPFQQFGRDIADTTDAVRDPQTLEILDITPCSFGGVSVITP
GTNTSNQVAVLYQDVNCTEVPVAIHADQLTPTWRVYSTGSNVFQTRAGCL
IGAEHVNNSYECDIPIGAGICASYQTQTNSPRRARSVASQSIIAYTMSLG
AENSVAYSNNSIAIPTNFTISVTTEILPVSMTKTSVDCTMYICGDSTECS
NLLLQYGSFCTQLNRALTGIAVEQDKNTQEVFAQVKQIYKTPPIKDFGGF
NFSQILPDPSKPSKRSFIEDLLFNKVTLADAGFIKQYGDCLGDIAARDLI
CAQKFNGLTVLPPLLTDEMIAQYTSALLAGTITSGWTFGAGAALQIPFAM
QMAYRFNGIGVTQNVLYENQKLIANQFNSAIGKIQDSLSSTASALGKLQD
VVNQNAQALNTLVKQLSSNFGAISSVLNDILSRLDKVEAEVQIDRLITGR
LQSLQTYVTQQLIRAAEIRASANLAATKMSECVLGQSKRVDFCGKGYHLM
SFPQSAPHGVVFLHVTYVPAQEKNFTTAPAICHDGKAHFPREGVFVSNGT
HWFVTQRNFYEPQIITTDNTFVSGNCDVVIGIVNNTVYDPLQPELDSFKE
ELDKYFKNHTSPDVDLGDISGINASVVNIQKEIDRLNEVAKNLNESLIDL
QELGKYEQYIKWPWYIWLGFIAGLIAIVMVTIMLCCMTSCCSCLKGCCSC
GSCCKFDEDDSEPVLKGVKLHYT

The nucleic acid sequence encoding SEQ ID NO: 19 is given herein as SEQ ID NO: 20.

Various aspects and embodiments of the invention are set out and specifically identified in the present application with reference to the numbering of the amino acid positions of SEQ ID NO: 19 as follows:
a. a S-protein leader peptide (residues 1-12) fused to
b. the amino acid sequence encoding the N-terminal domain (NTD) (residues 13-290) fused to
c. amino acid residues 291-317 fused to
d. the amino acid sequence encoding the receptor binding domain (RBD) (residues 319 to 537).

As used herein, the amino acid residues of the N-terminal domain (NTD) of the wild-type S-protein are amino acid residues 13-290 of SEQ ID NO: 19.

As used herein, the amino acid residues of the receptor binding domain (RBD) of the wild-type S-protein means amino acid residues 319 to 537 of SEQ ID NO: 19.

A fusion polypeptide as disclosed herein comprises an NTD that does not comprise an amino acid residue having a terminal amino group. Rather, the NTD of the S-protein has been moved to a new position within the fusion polypeptide relative to the RBD of the wild-type S-protein and is now located between the RBD and the carboxy-terminal end of the polypeptide.

As a further feature of the fusion polypeptides described herein, the amino acid sequences of the NTD and the at least one RBD are linked to each other by a sequence of amino acid residues that is not the naturally occurring sequence of amino acid residues found in the wild-type SARS CoV-2 S-protein.

The inventors have also determined that the expression of the fusion polypeptides described herein as antigenic and/or immunogenic polypeptide can be increased by including a peptide leader sequence in the polypeptide that drives entry of the expressed polypeptide to the endoplasmic reticulum.

The inventors believe that based on the present description, together with what is known and used in the art, a skilled worker can express an antigenic and/or immunogenic fusion polypeptide as described herein that stimulates a dual immune response comprising antibody production and T cell recruitment at an appropriate scale for commercial use, including but not limited to use as a vaccine including a subunit vaccine. This is in contrast to many other fusion polypeptides that could be (or have been) constructed to comprise various SARS CoV-2 S-protein amino acid sequences (including RBD dimers), but which either do not express, or which express poorly, and in insufficient amounts to be useful for downstream processing and/or to be considered candidates for expression optimization, i.e., that do not work.

### Polypeptides

The amino acid sequence of the expressed fusion polypeptide Fc-608 as described herein before purification.

(SEQ ID NO: 13)
MDSKGSSQKGSRLLLLLVVSNLLLCQGVVSDYKDDDDKAAALEVLFQGPR
FPNITNLCPFGEVFNATRFASVYAWNRKRISNCVADYSVLYNSASFSTFK
CYGVSPTKLNDLCFTNVYADSFVIRGDEVRQIAPGQTGKIADYNYKLPDD
FTGCVIAWNSNNLDSKVGGNYNYLYRLFRKSNLKPFERDISTEIYQAGST
PCNGVEGFNCYFPLQSYGFQPTNGVGYQPYRVVVLSFELLHAPATVCGPK
KSTNLVKNKGSGRVQPTESIVRFPNITNLCPFGEVFNATRFASVYAWNRK
RISNCVADYSVLYNSASFSTFKCYGVSPTKLNDLCFTNVYADSFVIRGDE
VRQIAPGQTGKIADYNYKLPDDFTGCVIAWNSNNLDSKVGGNYNYLYRLF
RKSNLKPFERDISTEIYQAGSTPCNGVEGFNCYFPLQSYGFQPTNGVGYQ
PYRVVVLSFELLHAPATVCGPKKSTNLVKNKSGSGSSQCVNLTTRTQLPP
AYTNSFTRGVYYPDKVFRSSVLHSTQDLFLPFFSNVTWFHAIHVSGTNGT
KRFDNPVLPFNDGVYFASTEKSNIIRGWIFGTTLDSKTQSLLIVNNATNV
VIKVCEFQFCNDPFLGVYYHKNNKSWMESEFRVYSSANNCTFEYVSQPFL
MDLEGKQGNFKNLREFVFKNIDGYFKIYSKHTPINLVRDLPQGFSALEPL
VDLPIGINITRFQTLLALHRSYLTPGDSSSGWTAGAAAYYVGYLQPRTFL
LKYNENGTITDAVDALEVLFQGPDPDPEEPKSCDKTHTCPPCPAPELLGG
PSVFLFPPKPKDTLMISRTPEVTCVVVDVSHEDPEVKFNWYVDGVEVHNA
KTKPREEQYNSTYRVVSVLTVLHQDWLNGKEYKCKVSNKALPAPIEKTIS
KAKGQPREPQVYTLPPSRDELTKNQVSLTCLVKGFYPSDIAVEWESNGQP
ENNYKTTPPVLDSDGSFFLYSKLTVDKSRWQQGNVFSCSVMHEALHNHYT
QKSLSLSPGK.

The nucleic acid sequence encoding SEQ ID NO: 13 is given herein as SEQ ID NO: 14.

Various features set out and specifically identified in the present disclosure with reference to the numbering of the amino acid positions of SEQ ID NO: 13 are shown below. These features are set out as an example of how a fusion polypeptide as described herein can be expressed as an unpurified fusion polypeptide, which is then purified by a skilled worker based on what is known and used in the art.

The skilled person will appreciate that there are many choices that can be made in designing an unpurified fusion polypeptide, which upon purification, will yield an antigenic and/or immunogenic fusion polypeptide as described herein, i.e., an S-protein fusion polypeptide that comprises an RBD dimer between the N-terminal of the fusion polypeptide, and a portion of the NTD. In particular, the skilled person recognizes that design choices can be made with regards to a, b, c, d, f, h, j, k, and l that will allow the skilled person to express a fusion polypeptide as described herein, that embodies the inventive concept of a fusion polypeptide having in the following order, an N-terminal-e-g-i.

Certain design choices with regards to a, b, c, d, f, h, j, k, and l are exemplified and disclosed herein as follows:
a. a prolactin leader peptide (residues 1-30) (SEQ ID NO: 15) fused to
b. a FLAG epitope (residues 31-38) fused to
c. residues 39-41 fused to
d. an HRV 3C protease recognition site (residues 42-49) fused to
e. a portion of the receptor binding domain (RBD1) of a SARS CoV-2 wild-type S-protein (residues 50 to 259) (SEQ ID NO: 3) fused to
f. a non-immunogenic amino acid linker exemplified as GSG (residues 260-262) fused to
g. a portion of the RBD (RBD2) of the SARS CoV-2 wild-type S-protein (residues 263-481) (SEQ ID NO: 5) fused to
h. a non-immunogenic amino acid linker exemplified here as SGSG (residues 482-484) (SEQ ID NO: 7) fused to
i. a portion of the N-terminal domain (NTD) of SARS CoV-2 wild-type S-protein (positions 485 764) (SEQ ID NO: 9) fused to
j. alanine (A) in position 765 fused to
k. an HRV 3C protease recognition site (residues 766-773) fused to
l. a human Fc sequence (residues 774-1010).

The amino acid sequences of the expressed, purified fusion polypeptide Fc-608 as described herein:

(SEQ ID NO: 1)
RFPNITNLCPFGEVFNATRFASVYAWNRKRISNCVADYSVLYNSASFSTF
KCYGVSPTKLNDLCFTNVYADSFVIRGDEVRQIAPGQTGKIADYNYKLPD
DFTGCVIAWNSNNLDSKVGGNYNYLYRLFRKSNLKPFERDISTEIYQAGS
TPCNGVEGFNCYFPLQSYGFQPTNGVGYQPYRVVVLSFELLHAPATVCGP
KKSTNLVKNKGSGRVQPTESIVRFPNITNLCPFGEVFNATRFASVYAWNR
KRISNCVADYSVLYNSASFSTFKCYGVSPTKLNDLCFTNVYADSFVIRGD
EVRQIAPGQTGKIADYNYKLPDDFTGCVIAWNSNNLDSKVGGNYNYLYRL
FRKSNLKPFERDISTEIYQAGSTPCNGVEGFNCYFPLQSYGFQPTNGVGY
QPYRVVVLSFELLHAPATVCGPKKSTNLVKNKSGSGSSQCVNLTTRTQLP
PAYTNSFTRGVYYPDKVFRSSVLHSTQDLFLPFFSNVTWFHAIHVSGTNG
TKRFDNPVLPFNDGVYFASTEKSNIIRGWIFGTTLDSKTQSLLIVNNATN
VVIKVCEFQFCNDPFLGVYYHKNNKSWMESEFRVYSSANNCTFEYVSQPF
LMDLEGKQGNFKNLREFVFKNIDGYFKIYSKHTPINLVRDLPQGFSALEP
LVDLPIGINITRFQTLLALHRSYLTPGDSSSGWTAGAAAYYVGYLQPRTF
LLKYNENGTITDAVD

The nucleic acid sequence encoding SEQ ID NO: 1 is given herein as SEQ ID NO: 2.

Various aspects and embodiments of the invention are set out and specifically identified in the present application with reference to the numbering of the amino acid positions of SEQ ID NO: 1 as follows:
a. the amino acid sequence of a first portion of the receptor binding domain (RBD1) of the SARS CoV-2 wild-type S-protein (SEQ ID NO: 3)(residues 1-210 of SEQ ID NO: 1)
b. the amino acid sequence of a first non-immunogenic linker (GSG) fused to (211-213 of SEQ ID NO: 1)
c. the amino acid sequence of a second portion of the RBD (RBD2) of the SARS CoV-2 wild-type S-protein (SEQ ID NO: 5) (residues 214-432 of SEQ ID NO: 1)
d. the amino acid sequence of a second non-immunogenic linker (SEQ ID NO: 7) (residues 433-436 of SEQ ID NO: 1)
e. the amino acid sequence encoding a portion of the N-terminal domain (NTD) of the SARS CoV-2 wild-type S-protein (SEQ ID NO: 9) (residues 437-715 of SEQ ID NO: 1).

The amino acid sequence of an RBD dimer of Fc-608 as described herein:

(SEQ ID NO: 11)
RFPNITNLCPFGEVFNATRFASVYAWNRKRISNCVADYSVLYNSASFSTF
KCYGVSPTKLNDLCFTNVYADSFVIRGDEVRQIAPGQTGKIADYNYKLPD
DFTGCVIAWNSNNLDSKVGGNYNYLYRLFRKSNLKPFERDISTEIYQAGS
TPCNGVEGFNCYFPLQSYGFQPTNGVGYQPYRVVVLSFELLHAPATVCGP
KKSTNLVKNKGSGRVQPTESIVRFPNITNLCPFGEVFNATRFASVYAWNR
KRISNCVADYSVLYNSASFSTFKCYGVSPTKLNDLCFTNVYADSFVIRGD
EVRQIAPGQTGKIADYNYKLPDDFTGCVIAWNSNNLDSKVGGNYNYLYRL
FRKSNLKPFERDISTEIYQAGSTPCNGVEGFNCYFPLQSYGFQPTNGVGY
QPYRVVVLSFELLHAPATVCGPKKSTNLVKNK.

The nucleic acid sequence encoding SEQ ID NO: 11 is given herein as SEQ ID NO: 12.

Various aspects and embodiments of the invention are set out and specifically identified in the present application with reference to the numbering of the amino acid positions of SEQ ID NO: 11 as follows:
a. the amino acid sequence of a first portion of the receptor binding domain (RBD1) of the SARS CoV-2 wild-type S-protein (SEQ ID NO: 3)(residues 1-210 of SEQ ID NO: 1)
b. the amino acid sequence of a first non-immunogenic linker (GSG) fused to (211-213 of SEQ ID NO: 1)
c. the amino acid sequence of a second portion of the RBD (RBD2) of the SARS CoV-2 wild-type S-protein (SEQ ID NO: 5) (residues 214-432 of SEQ ID NO: 1)

The amino acid sequence of the expressed fusion polypeptide Fc-628 as described herein before purification.

(SEQ ID NO: 29)
MDSKGSSQKGSRLLLLLVVSNLLLCQGVVSDYKDDDDKAAALEVLFQGPR
VQPTESIVRFPNITNLCPFGEVFNATRFASVYAWNRKRISNCVADYSVLY
NSASFSTFKCYGVSPTKLNDLCFTNVYADSFVIRGDEVRQIAPGQTGKIA
DYNYKLPDDFTGCVIAWNSNNLDSKVGGNYNYRYRLFRKSNLKPFERDIS
TEIYQAGSKPCNGVEGFNCYFPLQSYGFQPTNGVGYQPYRVVVLSFELLH
APATVCGPKKSTNLVKNKGRVQPTESIVRFPNITNLCPFGEVFNATRFAS
VYAWNRKRISNCVADYSVLYNSASFSTFKCYGVSPTKLNDLCFTNVYADS
FVIRGDEVRQIAPGQTGKIADYNYKLPDDFTGCVIAWNSNNLDSKVGGNY
NYRYRLFRKSNLKPFERDISTEIYQAGSKPCNGVEGFNCYFPLQSYGFQP
TNGVGYQPYRVVVLSFELLHAPATVCGPKKSTNLVKNKGSSQCVNLRTRT
QLPPAYTNSFTRGVYYPDKVFRSSVLHSTQDLFLPFFSNVTWFHAIHVSG
TNGTKRFDNPVLPFNDGVYFASTEKSNIIRGWIFGTTLDSKTQSLLIVNN
ATNVVIKVCEFQFCNDPFLDVYYHKNNKSWMEFGVYSSANNCTFEYVSQP
FLMDLEGKQGNFKNLREFVFKNIDGYFKIYSKHTPINLVRDLPQGFSALE
PLVDLPIGINITRFQTLLALHRSYLTPGDSSSGWTAGAAAYYVGYLQPRT
FLLKYNENGTITDAVDALEVLFQGPDPDPEEPKSCDKTHTCPPCPAPELL
GGPSVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIEKT
ISKAKGQPREPQVYTLPPSRDELTKNQVSLTCLVKGFYPSDIAVEWESNG
QPENNYKTTPPVLDSDGSFFLYSKLTVDKSRWQQGNVFSCSVMHEALHNH
YTQKSLSLSPGK.

The nucleic acid sequence encoding SEQ ID NO: 29 is given herein as SEQ ID NO: 30.

As noted previously herein, various features set out and specifically identified in the present disclosure with reference to the numbering of the amino acid positions of SEQ ID NO: 29 are shown below. These features are set out as an example of how a fusion polypeptide as described herein can be expressed as an unpurified fusion polypeptide, which is then purified by a skilled worker based on what is known and used in the art.

Also as noted previously herein, the skilled person will appreciate that there are many choices that can be made in designing an unpurified fusion polypeptide, which upon purification, will yield an antigenic and/or immunogenic fusion polypeptide as described herein, i.e., an S-protein fusion polypeptide that comprises an RBD dimer between the N-terminal of the fusion polypeptide, and a portion of the NTD. In particular, the skilled person recognizes that design choices can be made with regards to a, b, c, d, f, h, j, k, and l that will allow the skilled person to express a fusion polypeptide as described herein, that embodies the inventive concept of a fusion polypeptide having in the following order, an N-terminal-e-g-i.

Certain design choices with regards to a, b, c, d, f, h, j, k, and l are exemplified and disclosed herein as follows:
a. a prolactin leader peptide (residues 1-30) (SEQ ID NO: 15) fused to
b. a FLAG epitope (residues 31-38) fused to
c. residues 39-41 fused to
d. an HRV 3C protease recognition site (residues 42-49) fused to
e. a portion of the receptor binding domain (RBD1) of a SARS CoV-2 wild-type S-protein (residues 50 to 268) (SEQ ID NO: 23) fused to
f. a non-immunogenic amino acid linker exemplified as G in position 269 fused to
g. a portion of the RBD (RBD2) of the SARS CoV-2 wild-type S-protein (residues 270-488) (SEQ ID NO: 23) fused to
h. a non-immunogenic amino acid linker exemplified here as Sg in positions 489 and 490 fused to
i. a portion of the N-terminal domain (NTD) of SARS CoV-2 wild-type S-protein (positions 491
766) (SEQ ID NO: 25) fused to
j. alanine (A) in position 767 fused to
k. an HRV 3C protease recognition site (residues 768-775) fused to
l. a human Fc sequence (residues 774-1010).

The amino acid sequences of the expressed, purified fusion polypeptide Fc-628 as described herein:

(SEQ ID NO: 21)
RVQPTESIVRFPNITNLCPFGEVFNATRFASVYAWNRKRISNCVADYSVL
YNSASFSTFKCYGVSPTKLNDLCFTNVYADSFVIRGDEVRQIAPGQTGKI
ADYNYKLPDDFTGCVIAWNSNNLDSKVGGNYNYRYRLFRKSNLKPFERDI
STEIYQAGSKPCNGVEGFNCYFPLQSYGFQPTNGVGYQPYRVVVLSFELL
HAPATVCGPKKSTNLVKNKGRVQPTESIVRFPNITNLCPFGEVFNATRFA
SVYAWNRKRISNCVADYSVLYNSASFSTFKCYGVSPTKLNDLCFTNVYAD
SFVIRGDEVRQIAPGQTGKIADYNYKLPDDFTGCVIAWNSNNLDSKVGGN
YNYRYRLFRKSNLKPFERDISTEIYQAGSKPCNGVEGFNCYFPLQSYGFQ
PTNGVGYQPYRVVVLSFELLHAPATVCGPKKSTNLVKNKGSSQCVNLRTR
TQLPPAYTNSFTRGVYYPDKVFRSSVLHSTQDLFLPFFSNVTWFHAIHVS
GTNGTKRFDNPVLPFNDGVYFASTEKSNIIRGWIFGTTLDSKTQSLLIVN
NATNVVIKVCEFQFCNDPFLDVYYHKNNKSWMEFGVYSSANNCTFEYVSQ
PFLMDLEGKQGNFKNLREFVFKNIDGYFKIYSKHTPINLVRDLPQGFSAL
EPLVDLPIGINITRFQTLLALHRSYLTPGDSSSGWTAGAAAYYVGYLQPR
TFLLKYNENGTITDAVD

The nucleic acid sequence encoding SEQ ID NO: 21 is given herein as SEQ ID NO: 22.

Various aspects and embodiments of the invention are set out and specifically identified in the present application with reference to the numbering of the amino acid positions of SEQ ID NO: 21 as follows:
a. the amino acid sequence of a first portion of the receptor binding domain (RBD1) of the SARS CoV-2 wild-type S-protein (SEQ ID NO: 23)(residues 1-219 of SEQ ID NO: 21)
b. a first non-immunogenic linker comprising G at position 220 of SEQ ID NO: 21 fused to
c. the amino acid sequence of a second portion of the RBD (RBD2) of the SARS CoV-2 wild-type S-protein (SEQ ID NO: 23) (residues 221-439 of SEQ ID NO: 21)
d. a second non-immunogenic linker comprising G followed by S in positions 440 and 441 of SEQ ID NO: 21 fused to
e. the amino acid sequence encoding a portion of the N-terminal domain (NTD) of the SARS CoV-2 wild-type S-protein (SEQ ID NO: 25) (residues 442-717 of SEQ ID NO: 21).

The amino acid sequence of an RBD dimer of Fc-628 as described herein:

(SEQ ID NO: 27)
RVQPTESIVRFPNITNLCPFGEVFNATRFASVYAWNRKRISNCVADYSVL
YNSASFSTFKCYGVSPTKLNDLCFTNVYADSFVIRGDEVRQIAPGQTGKI
ADYNYKLPDDFTGCVIAWNSNNLDSKVGGNYNYRYRLFRKSNLKPFERDI
STEIYQAGSKPCNGVEGFNCYFPLQSYGFQPTNGVGYQPYRVVVLSFELL
HAPATVCGPKKSTNLVKNKGRVQPTESIVRFPNITNLCPFGEVFNATRFA
SVYAWNRKRISNCVADYSVLYNSASFSTFKCYGVSPTKLNDLCFTNVYAD
SFVIRGDEVRQIAPGQTGKIADYNYKLPDDFTGCVIAWNSNNLDSKVGGN
YNYRYRLFRKSNLKPFERDISTEIYQAGSKPCNGVEGFNCYFPLQSYGFQ
PTNGVGYQPYRVVVLSFELLHAPATVCGPKKSTNLVKNK.

The nucleic acid sequence encoding SEQ ID NO: 27 is given herein as SEQ ID NO: 28.

Various aspects and embodiments of the invention are set out and specifically identified in the present application with reference to the numbering of the amino acid positions of SEQ ID NO: 27 as follows:
a. the amino acid sequence of a first portion of the receptor binding domain (RBD1) of the SARS CoV-2 wild-type S-protein (SEQ ID NO: 23)(residues 1-219 of SEQ ID NO: 21)
b. a first non-immunogenic linker comprising G at position 220 of SEQ ID NO: 21 fused to
c. the amino acid sequence of a second portion of the RBD (RBD2) of the SARS CoV-2 wild-type S-protein (SEQ ID NO: 23) (residues 221-439 of SEQ ID NO: 21).

In one aspect the present invention relates to fusion polypeptide or functional fragment or variant thereof comprising at least one SARS CoV-2 Spike protein (S-protein) receptor binding domain dimer (RBD dimer) fused to at least a portion of a SARS CoV-2 N-terminal domain (NTD) by a non-immunogenic amino acid linker, wherein the RBD dimer is located between the N-terminus of the fusion polypeptide and the portion of the NTD.

In one embodiment the fusion polypeptide or functional fragment or variant thereof comprises one RBD dimer.

In one embodiment the RBD dimer comprises two copies of SEQ ID NO: 3 or a functional fragment or variant thereof. In one embodiment the RBD dimer comprises SEQ ID NO: 5 or a functional fragment or variant thereof.

In one embodiment the RBD dimer comprises two copies of SEQ ID NO: 5 or functional fragments or variants thereof.

In one embodiment RBD dimer comprises two copies of SEQ ID NO: 3 and one copy of SEQ ID NO: 5 or functional fragments or variants thereof.

In one embodiment the RBD dimer is or comprises a tandem repeat of at least a portion of SEQ ID NO: 5 or a functional fragment thereof.

In one embodiment the RBD dimer is or comprises a tandem repeat of at least a portion, preferably all of, SEQ ID NO: 3 or a functional fragment or variant thereof.

In one embodiment the RBDs in the RBD dimer are fused to each other by an amino acid linker. In one embodiment the RBDs in the RBD dimer are fused to each other in a tandem repeat.

In one embodiment the amino acid linker is a non-immunogenic linker comprising, consisting, or consisting essentially of an amino acid sequence that is non-immunogenic. In one embodiment the non-immunogenic linker is non-antigenic.

In one embodiment the non-immunogenic linker comprises 1, 2, 3, 4, 5, 6, 7, 8, 9, or 10 amino acid residues, preferably 2, 3 4, 5 or 6, preferably 2, 3, 4 or 5, preferably 3 or 4 amino acid residues.

In one embodiment the amino acid residues in the non-immunogenic linker are small neutral or non-polar amino acid residues.

In one embodiment the small neutral or non-polar amino acid residues are selected from the group consisting of S, G, and A. Preferably the amino acid residues are S or G or both.

In one embodiment the non-immunogenic linker comprises, consists, or consists essentially of GSG.

In one embodiment the RBD dimer comprises at least 95% amino acid sequence identity to SEQ ID NO: 11.

In one embodiment the RBD dimer comprises at least 96%, 97%, 98%, or 99% sequence identity to SEQ ID NO: 11.

In one embodiment the RBD dimer comprises, consists, or consists essentially of SEQ ID NO: 11 wherein the amino acid residues at positions 211-213 are selected from the group consisting of small neutral and small non-polar amino acids.

In one embodiment the small neutral or non-polar amino acid residues are selected from the group consisting of S, G, and A.

In one embodiment the amino acid residues are S or G or both.

In one embodiment the amino acid residues at positions 211-213 are GSG.

In one embodiment the RBD dimer comprises, consists, or consists essentially of SEQ ID NO: 11.

In one embodiment the portion of the NTD comprises, consists, or consists essentially of SEQ ID NO: 9.

In one embodiment the at least one RBD dimer and the portion of the NTD or functional fragments or variants thereof in the fusion polypeptide are fused to each other by an amino acid linker. In one embodiment the amino acid linker is a non-immunogenic linker comprising, consisting, or consisting essentially of an amino acid sequence that is non-immunogenic. In one embodiment the non-immunogenic linker is non-antigenic. In one embodiment the subject is an animal, preferably a mammal, preferably a human.

In one embodiment the non-immunogenic linker comprises 1, 2, 3, 4, 5, 6, 7, 8, 9, or 10 amino acid residues, preferably 2, 3 4, 5 or 6, preferably 2, 3, 4 or 5, preferably 3 or 4 amino acid residues.

In one embodiment the amino acid residues in the non-immunogenic linker are small neutral or non-polar amino acid residues.

In one embodiment the small neutral or non-polar amino acid residues are selected from the group consisting of S, G, and A. Preferably the amino acid residues are S or G or both.

In one embodiment the non-immunogenic amino acid linker comprises, consists of, or consists essentially of SEQ ID NO: 7.

In one embodiment the fusion polypeptide comprises, consists, or consists essentially of SEQ ID NO: 1, wherein the amino acid residues at positions 211-213 and 432-436 are small neutral or small non-polar amino acid residues. Preferably the small neutral or small non-polar amino acid residues are selected from the group consisting of S, G, and A.

In one embodiment the RBD dimer comprises two copies of SEQ ID NO: 23 or a functional fragment or variant thereof.

In one embodiment the RBD dimer is or comprises a tandem repeat of at least a portion of SEQ ID NO: 23 or a functional fragment or variant thereof.

In one embodiment the RBD dimer is or comprises a tandem repeat of at least a portion, preferably all of, SEQ ID NO: 23 or a functional fragment or variant thereof.

In one embodiment the RBD dimer is or comprises a tandem repeat of at least a portion, preferably all of, SEQ ID NO: 23 or a functional fragment or variant thereof wherein the copies of the RBD in the tandem repeat are fused by a non-immunogenic amino acid linker.

In one embodiment the non-immunogenic linker comprises 1, 2, 3, 4, 5, 6, 7, 8, 9, or 10 amino acid residues, preferably 2, 3 4, 5 or 6, preferably 2, 3, 4 or 5, preferably 1 or 2 amino acid residues.

In one embodiment the non-immunogenic linker comprises or consists of a G linking two copies of SEQ ID NO: 23 in the tandem repeat.

In one embodiment the RBD dimer comprises at least 96%, 97%, 98%, or 99% sequence identity to SEQ ID NO: 27.

In one embodiment the RBD dimer comprises, consists, or consists essentially of SEQ ID NO: 27 wherein the amino acid residue at position 220 is selected from the group consisting of small neutral and small non-polar amino acids.

In one embodiment the small neutral or non-polar amino acid residues are selected from the group consisting of S, G, and A.

In one embodiment the amino acid residue at position 220 is G.

In one embodiment the RBD dimer comprises, consists, or consists essentially of SEQ ID NO: 27.

In one embodiment the portion of the NTD comprises, consists, or consists essentially of SEQ ID NO: 25.

In one embodiment the fusion polypeptide comprises, consists, or consists essentially of SEQ ID NO: 21, wherein the amino acid residues at positions 220 and 440-441 are small neutral or small non-polar amino acid residues. Preferably the small neutral or small non-polar amino acid residues are selected from the group consisting of S, G, and A.

In another aspect the invention relates to a fusion polypeptide or functional fragment or variant thereof comprising in the following order from the N-terminus to the C-terminus, peptide domains a), b) and c), wherein
a) is a portion of the receptor binding domain (RBD) of the SARS CoV-2 S-protein,
b) is a portion of the receptor binding domain (RBD) of the SARS CoV-2 S-protein, and
c) is a portion of the N-terminal domain (NTD) of the SARS CoV-2 S-protein.

In one embodiment the fusion polypeptide comprises d) a non-immunogenic amino acid linker fusing a) to b).

In one embodiment the fusion polypeptide comprises e) a non-immunogenic amino acid linker fusing b) to c).

In one embodiment a) is comprised by SEQ ID NO: 5. In one embodiment a) comprises at least 95% sequence identity to SEQ ID NO: 5.

In one embodiment a) comprises at least 95% sequence identity to SEQ ID NO: 3. In one embodiment a) comprises at least 96%, 97%, 98%, or 99% sequence identity to SEQ ID NO: 3.

In one embodiment a) comprises SEQ ID NO: 3. In one embodiment a) consists essentially of SEQ ID NO: 3. In one embodiment a) consists of SEQ ID NO: 3.

In one embodiment b) comprises SEQ ID NO: 5.

In one embodiment b) comprises at least 95% sequence identity to SEQ ID NO: 5. In one embodiment b) comprises at least 96%, 97%, 98%, or 99% sequence identity to SEQ ID NO: 5.

In one embodiment b) comprises SEQ ID NO: 5. In one embodiment b) consists essentially of SEQ ID NO: 5. In one embodiment b) consists of SEQ ID NO: 5.

In one embodiment b) comprises SEQ ID NO: 3.

In one embodiment b) comprises at least 95% sequence identity to SEQ ID NO: 3. In one embodiment b) comprises at least 96%, 97%, 98%, or 99% sequence identity to SEQ ID NO: 3.

In one embodiment b) comprises SEQ ID NO: 3. In one embodiment b) consists essentially of SEQ ID NO: 3. In one embodiment b) consists of SEQ ID NO: 3.

In one embodiment c) comprises SEQ ID NO: 9.

In one embodiment c) comprises at least 95% sequence identity to SEQ ID NO: 9. In one embodiment c) comprises at least 96%, 97%, 98%, or 99% sequence identity to SEQ ID NO: 9.

In one embodiment c) comprises SEQ ID NO: 9. In one embodiment c) consists essentially of SEQ ID NO: 9. In one embodiment c) consists of SEQ ID NO: 9.

In one embodiment d) or e) or both comprises 1, 2, 3, 4, 5, 6, 7, 8, 9, or 10 amino acid residues, preferably 2, 3 4, 5 or 6, preferably 2, 3, 4 or 5, preferably 3 or 4 amino acid residues.

In one embodiment the amino acid residues in d) or e) are small neutral or non-polar amino acid residues.

In one embodiment the small neutral or non-polar amino acid residues are selected from the group consisting of S, G, and A. Preferably the amino acid residues are S or G or both.

In one embodiment d) comprises GSG. In one embodiment d) consists essentially of GSG. In one embodiment d) consists of GSG.

In one embodiment e) comprises SEQ ID NO: 7. In one embodiment e) consists essentially of SEQ ID NO: 7. In one embodiment e) consists of SEQ ID NO: 7.

In one embodiment a) comprises, consists, or consists essentially of amino acid residues 1 to 210 of SEQ ID NO: 1.

In one embodiment b) comprises, consists, or consists essentially of amino acid residues 214 to 432 of SEQ ID NO: 5.

In one embodiment c) comprises, consists, or consists essentially of amino acid residues 437 to 715 of SEQ ID NO: 9.

In one embodiment d) comprises, consists, or consists essentially of amino acid residues 211 to 213 of GSG.

In one embodiment e) comprises, consists, or consists essentially of amino acid residues 433 to 436 of SEQ ID NO: 7.

In another aspect the invention relates to a fusion polypeptide or functional fragment or variant thereof comprising:
a. a first amino acid sequence comprising a first portion of the receptor binding domain (RBD) of a wild-type SARS CoV-2 S-protein,
b. a second amino acid sequence comprising a second portion of the receptor binding domain (RBD) of a wild-type SARS CoV-2 S-protein,
c. an amino acid sequence comprising at least a portion of the N-terminal domain (NTD) of a wild-type SARS CoV-2 protein,
d. a first non-immunogenic amino acid linker fusing a) to b), and
e. a second non-immunogenic amino acid linker fusing b) to c),
   wherein a) and b) are located between the N-terminus of the fusion polypeptide and c).

In one embodiment a) is comprised by SEQ ID NO: 5. In one embodiment a) comprises at least 95% sequence identity to SEQ ID NO: 5.

In one embodiment a) comprises at least 95% sequence identity to SEQ ID NO: 3. In one embodiment a) comprises at least 96%, 97%, 98%, or 99% sequence identity to SEQ ID NO: 3.

In one embodiment a) comprises SEQ ID NO: 3. In one embodiment a) consists essentially of SEQ ID NO: 3. In one embodiment a) consists of SEQ ID NO: 3.

In one embodiment b) comprises SEQ ID NO: 5.

In one embodiment b) comprises at least 95% sequence identity to SEQ ID NO: 5. In one embodiment b) comprises at least 96%, 97%, 98%, or 99% sequence identity to SEQ ID NO: 5.

In one embodiment b) comprises SEQ ID NO: 5. In one embodiment b) consists essentially of SEQ ID NO: 5. In one embodiment b) consists of SEQ ID NO: 5.

In one embodiment b) comprises SEQ ID NO: 3.

In one embodiment b) comprises at least 95% sequence identity to SEQ ID NO: 3. In one embodiment b) comprises at least 96%, 97%, 98%, or 99% sequence identity to SEQ ID NO: 3.

In one embodiment b) comprises SEQ ID NO: 3. In one embodiment b) consists essentially of SEQ ID NO: 3. In one embodiment b) consists of SEQ ID NO: 3.

In one embodiment c) comprises SEQ ID NO: 9.

In one embodiment c) comprises at least 95% sequence identity to SEQ ID NO: 9. In one embodiment c) comprises at least 96%, 97%, 98%, or 99% sequence identity to SEQ ID NO: 9.

In one embodiment c) comprises SEQ ID NO: 9. In one embodiment c) consists essentially of SEQ ID NO: 9. In one embodiment c) consists of SEQ ID NO: 9.

In one embodiment the non-immunogenic linker comprises 1, 2, 3, 4, 5, 6, 7, 8, 9, or 10 amino acid residues, preferably 2, 3 4, 5 or 6, preferably 2, 3, 4 or 5, preferably 3 or 4 amino acid residues.

In one embodiment the amino acid residues in the non-immunogenic linker are small neutral or non-polar amino acid residues.

In one embodiment the small neutral or non-polar amino acid residues are selected from the group consisting of S, G, and A. Preferably the amino acid residues are S or G or both.

In one embodiment the non-immunogenic linker comprises, consists, or consists essentially of GSG.

In one embodiment the non-immunogenic linker comprises, consists, or consists essentially of SEQ ID NO: 7.

In one embodiment a) comprises, consists, or consists essentially of amino acid residues 1 to 210 of SEQ ID NO: 1.

In one embodiment b) comprises, consists, or consists essentially of amino acid residues 214 to 432 of SEQ ID NO: 5.

In one embodiment c) comprises, consists, or consists essentially of amino acid residues 437 to 715 of SEQ ID NO: 9.

In one embodiment d) comprises, consists, or consists essentially of amino acid residues 211 to 213 of GSG.

In one embodiment e) comprises, consists, or consists essentially of amino acid residues 433 to 436 of SEQ ID NO: 7.

In another aspect the invention relates to a fusion polypeptide or functional fragment or variant thereof that comprises the following amino acid sequences in order from the N-terminal of the polypeptide: discrete amino acid sequences having at least 95% sequence identity to SEQ ID NO: 3, SEQ ID NO: 3, and SEQ ID NO 11.

In one embodiment the fusion polypeptide consists essentially of the following amino acid sequences in order from the N-terminal of the polypeptide: amino acid sequences having at least 95% sequence identity to SEQ ID NO: 3, SEQ ID NO: 3, and SEQ ID NO 11.

In one embodiment, the fusion polypeptide or a functional fragment or variant thereof comprises amino acid sequences comprising at least 96%, 97%, 98%, or 99% sequence identity to: SEQ ID NO: 3, SEQ ID NO: 3, SEQ ID NO: 9.

In one embodiment the fusion polypeptide consists essentially of amino acid sequences comprising at least 96%, 97%, 98%, or 99% sequence identity to: SEQ ID NO: 3, SEQ ID NO: 3, SEQ ID NO: 9.

In one embodiment the fusion polypeptide or a functional fragment or variant thereof comprises amino acid sequences SEQ ID NO: 3, SEQ ID NO: 3, SEQ ID NO: 9.

In one embodiment the fusion polypeptide or a functional fragment or variant thereof consists essentially of amino acid sequences SEQ ID NO: 3, SEQ ID NO: 3, SEQ ID NO: 9.

In one embodiment, the fusion polypeptide or a functional fragment or variant thereof comprises amino acid sequences comprising at least 96%, 97%, 98%, or 99% sequence identity to: SEQ ID NO: 5, SEQ ID NO: 3, SEQ ID NO: 9.

In one embodiment, the fusion polypeptide or a functional fragment or variant thereof consists essentially of amino acid sequences that have at least 96%, 97%, 98%, or 99% sequence identity to: SEQ ID NO: 5, SEQ ID NO: 3, SEQ ID NO: 9.

In one embodiment the fusion polypeptide or a functional fragment or variant thereof comprises amino acid sequences SEQ ID NO: 5, SEQ ID NO: 3, SEQ ID NO: 9.

In one embodiment the fusion polypeptide or a functional fragment or variant thereof consists essentially of amino acid sequences SEQ ID NO: 5, SEQ ID NO: 3, SEQ ID NO: 9.

In one embodiment, the fusion polypeptide or a functional fragment or variant thereof comprises amino acid sequences comprising at least 96%, 97%, 98%, or 99% sequence identity to: SEQ ID NO: 3, SEQ ID NO: 5, SEQ ID NO: 9.

In one embodiment, the fusion polypeptide or a functional fragment or variant thereof consists essentially of amino acid sequences that have at least 96%, 97%, 98%, or 99% sequence identity to: SEQ ID NO: 3, SEQ ID NO: 5, SEQ ID NO: 9.

In one embodiment the fusion polypeptide or a functional fragment or variant thereof comprises amino acid sequences SEQ ID NO: 3, SEQ ID NO: 5, SEQ ID NO: 9.

In one embodiment the fusion polypeptide or a functional fragment or variant thereof consists essentially of amino acid sequences SEQ ID NO: 3, SEQ ID NO: 5, SEQ ID NO: 9.

In one embodiment, the fusion polypeptide or a functional fragment or variant thereof comprises amino acid sequences that have at least 96%, 97%, 98%, or 99% sequence identity to: SEQ ID NO: 5, SEQ ID NO: 5, SEQ ID NO: 9.

In one embodiment, the fusion polypeptide or a functional fragment or variant thereof consists essentially of amino acid sequences that have at least 96%, 97%, 98%, or 99% sequence identity to: SEQ ID NO: 5, SEQ ID NO: 5, SEQ ID NO: 9.

In one embodiment the fusion polypeptide or a functional fragment or variant thereof comprises amino acid sequences SEQ ID NO: 5, SEQ ID NO: 5, SEQ ID NO: 9.

In one embodiment the fusion polypeptide or a functional fragment or variant thereof consists essentially of amino acid sequences SEQ ID NO: 5, SEQ ID NO: 5, SEQ ID NO: 9.

In one embodiment the fusion polypeptide or a functional fragment or variant thereof comprises at least one amino acid linker (LK) as follows:
SEQ ID NO: 3-LK—SEQ ID NO: 3-LK—SEQ ID NO: 9;
SEQ ID NO: 3-LK—SEQ ID NO: 5-LK—SEQ ID NO: 9;
SEQ ID NO: 5-LK—SEQ ID NO: 3-LK—SEQ ID NO: 9;
SEQ ID NO: 5-LK—SEQ ID NO: 5-LK—SEQ ID NO: 9.

In one embodiment the amino acid linker comprising 1, 2, 3, 4, 5, 6, 7, 8, 9, or 10 amino acid residues, preferably 2, 3 4, 5 or 6, preferably 2, 3, 4 or 5, preferably 3 or 4 amino acid residues.

In one embodiment the amino acid linker is a non-immunogenic linker.

In one embodiment the amino acid residues in the non-immunogenic linker are small neutral or non-polar amino acid residues.

In one embodiment the small neutral or non-polar amino acid residues are selected from the group consisting of S, G, A Preferably the amino acid residues are S or G or both.

In one embodiment the non-immunogenic linker comprises, consists, or consists essentially of GSG.

In one embodiment the non-immunogenic linker comprises, consists, or consists essentially of SEQ ID NO: 7.

In another aspect the invention relates to a fusion polypeptide consisting of a polypeptide or functional fragment or variant thereof comprising at least two copies of an amino acid sequence comprising 95% sequence identity to SEQ ID NO: 3 and at least one copy of an amino acid sequence comprising 95% sequence identity to SEQ ID NO: 9.

In one embodiment the polypeptide or functional fragment or variant thereof consists essentially of at least two copies of an amino acid sequence comprising 95% sequence identity to SEQ ID NO: 3 and at least one copy of an amino acid sequence comprising at least 95% sequence identity to SEQ ID NO: 9.

In one embodiment the polypeptide or functional fragment or variant thereof comprises of at least two copies of an amino acid sequence comprising at least 96%, 97%, 98%, or 99% sequence identity to SEQ ID NO: 3 and at least one copy of an amino acid sequence comprising at least 96%, 97%, 98%, or 99% sequence identity to SEQ ID NO: 9.

In one embodiment the polypeptide or functional fragment or variant thereof consists essentially of at least two copies of an amino acid sequence comprising at least 96%, 97%, 98%, or 99% sequence identity to SEQ ID NO: 3 and at least one copy of an amino acid sequence comprising at least 96%, 97%, 98%, or 99% sequence identity to SEQ ID NO: 9.

In one embodiment the polypeptide or functional fragment or variant thereof comprises at least two copies SEQ ID NO: 3 and at least one copy SEQ ID NO: 9.

In one embodiment the polypeptide or functional fragment or variant thereof consists essentially of at least two copies of SEQ ID NO: 3 and at least one copy of SEQ ID NO: 9.

In one embodiment the at least two copies of SEQ ID NO: 3 form an RBD dimer. In one embodiment the RBD dimer is positioned between the N-terminal of the polypeptide and SEQ ID NO: 9.

In one embodiment the RBD dimer comprises an amino acid linker between the two copies of SEQ ID NO: 3. Specifically contemplated embodiments of the amino acid linker in this aspect of the invention are as set out in any of the other aspects and embodiments of the invention set forth herein including the nature, length, positioning and composition of the amino acid linker, but not limited thereto.

In one embodiment the polypeptide comprises at least one copy of an amino acid sequence comprising at least 95%, 96%, 97%, 98%, or 99% sequence identity SEQ ID NO: 5.

In one embodiment the polypeptide comprises at least one copy of an amino acid sequence consisting essentially of at least 95%, 96%, 97%, 98%, or 99% sequence identity SEQ ID NO: 5.

In one embodiment the polypeptide comprises at least one copy of an amino acid sequence comprising SEQ ID NO: 5.

In one embodiment the polypeptide comprises at least one copy of an amino acid sequence that consists essentially of SEQ ID NO: 5.

In one embodiment the polypeptide comprises at least two copies of an amino acid sequence comprising at least 95%, 96%, 97%, 98%, or 99% sequence identity SEQ ID NO: 5.

In one embodiment the polypeptide comprises at least two copies of an amino acid sequence consisting essentially of at least 95, 96%, 97%, 98%, or 99% sequence identity SEQ ID NO: 5.

In one embodiment the polypeptide comprises at least two copies of an amino acid sequence comprising SEQ ID NO: 5.

In one embodiment the polypeptide comprises at least two copies of an amino acid sequence that consists essentially of SEQ ID NO: 5.

In one embodiment the at least two copies of SEQ ID NO: 5 form an RBD dimer. In one embodiment the RBD dimer is positioned between the N-terminal of the polypeptide and SEQ ID NO: 9.

In one embodiment the RBD dimer comprises an amino acid linker between the two copies of SEQ ID NO: 5. Specifically contemplated embodiments of the amino acid linker in this aspect of the invention are as set out in any of the other aspects and embodiments of the invention set forth herein including the nature, length, positioning and composition of the amino acid linker, but not limited thereto.

Without wishing to be bound by theory the inventors believe either SEQ ID NO: 3 or SEQ ID NO: 5 (which wholly encompasses SEQ ID NO: 3) or a functional fragment or variant can be used to form an RBD dimer within a fusion polypeptide as described herein.

Accordingly, contemplated as embodiments within this aspect of the invention are fusion polypeptides comprising at least one copy of SEQ ID NO: 3, one copy of SEQ ID NO: 5 and one copy of SEQ ID NO: 9, including functional fragments and variants as set forth above.

In some of these embodiments the at least one copy of SEQ ID NO: 3 and SEQ ID NO: 5 form an RBD dimer comprising or consisting essentially of SEQ ID NO: 3-LK-SEQ ID NO: 5 or SEQ ID NO: 5-LK-SEQ ID NO: 3 wherein LK is an amino acid linker as described herein.

In another aspect the invention relates to a fusion polypeptide or functional fragment or variant thereof comprising at least 95% sequence identity to SEQ ID NO: 1.

In one embodiment the fusion polypeptide or functional fragment or variant thereof consists essentially of an amino acid sequence comprising at least 95% sequence identity to SEQ ID NO: 1.

In one embodiment, the fusion polypeptide or a functional fragment or variant thereof comprises at least 96%, 97%, 98%, or 99% sequence identity to SEQ ID NO: 1. In one embodiment the polypeptide comprises SEQ ID NO: 1.

In one embodiment the fusion polypeptide or functional fragment or variant thereof consists essentially of an amino acid sequence comprising at least 96%, 97%, 98%, or 99% sequence identity to SEQ ID NO: 1. In one embodiment the polypeptide consists essentially of SEQ ID NO: 1.

In one embodiment the fusion polypeptide is isolated.

In one embodiment any variation in the amino acid residues of the fusion polypeptide or a functional fragment or variant thereof as compared to SEQ ID NO: 1 is variation in amino acid positions 211-213 and 433-436 only.

In one embodiment the variation in amino acid positions 211-213 and 433-436 comprises variation in an amino acid linker sequence. In one embodiment the amino acid linker sequence is a non-immunogenic linker. Specifically contemplated as embodiments of this aspect of the invention are all of the embodiments related to amino acid linkers and non-immunogenic linkers as set forth herein in relation to any other aspect of the invention.

In another aspect the invention relates to a fusion polypeptide or functional fragment or variant thereof comprising at least 95% sequence identity to SEQ ID NO: 21.

In one embodiment the fusion polypeptide or functional fragment or variant thereof consists essentially of an amino acid sequence comprising at least 95% sequence identity to SEQ ID NO: 21.

In one embodiment, the fusion polypeptide or a functional fragment or variant thereof comprises at least 96%, 97%, 98%, or 99% sequence identity to SEQ ID NO: 21. In one embodiment the polypeptide comprises SEQ ID NO: 21.

In one embodiment the fusion polypeptide or functional fragment or variant thereof consists essentially of an amino acid sequence comprising at least 96%, 97%, 98%, or 99% sequence identity to SEQ ID NO: 21. In one embodiment the polypeptide consists essentially of SEQ ID NO: 21.

In one embodiment the fusion polypeptide is isolated.

In one embodiment any variation in the amino acid residues of the fusion polypeptide or a functional fragment or variant thereof as compared to SEQ ID NO: 21 is variation at amino acid positions 220 and 440-441 only.

In one embodiment the variation in amino acid positions 220 and 440-441 comprises variation in an amino acid linker sequence. In one embodiment the amino acid linker sequence is a non-immunogenic linker. Specifically contemplated as embodiments of this aspect of the invention are all of the embodiments related to amino acid linkers and non-immunogenic linkers as set forth herein in relation to any other aspect of the invention.

In another aspect the invention relates to a fusion polypeptide or a functional fragment or variant thereof comprising the amino acid sequences of amino acid positions 1-210, 214-432 and 437 to 715 of SEQ ID NO: 1 or a functional fragment or variant thereof.

In one embodiment the fusion polypeptide is isolated.

In one embodiment any variation in the amino acid residues of the fusion polypeptide or a functional fragment or variant thereof as compared to SEQ ID NO: 1 is variation in amino acid positions 211-213 and 433-436 only.

In one embodiment the variation in amino acid positions 211-213 and 433-436 comprises variation in an amino acid linker sequence. In one embodiment the amino acid linker sequence is a non-immunogenic linker. Specifically contemplated as embodiments of this aspect of the invention are all of the embodiments related to amino acid linkers and non-immunogenic linkers as set forth herein in relation to any other aspect of the invention.

Specifically contemplated as embodiments of this aspect of the invention are all of the embodiments set forth in the previous fusion polypeptide aspects of the invention, particularly but not limited to the nature (including composition and length) and % sequence identity of the amino acid sequences and subsequences identified by SEQ ID NO:, the nature and the amino acid residues, including where identified by SEQ ID NO:, and the positioning of these sequences within a fusion polypeptide as described herein.

In another aspect the invention relates to a fusion polypeptide or a functional fragment or variant thereof comprising the amino acid sequences of amino acid positions 1-219, 221-439 and 442 to 717 of SEQ ID NO: 21 or a functional fragment or variant thereof.

In one embodiment the fusion polypeptide is isolated.

In one embodiment any variation in the amino acid residues of the fusion polypeptide or a functional fragment or variant thereof as compared to SEQ ID NO: 21 is variation in amino acid positions 220 and 440-441 only.

In one embodiment the variation in amino acid positions 220 and 440-441 comprises variation in an amino acid linker sequence. In one embodiment the amino acid linker sequence is a non-immunogenic linker. Specifically contemplated as embodiments of this aspect of the invention are all of the embodiments related to amino acid linkers and non-immunogenic linkers as set forth herein in relation to any other aspect of the invention.

Specifically contemplated as embodiments of this aspect of the invention are all of the embodiments set forth in the previous fusion polypeptide aspects of the invention, particularly but not limited to the nature (including composition and length) and % sequence identity of the amino acid sequences and subsequences identified by SEQ ID NO:, the nature and the amino acid residues, including where identified by SEQ ID NO:, and the positioning of these sequences within either a polypeptide as described herein.

Also specifically contemplated as an embodiment of each of the fusion polypeptide aspects described herein, the fusion polypeptide is a vaccine. In one embodiment the vaccine is a subunit vaccine. In one embodiment the fusion polypeptide is expressed from a polynucleotide sequence that can also be expressed to form an mRNA vaccine.

### Polynucleotides

In one aspect the present invention relates to a polynucleotide encoding a fusion polypeptide or functional fragment or variant thereof as described herein.

In another aspect the invention relates to a polynucleotide comprising at least 70% sequence identity to a polynucleotide encoding a fusion polypeptide or functional fragment or variant thereof comprising at least one RBD dimer fused to at least a portion of an NTD by a non-immunogenic amino acid linker, wherein the RBD dimer is located between the N-terminus of the fusion polypeptide and the portion of the NTD.

In one embodiment the polynucleotide comprises a nucleic acid sequence comprising at least 75%, 80%, 85%, 90%, 95%, 96%, 97%, 98% or 99% sequence identity to the polynucleotide encoding the fusion polypeptide or functional fragment or variant thereof.

In one embodiment the polynucleotide comprises SEQ ID NO: 12.

In one embodiment the polynucleotide encoding the RBD dimer comprises, consists, or consists essentially of a tandem repeat of SEQ ID NO: 4, at tandem repeat of SEQ ID NO: 6, or a tandem repeat of a nucleic acid sequence encoding at least a portion of an RBD comprising discrete sequence regions comprising, consisting of, or consisting essentially of SEQ ID NO: 4 and SEQ ID NO: 6.

In one embodiment the polynucleotide encoding the RBD dimer comprises, consists, or consists essentially of at least two copies of SEQ ID NO: 4, at least two copies of SEQ ID NO: 6, or at least one discrete copy of SEQ ID NO: 4 and one discrete copy of SEQ ID NO: 6. In one embodiment, the nucleic acid sequences encoding the RBD dimer are linked by a nucleic acid linker. In one embodiment the nucleic acid linker encodes an amino acid linker. In one embodiment the encoded amino acid linker is non-immunogenic linker comprising, consisting, or consisting essentially of an amino acid sequence that is non-immunogenic. In one embodiment the non-immunogenic linker is non-antigenic.

In one embodiment the nucleic acid linker encodes a non-immunogenic linker comprising 1, 2, 3, 4, 5, 6, 7, 8, 9, or 10 amino acid residues, preferably 2, 3 4, 5 or 6, preferably 2, 3, 4 or 5, preferably 3 or 4 amino acid residues.

In one embodiment the amino acid residues in the non-immunogenic linker are small neutral or non-polar amino acid residues.

In one embodiment the small neutral or non-polar amino acid residues are selected from the group consisting of S, G, and A. Preferably the amino acid residues are S or G or both.

In one embodiment the non-immunogenic linker comprised in the RBD dimer comprises, consists, or consists essentially of GGCAGCGGC.

In one embodiment the polynucleotide encoding the portion of the NTD comprises, consists, or consists essentially of SEQ ID NO: 10.

In one embodiment the polynucleotides encoding the at least one RBD dimer and the portion of the NTD are linked to each other by a nucleic acid linker. In one embodiment the nucleic acid linker encodes an amino acid linker. In one embodiment the encoded amino acid linker is non-immunogenic linker comprising, consisting, or consisting essentially of an amino acid sequence that is non-immunogenic. In one embodiment the non-immunogenic linker is non-antigenic.

In one embodiment the nucleic acid linker encodes a non-immunogenic linker comprising 1, 2, 3, 4, 5, 6, 7, 8, 9, or 10 amino acid residues, preferably 2, 3 4, 5 or 6, preferably 2, 3, 4 or 5, preferably 3 or 4 amino acid residues.

In one embodiment the amino acid residues in the non-immunogenic linker are small neutral or non-polar amino acid residues.

In one embodiment the small neutral or non-polar amino acid residues are selected from the group consisting of S, G, and A. Preferably the amino acid residues are S or G or both.

In one embodiment the nucleic acid linker comprises, consists, or consists essentially of SEQ ID NO: 8.

In one embodiment the polynucleotide comprises, consists, or consists essentially of SEQ ID NO: 2, wherein the nucleic acid sequence encoding the amino acid residues at positions 211-213 and 432-436 encodes small neutral or small non-polar amino acid residues. Preferably the small neutral or small non-polar amino acid residues are selected from the group consisting of S, G, and A.

In one embodiment the polynucleotide comprises SEQ ID NO: 22.

In one embodiment the polynucleotide encoding the RBD dimer comprises, consists, or consists essentially of a tandem repeat of SEQ ID NO: 24, or a tandem repeat of a nucleic acid sequence encoding at least a portion of an RBD comprising discrete sequence regions comprising, consisting of, or consisting essentially of SEQ ID NO: 24.

In one embodiment the polynucleotide encoding the RBD dimer comprises, consists, or consists essentially of at least two copies of SEQ ID NO: 24.

In one embodiment, the nucleic acid sequences encoding the RBD dimer are linked by a nucleic acid linker. In one embodiment the nucleic acid linker encodes an amino acid linker. In one embodiment the encoded amino acid linker is non-immunogenic linker comprising, consisting, or consisting essentially of an amino acid sequence that is non-immunogenic. In one embodiment the non-immunogenic linker is non-antigenic.

In one embodiment the nucleic acid linker encodes a non-immunogenic linker comprising 1, 2, 3, 4, 5, 6, 7, 8, 9, or 10 amino acid residues, preferably 2, 3 4, 5 or 6, preferably 2, 3, 4 or 5, preferably 1 or 2 amino acid residues.

In one embodiment the amino acid residues in the non-immunogenic linker are small neutral or non-polar amino acid residues.

In one embodiment the small neutral or non-polar amino acid residues are selected from the group consisting of S, G, and A. Preferably the amino acid residues are S or G or both. Preferably the amino acid residue is G.

In one embodiment the non-immunogenic linker comprised in the RBD dimer comprises, consists, or consists essentially of G at position 220 of SEQ ID NO: 21.

In one embodiment the polynucleotide encoding the portion of the NTD comprises, consists, or consists essentially of SEQ ID NO: 26.

In one embodiment the polynucleotides encoding the at least one RBD dimer and the portion of the NTD are linked to each other by a nucleic acid linker. In one embodiment the nucleic acid linker encodes an amino acid linker. In one embodiment the encoded amino acid linker is non-immunogenic linker comprising, consisting, or consisting essentially of an amino acid sequence that is non-immunogenic. In one embodiment the non-immunogenic linker is non-antigenic.

In one embodiment the nucleic acid linker encodes a non-immunogenic linker comprising 1, 2, 3, 4, 5, 6, 7, 8, 9, or 10 amino acid residues, preferably 2, 3 4, 5 or 6, preferably 2, 3, 4 or 5, preferably 1 or 2 amino acid residues.

In one embodiment the amino acid residues in the non-immunogenic linker are small neutral or non-polar amino acid residues.

In one embodiment the small neutral or non-polar amino acid residues are selected from the group consisting of S, G, and A. Preferably the amino acid residues are S or G or both.

In one embodiment the nucleic acid linker comprises, consists, or consists essentially of GS in positions 440 and 441 of SEQ ID NO: 21.

In one embodiment the polynucleotide comprises, consists, or consists essentially of SEQ ID NO: 22, wherein the nucleic acid sequence encoding the amino acid residues at positions 220 and 440-441 encodes small neutral or small non-polar amino acid residues. Preferably the small neutral or small non-polar amino acid residues are selected from the group consisting of S, G, and A. Preferably the amino acid residues are G and/or S.

In another aspect the invention relates to a polynucleotide comprising in the following order from 5′ to 3′, nucleic acid sequences a), b) and c), wherein
a) encodes a portion of the receptor binding domain (RBD) of the SARS CoV-2 S-protein,
b) encodes a portion of the receptor binding domain (RBD) of the SARS CoV-2 S-protein, and
c) encodes a portion of the N-terminal domain (NTD) of the SARS CoV-2 S-protein.

In one embodiment the polynucleotide comprises d) a nucleic acid linker encoding a non-immunogenic amino acid sequence linking a) to b).

In one embodiment the polynucleotide comprises e) a nucleic acid linker encoding a non-immunogenic amino acid sequence linking b) to c).

In one embodiment a) is comprised by SEQ ID NO: 6. In one embodiment a) comprises at least 95% sequence identity to SEQ ID NO: 6.

In one embodiment a) comprises at least 75%, 80%, 85%, 90%, 95%, 96%, 97%, 98%, or 99% sequence identity to SEQ ID NO: 4.

In one embodiment a) comprises SEQ ID NO: 4. In one embodiment a) consists essentially of SEQ ID NO: 4. In one embodiment a) consists of SEQ ID NO: 4.

In one embodiment b) comprises SEQ ID NO: 6.

In one embodiment b) comprises at least at least 75%, 80%, 85%, 90%, 95%, 96%, 97%, 98%, or 99% sequence identity to SEQ ID NO: 6.

In one embodiment b) comprises SEQ ID NO: 6. In one embodiment b) consists essentially of SEQ ID NO: 6. In one embodiment b) consists of SEQ ID NO: 6.

In one embodiment b) comprises SEQ ID NO: 4.

In one embodiment b) comprises at least 75%, 80%, 85%, 90%, 95%, 96%, 97%, 98%, or 99% sequence identity to SEQ ID NO: 4.

In one embodiment b) comprises SEQ ID NO: 4. In one embodiment b) consists essentially of SEQ ID NO: 4. In one embodiment b) consists of SEQ ID NO: 4.

In one embodiment c) comprises SEQ ID NO: 10.

In one embodiment c) comprises at least 75%, 80%, 85%, 90%, 95%, 96%, 97%, 98%, or 99% sequence identity to SEQ ID NO: 10.

In one embodiment c) comprises SEQ ID NO: 10. In one embodiment c) consists essentially of SEQ ID NO: 10. In one embodiment c) consists of SEQ ID NO: 10.

In one embodiment a) is comprised by SEQ ID NO: 34. In one embodiment a) comprises at least 95% sequence identity to SEQ ID NO: 34.

In one embodiment a) comprises at least 75%, 80%, 85%, 90%, 95%, 96%, 97%, 98%, or 99% sequence identity to SEQ ID NO: 33.

In one embodiment a) comprises SEQ ID NO: 33. In one embodiment a) consists essentially of SEQ ID NO: 4. In one embodiment a) consists of SEQ ID NO: 33.

In one embodiment b) comprises SEQ ID NO: 34.

In one embodiment b) comprises at least at least 75%, 80%, 85%, 90%, 95%, 96%, 97%, 98%, or 99% sequence identity to SEQ ID NO: 34.

In one embodiment b) comprises SEQ ID NO: 34. In one embodiment b) consists essentially of SEQ ID NO: 34. In one embodiment b) consists of SEQ ID NO: 34.

In one embodiment b) comprises SEQ ID NO: 33.

In one embodiment b) comprises at least 75%, 80%, 85%, 90%, 95%, 96%, 97%, 98%, or 99% sequence identity to SEQ ID NO: 33.

In one embodiment b) comprises SEQ ID NO: 33. In one embodiment b) consists essentially of SEQ ID NO: 33. In one embodiment b) consists of SEQ ID NO: 33.

In one embodiment c) comprises SEQ ID NO: 35.

In one embodiment c) comprises at least 75%, 80%, 85%, 90%, 95%, 96%, 97%, 98%, or 99% sequence identity to SEQ ID NO: 35.

In one embodiment c) comprises SEQ ID NO: 10. In one embodiment c) consists essentially of SEQ ID NO: 35. In one embodiment c) consists of SEQ ID NO: 35.

In one embodiment the polynucleotide further comprises d), a first nucleic acid linker linking a) to b), and e) a second nucleic acid linker linking b) to c).

In one embodiment d) or e) or both encode a non-immunogenic linker comprising 1, 2, 3, 4, 5, 6, 7, 8, 9, or 10 amino acid residues, preferably 2, 3 4, 5 or 6, preferably 2, 3, 4 or 5, preferably 3 or 4 amino acid residues.

In one embodiment the amino acid residues encoded by d) or e) or both are small neutral or non-polar amino acid residues.

In one embodiment the small neutral or non-polar amino acid residues are selected from the group consisting of S, G, and A. Preferably the amino acid residues are S or G or both.

In one embodiment d) comprises GGCAGCGGC. In one embodiment d) consists essentially of GGCAGCGGC. In one embodiment d) consists of GGCAGCGGC.

In one embodiment e) comprises SEQ ID NO: 8. In one embodiment e) consists essentially of SEQ ID NO: 8. In one embodiment e) consists of SEQ ID NO: 8.

In one embodiment a) comprises, consists, or consists essentially of SEQ ID NO: 4.

In one embodiment b) comprises, consists, or consists essentially of SEQ ID NO: 6.

In one embodiment c) comprises, consists, or consists essentially of SEQ ID NO: 10.

In one embodiment a) comprises, consists, or consists essentially of SEQ ID NO: 33.

In one embodiment b) comprises, consists, or consists essentially of SEQ ID NO: 34.

In one embodiment c) comprises, consists, or consists essentially of SEQ ID NO: 35.

In one embodiment d) comprises, consists, or consists essentially of GGCAGCGGC.

In one embodiment e) comprises, consists, or consists essentially of SEQ ID NO: 8.

Specifically contemplated as embodiments of this aspect of the invention are embodiments of a) where SEQ ID NO: 6 is replaced with SEQ ID NO: 24, of b) where SEQ ID NO: 6 is replaced with SEQ ID NO: 24 and of c) where SEQ ID NO: 10 is replaced with SEQ ID NO: 26, including embodiments set forth in the previous fusion polypeptide aspects of the invention that are directed, to the nature (including composition and length) and % sequence identity of the amino acid sequences and subsequences identified by SEQ ID NO:, the nature and identity the amino acid residues, including where identified by SEQ ID NO:, and the positioning of these sequences within either a polypeptide or polynucleotide as described herein.

In another aspect the invention relates to a polynucleotide comprising:
a. a first nucleic acid sequence encoding a first portion of the receptor binding domain (RBD) of a wild-type SARS CoV-2 S-protein,
b. a second nucleic acid sequence encoding a second portion of the receptor binding domain (RBD) of a wild-type SARS CoV-2 S-protein,
c. a nucleic acid sequence encoding at least a portion of the N-terminal domain (NTD) of a wild-type SARS CoV-2 protein,
d. a first nucleic acid linker linking a) to b), and
e. a second nucleic acid linker linking b) to c),
   wherein a) and b) are located between the 5′ end of the polynucleotide and c).

In one embodiment a) is comprised by SEQ ID NO: 6. In one embodiment a) comprises at least 95% sequence identity to SEQ ID NO: 6.

In one embodiment a) comprises at least 75%, 80%, 85%, 90%, 95%, 96%, 97%, 98%, or 99% sequence identity to SEQ ID NO: 4.

In one embodiment a) comprises SEQ ID NO: 4. In one embodiment a) consists essentially of SEQ ID NO: 4. In one embodiment a) consists of SEQ ID NO: 4.

In one embodiment b) comprises SEQ ID NO: 6.

In one embodiment b) comprises at least at least 75%, 80%, 85%, 90%, 95%, 96%, 97%, 98%, or 99% sequence identity to SEQ ID NO: 6.

In one embodiment b) comprises SEQ ID NO: 6. In one embodiment b) consists essentially of SEQ ID NO: 6. In one embodiment b) consists of SEQ ID NO: 6.

In one embodiment b) comprises SEQ ID NO: 4.

In one embodiment b) comprises at least 75%, 80%, 85%, 90%, 95%, 96%, 97%, 98%, or 99% sequence identity to SEQ ID NO: 4.

In one embodiment b) comprises SEQ ID NO: 4. In one embodiment b) consists essentially of SEQ ID NO: 4. In one embodiment b) consists of SEQ ID NO: 4.

In one embodiment c) comprises SEQ ID NO: 10.

In one embodiment c) comprises at least 75%, 80%, 85%, 90%, 95%, 96%, 97%, 98%, or 99% sequence identity to SEQ ID NO: 10.

In one embodiment c) comprises SEQ ID NO: 10. In one embodiment c) consists essentially of SEQ ID NO: 10. In one embodiment c) consists of SEQ ID NO: 10.

In one embodiment a) is comprised by SEQ ID NO: 34. In one embodiment a) comprises at least 95% sequence identity to SEQ ID NO: 34.

In one embodiment a) comprises at least 75%, 80%, 85%, 90%, 95%, 96%, 97%, 98%, or 99% sequence identity to SEQ ID NO: 33.

In one embodiment a) comprises SEQ ID NO: 33. In one embodiment a) consists essentially of SEQ ID NO: 4. In one embodiment a) consists of SEQ ID NO: 33.

In one embodiment b) comprises SEQ ID NO: 34.

In one embodiment b) comprises at least at least 75%, 80%, 85%, 90%, 95%, 96%, 97%, 98%, or 99% sequence identity to SEQ ID NO: 34.

In one embodiment b) comprises SEQ ID NO: 34. In one embodiment b) consists essentially of SEQ ID NO: 34. In one embodiment b) consists of SEQ ID NO: 34.

In one embodiment b) comprises SEQ ID NO: 33.

In one embodiment b) comprises at least 75%, 80%, 85%, 90%, 95%, 96%, 97%, 98%, or 99% sequence identity to SEQ ID NO: 33.

In one embodiment b) comprises SEQ ID NO: 33. In one embodiment b) consists essentially of SEQ ID NO: 33. In one embodiment b) consists of SEQ ID NO: 33.

In one embodiment c) comprises SEQ ID NO: 35.

In one embodiment c) comprises at least 75%, 80%, 85%, 90%, 95%, 96%, 97%, 98%, or 99% sequence identity to SEQ ID NO: 35.

In one embodiment c) comprises SEQ ID NO: 10. In one embodiment c) consists essentially of SEQ ID NO: 35. In one embodiment c) consists of SEQ ID NO: 35.

In one embodiment d) or e) or both encode a non-immunogenic linker comprising 1, 2, 3, 4, 5, 6, 7, 8, 9, or 10 amino acid residues, preferably 2, 3 4, 5 or 6, preferably 2, 3, 4 or 5, preferably 3 or 4 amino acid residues.

In one embodiment the amino acid residues encoded by d) or e) or both are small neutral or non-polar amino acid residues.

In one embodiment the small neutral or non-polar amino acid residues are selected from the group consisting of S, G, and A. Preferably the amino acid residues are S or G or both.

In one embodiment d) comprises GGCAGCGGC. In one embodiment d) consists essentially of GGCAGCGGC. In one embodiment d) consists of GGCAGCGGC.

In one embodiment e) comprises SEQ ID NO: 8. In one embodiment e) consists essentially of SEQ ID NO: 8. In one embodiment e) consists of SEQ ID NO: 8.

In one embodiment a) comprises, consists, or consists essentially of SEQ ID NO: 4.

In one embodiment b) comprises, consists, or consists essentially of SEQ ID NO: 6.

In one embodiment c) comprises, consists, or consists essentially of SEQ ID NO: 10.

In one embodiment a) comprises, consists, or consists essentially of SEQ ID NO: 33.

In one embodiment b) comprises, consists, or consists essentially of SEQ ID NO: 34.

In one embodiment c) comprises, consists, or consists essentially of SEQ ID NO: 35.

In one embodiment d) comprises, consists, or consists essentially of GGCAGCGGC.

In one embodiment e) comprises, consists, or consists essentially of SEQ ID NO: 8.

In another aspect the invention relates to a polynucleotide comprising at least 70% nucleic acid sequence identity to a polynucleotide comprising from 5′ to 3′, the following discrete nucleic acid sequences: SEQ ID NO: 4, SEQ ID NO: 4, and SEQ ID NO 10.

In one embodiment, the polynucleotide comprises at least 70% nucleic acid sequence identity to a polynucleotide consisting essentially from 5′ to 3′, of the following discrete nucleic acid sequences: SEQ ID NO: 4, SEQ ID NO: 4, and SEQ ID NO 10.

In one embodiment the polynucleotide comprises at least 75%, 80%, 85%, 90%, 95%, 96%, 97%, 98%, or 99% nucleic acid sequence identity to a polynucleotide comprising from 5′ to 3′, the following discrete nucleic acid sequences: SEQ ID NO: 4, SEQ ID NO: 4, and SEQ ID NO 10.

In one embodiment the polynucleotide comprises at least 75%, 80%, 85%, 90%, 95%, 96%, 97%, 98%, or 99% nucleic acid sequence identity to a polynucleotide consisting essentially, from 5′ to 3′, of the following discrete nucleic acid sequences: SEQ ID NO: 4, SEQ ID NO: 4, and SEQ ID NO 10.

In one embodiment the polynucleotide comprises from 5′ to 3′, the following discrete nucleic acid sequences: SEQ ID NO: 4, SEQ ID NO: 4, and SEQ ID NO 10.

In one embodiment the polynucleotide consists essentially from 5′ to 3′, of the following discrete nucleic acid sequences: SEQ ID NO: 4, SEQ ID NO: 4, and SEQ ID NO 10.

In one embodiment, the polynucleotide comprises at least 70% nucleic acid sequence identity to a polynucleotide comprising from 5′ to 3′, of the following discrete nucleic acid sequences: SEQ ID NO: 6, SEQ ID NO: 4, and SEQ ID NO 10.

In one embodiment, the polynucleotide comprises at least 70% nucleic acid sequence identity to a polynucleotide consisting essentially from 5′ to 3′, of the following discrete nucleic acid sequences: SEQ ID NO: 6, SEQ ID NO: 4, and SEQ ID NO 10.

In one embodiment the polynucleotide comprises at least 75%, 80%, 85%, 90%, 95%, 96%, 97%, 98%, or 99% nucleic acid sequence identity to a polynucleotide comprising from 5′ to 3′, the following discrete nucleic acid sequences: SEQ ID NO: 6, SEQ ID NO: 4, and SEQ ID NO 10.

In one embodiment the polynucleotide comprises at least 75%, 80%, 85%, 90%, 95%, 96%, 97%, 98%, or 99% nucleic acid sequence identity to a polynucleotide consisting essentially, from 5′ to 3′, of the following discrete nucleic acid sequences: SEQ ID NO: 6, SEQ ID NO: 4, and SEQ ID NO 10.

In one embodiment the polynucleotide comprises from 5′ to 3′, the following discrete nucleic acid sequences: SEQ ID NO: 6, SEQ ID NO: 4, and SEQ ID NO 10.

In one embodiment the polynucleotide consists essentially from 5′ to 3′, of the following discrete nucleic acid sequences: SEQ ID NO: 6, SEQ ID NO: 4, and SEQ ID NO 10.

In one embodiment, the polynucleotide comprises at least 70% nucleic acid sequence identity to a polynucleotide comprising from 5′ to 3′, of the following discrete nucleic acid sequences: SEQ ID NO: 4, SEQ ID NO: 6, and SEQ ID NO 10.

In one embodiment, the polynucleotide comprises at least 70% nucleic acid sequence identity to a polynucleotide consisting essentially from 5′ to 3′, of the following discrete nucleic acid sequences: SEQ ID NO: 4, SEQ ID NO: 6, and SEQ ID NO 10.

In one embodiment the polynucleotide comprises at least 75%, 80%, 85%, 90%, 95%, 96%, 97%, 98%, or 99% nucleic acid sequence identity to a polynucleotide comprising from 5′ to 3′, the following discrete nucleic acid sequences: SEQ ID NO: 4, SEQ ID NO: 6, and SEQ ID NO 10.

In one embodiment the polynucleotide comprises at least 75%, 80%, 85%, 90%, 95%, 96%, 97%, 98%, or 99% nucleic acid sequence identity to a polynucleotide consisting essentially, from 5′ to 3′, of the following discrete nucleic acid sequences: SEQ ID NO: 6, SEQ ID NO: 4, and SEQ ID NO 10.

In one embodiment the polynucleotide comprises from 5′ to 3′, the following discrete nucleic acid sequences: SEQ ID NO: 4 SEQ ID NO: 6, and SEQ ID NO 10.

In one embodiment the polynucleotide consists essentially from 5′ to 3′, of the following discrete nucleic acid sequences: SEQ ID NO: 4, SEQ ID NO: 6, and SEQ ID NO 10.

In one embodiment, the polynucleotide comprises at least 70% nucleic acid sequence identity to a polynucleotide comprising from 5′ to 3′, of the following discrete nucleic acid sequences: SEQ ID NO: 6, SEQ ID NO: 6, and SEQ ID NO 10.

In one embodiment, the polynucleotide comprises at least 70% nucleic acid sequence identity to a polynucleotide consisting essentially from 5′ to 3′, of the following discrete nucleic acid sequences: SEQ ID NO: 6, SEQ ID NO: 6, and SEQ ID NO 10.

In one embodiment the polynucleotide comprises at least 75%, 80%, 85%, 90%, 95%, 96%, 97%, 98%, or 99% nucleic acid sequence identity to a polynucleotide comprising from 5′ to 3′, the following discrete nucleic acid sequences: SEQ ID NO: 6, SEQ ID NO: 6, and SEQ ID NO 10.

In one embodiment the polynucleotide comprises at least 75%, 80%, 85%, 90%, 95%, 96%, 97%, 98%, or 99% nucleic acid sequence identity to a polynucleotide consisting essentially, from 5′ to 3′, of the following discrete nucleic acid sequences: SEQ ID NO: 6, SEQ ID NO: 6, and SEQ ID NO 10.

In one embodiment the polynucleotide comprises from 5′ to 3′, the following discrete nucleic acid sequences: SEQ ID NO: 4 SEQ ID NO: 6, and SEQ ID NO 10.

In one embodiment the polynucleotide consists essentially from 5′ to 3′, of the following discrete nucleic acid sequences: SEQ ID NO: 6, SEQ ID NO: 6, and SEQ ID NO 10.

In one embodiment the polynucleotide comprises at least one nucleic acid linker (NLK) as follows:
SEQ ID NO: 4-NLK1—SEQ ID NO: 4-NLK2—SEQ ID NO: 10;
SEQ ID NO: 4-NLK1—SEQ ID NO: 6-NLK2—SEQ ID NO: 10;
SEQ ID NO: 6-NLK1—SEQ ID NO: 4-NLK2—SEQ ID NO: 10;
SEQ ID NO: 6-NLK1—SEQ ID NO: 6-NLK2—SEQ ID NO: 10.

In one embodiment NLK1 or NLK2 or both encode for 1, 2, 3, 4, 5, 6, 7, 8, 9, or 10 amino acid residues, preferably 2, 3 4, 5 or 6, preferably 2, 3, 4 or 5, preferably 3 or 4 amino acid residues.

In one embodiment NLK1 or NLK2 or both encode an amino acid linker. In one embodiment the amino acid linker is a non-immunogenic linker.

In one embodiment NLK1 or NLK2 or both encode a non-immunogenic linker. In one embodiment the non-immunogenic linker comprises small neutral or non-polar amino acid residues.

In one embodiment the small neutral or non-polar amino acid residues are selected from the group consisting of S, G, and A. Preferably the amino acid residues are S or G or both.

In one embodiment a NLK1 comprises, consists, or consists essentially of GGCAGCGGC.

In one embodiment a NLK2 comprises, consists, or consists essentially of SEQ ID NO: 8.

Specifically contemplated as embodiments of the above aspect of the invention related to a polynucleotide comprising at least 70% nucleic acid sequence identity to a polynucleotide comprising from 5′ to 3′, the following discrete nucleic acid sequences: SEQ ID NO: 4, SEQ ID NO: 4, and SEQ ID NO 10, are embodiments in which SEQ ID NO: 4 is replaced by SEQ ID NO: 33, SEQ ID NO: 6 is replaced by SEQ ID NO: 34 and SEQ ID NO: 10 is replaced by SEQ ID NO: 35 including embodiments set forth in the previous fusion polypeptide and polynucleotide aspects of the invention that are directed to the nature (including composition and length) and % sequence identity of the amino and nucleic acid sequences and subsequences identified by SEQ ID NO:, the nature and identity of amino and nucleic acid residues, including where identified by SEQ ID NO:, and the positioning of these sequences within either a polypeptide or polynucleotide as described herein.

In another aspect the invention relates to a polynucleotide comprising at least 70% nucleic acid sequence identity to a polynucleotide comprising at least two discrete copies of SEQ ID NO: 4 and at least one copy of SEQ ID NO: 10.

In one embodiment the polynucleotide comprises at least 70% nucleic acid sequence identity to a polynucleotide consisting essentially of at least two discrete copies of SEQ ID NO: 4 and at least one copy of SEQ ID NO: 10.

In one embodiment the polynucleotide comprises at least 75%, 80%, 85%, 90%, 95%, 96%, 97%, 98%, or 99% nucleic acid sequence identity to a polynucleotide comprising at least two discrete copies of SEQ ID NO: 4 and at least one copy of SEQ ID NO: 10.

In one embodiment the polynucleotide comprises at least 75%, 80%, 85%, 90%, 95%, 96%, 97%, 98%, or 99% nucleic acid sequence identity to a polynucleotide consisting essentially of at least two discrete copies of SEQ ID NO: 4 and at least one copy of SEQ ID NO: 10.

In one embodiment the polynucleotide comprises at least two discrete copies of a nucleic acid sequence comprising at least 70% sequence identity to SEQ ID NO: 4 and at least one discrete copy of a nucleic acid sequence comprising at least 70% sequence identity to SEQ ID NO: 10.

In one embodiment the polynucleotide consists essentially of at least two discrete copies of a nucleic acid sequence comprising at least 70% sequence identity to SEQ ID NO: 4 and at least one discrete copy of a nucleic acid sequence comprising at least 70% sequence identity to SEQ ID NO: 10.

In one embodiment the polynucleotide comprises at least two discrete copies of a nucleic acid sequence comprising at least 75%, 80%, 85%, 90%, 95%, 96%, 97%, 98%, or 99% nucleic acid sequence identity to SEQ ID NO: 4 and at least one discrete copy of a nucleic acid sequence comprising at least 75%, 80%, 85%, 90%, 95%, 96%, 97%, 98%, or 99% nucleic acid sequence identity to SEQ ID NO: 10.

In one embodiment the polynucleotide consists essentially of at least discrete two copies of a nucleic acid sequence comprising at least 75%, 80%, 85%, 90%, 95%, 96%, 97%, 98%, or 99% nucleic acid sequence identity to SEQ ID NO: 4 and at least one discrete copy of a nucleic acid sequence comprising at least 75%, 80%, 85%, 90%, 95%, 96%, 97%, 98%, or 99% nucleic acid sequence identity to SEQ ID NO: 10.

In one embodiment the polynucleotide comprises at least two discrete copies of SEQ ID NO: 4 and at least one discrete copy of SEQ ID NO: 10.

In one embodiment the polynucleotide consists essentially of at least two discrete copies of SEQ ID NO: 4 and at least one discrete copy of SEQ ID NO: 10.

In one embodiment the at least two copies of SEQ ID NO: 4 form a nucleic acid sequence encoding an RBD dimer. In one embodiment the nucleic acid sequence encoding the RBD dimer is positioned between the 5′ end of the polynucleotide and SEQ ID NO: 10.

In one embodiment the polynucleotide further comprises a first nucleic acid linker (NLK1) positioned between the two copies of SEQ ID NO: 4 or a second nucleic acid linker (NLK2) positioned between the two copies of SEQ ID NO: 4 and SEQ ID NO: 10 or both.

In one embodiment the nucleotide comprises at least one nucleic acid linker (NLK) as follows:
SEQ ID NO: 4-NLK1—SEQ ID NO: 4-NLK2—SEQ ID NO: 10;
SEQ ID NO: 4-NLK1—SEQ ID NO: 6-NLK2—SEQ ID NO: 10;
SEQ ID NO: 6-NLK1—SEQ ID NO: 4-NLK2—SEQ ID NO: 10;
SEQ ID NO: 6-NLK1—SEQ ID NO: 6-NLK2—SEQ ID NO: 10.

In one embodiment NLK1 or NLK2 or both encode for 1, 2, 3, 4, 5, 6, 7, 8, 9, or 10 amino acid residues, preferably 2, 3 4, 5 or 6, preferably 2, 3, 4 or 5, preferably 3 or 4 amino acid residues.

In one embodiment NLK1 or NLK2 or both encode an amino acid linker. In one embodiment the amino acid linker is a non-immunogenic linker.

In one embodiment NLK1 or NLK2 or both encode a non-immunogenic linker. In one embodiment the non-immunogenic linker comprises small neutral or non-polar amino acid residues.

In one embodiment the small neutral or non-polar amino acid residues are selected from the group consisting of S, G and A. Preferably the amino acid residues are S or G or both.

In one embodiment a NLK1 comprises, consists, or consists essentially of GGCAGCGGC.

In one embodiment a NLK2 comprises, consists, or consists essentially of SEQ ID NO: 8.

The skilled worker appreciates that SEQ ID NO: 4 is a subsequence of SEQ ID NO: 6. Without wishing to be bound by theory the inventors believe either SEQ ID NO: 4 or SEQ ID NO: 6 (which wholly encompasses SEQ ID NO: 4) or a functional fragment or variant can be used to encode an RBD dimer within a fusion polypeptide as described herein.

Specifically contemplated as embodiments of this aspect of the invention are all embodiments in which the discrete nucleic acid sequences of SEQ ID NO: 4 are substituted for the discrete nucleic acid sequences of SEQ ID NO: 6, in all combinations, including all nucleic acid sequences identified as comprising a specified % of nucleic acid sequence identity.

Accordingly, in an exemplary embodiment the polynucleotide comprises a nucleic acid sequence comprising, consisting, or consisting essentially of discrete copies of SEQ ID NO: 6 and SEQ ID NO: 4, in any order from 5′-3′, linked by an NLK1, this nucleic acid sequence being positioned 5′ to, and linked to SEQ ID NO: 10 in the polynucleotide by a NLK2.

The skilled worker also appreciates that SEQ ID NO: 24 encodes a portion of a SARS CoV-2 S-protein RBD as described herein. Without wishing to be bound by theory the inventors believe that SEQ ID NO: 24 or a functional fragment or variant can be used to encode an RBD dimer within a fusion polypeptide as described herein.

Specifically contemplated as embodiments of this aspect of the invention are all embodiments in which the discrete nucleic acid sequences of SEQ ID NO: 4 or SEQ ID NO: 6 are substituted for the discrete nucleic acid sequences of SEQ ID NO: 24, in all combinations, including all nucleic acid sequences identified as comprising a specified % of nucleic acid sequence identity.

Accordingly, in an exemplary embodiment the polynucleotide comprises a nucleic acid sequence comprising, consisting, or consisting essentially of discrete copies of SEQ ID NO:24 linked 5′-3′ by an NLK1, this nucleic acid sequence being positioned 5′ to, and linked to SEQ ID NO: 26 in the polynucleotide by a NLK2.

Specifically contemplated as embodiments of this aspect of the invention are all embodiments in which the discrete nucleic acid sequences of SEQ ID NO: 4 or SEQ ID NO: 6 are substituted for the discrete nucleic acid sequences of SEQ ID NO: 33 or 34, and the discrete nucleic acid of SEQ ID NO: 10 is substituted for the discrete nucleic acid sequence of SEQ ID NO: 35, in all combinations, including all nucleic acid sequences identified as comprising a specified % of nucleic acid sequence identity.

Accordingly, in an exemplary embodiment the polynucleotide comprises a nucleic acid sequence comprising, consisting, or consisting essentially of discrete copies of SEQ ID NO:33 and 34 linked 5′-3′ by an NLK1, this nucleic acid sequence itself being positioned 5′ to, and linked to a discrete copy of SEQ ID NO: 35 in the polynucleotide by a NLK2.

In another aspect the invention relates to a polynucleotide comprising at least 70% nucleic acid sequence identity to a polynucleotide encoding SEQ ID NO: 1 or a functional fragment or variant thereof.

In one embodiment the polynucleotide consists essentially of a polynucleotide comprising at least 70% nucleic acid sequence identity to a polynucleotide encoding SEQ ID NO: 1 or a functional fragment or variant thereof.

In one embodiment the polynucleotide comprises at least 70% nucleic acid sequence identity to SEQ ID NO: 2.

In one embodiment the polynucleotide consists essentially of a nucleic acid sequence comprising at least 70% nucleic acid sequence identity to SEQ ID NO: 2.

In one embodiment the polynucleotide comprises at least 75%, 80%, 85%, 90%, 95%, 96%, 97%, 98%, or 99% nucleic acid sequence identity to a polynucleotide encoding SEQ ID NO: 1.

In one embodiment the polynucleotide consists essentially of a nucleic acid sequence comprising at least 75%, 80%, 85%, 90%, 95%, 96%, 97%, 98%, or 99% nucleic acid sequence identity to a polynucleotide encoding SEQ ID NO: 1 In one embodiment the polynucleotide comprises at least 75%, 80%, 85%, 90%, 95%, 96%, 97%, 98%, or 99% nucleic acid sequence identity to SEQ ID NO: 2.

In one embodiment the polynucleotide consists essentially of a nucleic acid sequence comprising at least 75%, 80%, 85%, 90%, 95%, 96%, 97%, 98%, or 99% nucleic acid sequence identity to SEQ ID NO: 2.

In one embodiment the polynucleotide comprises a nucleic acid sequence that encodes SEQ ID NO: 1. In one embodiment the polynucleotide consists essentially of a nucleic acid sequence that encodes SEQ ID NO: 1.

In one embodiment the polynucleotide comprises SEQ ID NO: 2. In one embodiment the polynucleotide consists essentially of SEQ ID NO: 2.

In one embodiment, variation in the nucleic acid sequence identity of the polynucleotide as compared to a polynucleotide sequence encoding SEQ ID NO: 1 is variation in the third wobble base position of a codon comprised in the polynucleotide.

In one embodiment the variation does not change the identity of an encoded amino acid.

In one embodiment the variation does not substantially alter the immunogenicity and/or antigenicity and/or ability to be expressed, of SEQ ID NO: 1.

In one embodiment variation in the nucleic acid sequence identity of the polynucleotide as compared to a polynucleotide sequence encoding SEQ ID NO: 1 is variation in the nucleic acid sequences that specify amino acid residues in positions 211-213 and 433-436 of SEQ ID NO: 1.

In one embodiment the variation does not change the identity of an encoded amino acid.

In one embodiment the variation changes the identity of one or more encoded amino acids.

In one embodiment the variation does not substantially alter the immunogenicity and/or antigenicity and/or ability to be expressed, of SEQ ID NO: 1.

In one embodiment variation in the nucleic acid sequences that specify amino acid residues in positions 211-213 and 433-436 of SEQ ID NO: 1 is limited to variation maintains a nucleic acid sequence that encodes for a non-immunogenic amino acid sequence.

In one embodiment the nucleic acid sequences that specify amino acid residues in positions 211-213 and 433-436 of SEQ ID NO: 1 can be the same or different.

In one embodiment they are the same. In one embodiment they are different.

In one embodiment the polynucleotide encodes a non-immunogenic sequence comprising small neutral or small non-polar amino acids. In one embodiment the polynucleotide encodes a non-immunogenic sequence consisting of, or consisting essentially of, small neutral or small non-polar amino acids.

In one embodiment the small neutral or small non-polar amino acids are selected from the group consisting of S, G, and A.

In one embodiment the polynucleotide encodes for amino acid residues GSG at positions 211-213 of SEQ ID NO: 1. In one embodiment the polynucleotide encodes for amino acid residues SGSG at positions 433-436 of SEQ ID NO: 1.

Specifically contemplated as embodiments of this aspect of the invention are all embodiments in which SEQ ID NO: 1 is substituted for SEQ ID NO: 31 and SEQ ID NO: 2 is substituted for SEQ ID NO: 32 in all combinations, including all nucleic acid sequences identified as comprising a specified % of nucleic acid sequence identity.

In another aspect the invention relates to a polynucleotide comprising at least 70% nucleic acid sequence identity to a polynucleotide encoding SEQ ID NO: 21 or a functional fragment or variant thereof.

In one embodiment the polynucleotide consists essentially of a polynucleotide comprising at least 70% nucleic acid sequence identity to a polynucleotide encoding SEQ ID NO: 21 or a functional fragment or variant thereof.

In one embodiment the polynucleotide comprises at least 70% nucleic acid sequence identity to SEQ ID NO: 22.

In one embodiment the polynucleotide consists essentially of a nucleic acid sequence comprising at least 70% nucleic acid sequence identity to SEQ ID NO: 22.

In one embodiment the polynucleotide comprises at least 75%, 80%, 85%, 90%, 95%, 96%, 97%, 98%, or 99% nucleic acid sequence identity to a polynucleotide encoding SEQ ID NO: 21.

In one embodiment the polynucleotide consists essentially of a nucleic acid sequence comprising at least 75%, 80%, 85%, 90%, 95%, 96%, 97%, 98%, or 99% nucleic acid sequence identity to a polynucleotide encoding SEQ ID NO: 21 In one embodiment the polynucleotide comprises at least 75%, 80%, 85%, 90%, 95%, 96%, 97%, 98%, or 99% nucleic acid sequence identity to SEQ ID NO: 22.

In one embodiment the polynucleotide consists essentially of a nucleic acid sequence comprising at least 75%, 80%, 85%, 90%, 95%, 96%, 97%, 98%, or 99% nucleic acid sequence identity to SEQ ID NO: 22.

In one embodiment the polynucleotide comprises a nucleic acid sequence that encodes SEQ ID NO: 21. In one embodiment the polynucleotide consists essentially of a nucleic acid sequence that encodes SEQ ID NO: 21.

In one embodiment the polynucleotide comprises SEQ ID NO: 22. In one embodiment the polynucleotide consists essentially of SEQ ID NO: 22.

In one embodiment, variation in the nucleic acid sequence identity of the polynucleotide as compared to a polynucleotide sequence encoding SEQ ID NO: 21 is variation in the third wobble base position of a codon comprised in the polynucleotide.

In one embodiment the variation does not change the identity of an encoded amino acid.

In one embodiment the variation does not substantially alter the immunogenicity and/or antigenicity and/or ability to be expressed, of SEQ ID NO: 21.

In one embodiment variation in the nucleic acid sequence identity of the polynucleotide as compared to a polynucleotide sequence encoding SEQ ID NO: 21 is variation in the nucleic acid sequences that specify amino acid residues in positions 220 and 440-441 of SEQ ID NO: 21.

In one embodiment the variation does not change the identity of an encoded amino acid.

In one embodiment the variation changes the identity of one or more encoded amino acids.

In one embodiment the variation does not substantially alter the immunogenicity and/or antigenicity and/or ability to be expressed, of SEQ ID NO: 21.

In one embodiment variation in the nucleic acid sequences that specify amino acid residues in positions 220 and 440-441 of SEQ ID NO: 21 is limited to variation maintains a nucleic acid sequence that encodes for a non-immunogenic amino acid sequence.

In one embodiment the nucleic acid sequences that specify amino acid residues in positions 220 and 440-441 of SEQ ID NO: 21 can be the same or different.

In one embodiment they are the same. In one embodiment they are different.

In one embodiment the polynucleotide encodes a non-immunogenic sequence comprising small neutral or small non-polar amino acids. In one embodiment the polynucleotide encodes a non-immunogenic sequence consisting of, or consisting essentially of, small neutral or small non-polar amino acids.

In one embodiment the small neutral or small non-polar amino acids are selected from the group consisting of S, G, and A.

In one embodiment the polynucleotide encodes for amino acid residue G at position 220 or amino acid residues G and S at positions 440-441 or both.

In another aspect the invention relates to an isolated polynucleotide encoding a fusion polypeptide or functional fragment or variant thereof comprising amino acid residues in the following order from the N-terminal of the polypeptide:
a) 319-527 of SEQ ID NO: 19,
b) 319-527 of SEQ ID NO: 19, and
c) 13-290 of SEQ ID NO: 19.

In another aspect the invention relates to an isolated polynucleotide encoding a fusion polypeptide or functional fragment or variant thereof comprising amino acid residues in the following order from the N-terminal of the polypeptide:
a) 319-527 of SEQ ID NO: 19, wherein the amino acid residue at position 452 is an R and the amino acid residue at position 478 is a K,
b) 319-527 of SEQ ID NO: 19, wherein the amino acid residue at position 452 is an R and the amino acid residue at position 478 is a K, and
c) 13-290 of SEQ ID NO: 19, wherein the amino acid residue at position 19 is an R, the amino acid residue at position 142 is a D, and the amino acid residue at position 158 is a G.

Specifically contemplated as embodiments of this aspect of the invention are all of the embodiments set forth in the previous polynucleotide aspects of the invention, particularly but not limited to the identification (SEQ ID NO:), nature (including composition and length) and % sequence identity of the nucleic acid sequences and subsequences identified by SEQ ID NO:, the identification, nature and the amino acid residues coded for by these sequences, including where identified by SEQ ID NO:, and the positioning of these sequences within either a polypeptide or polynucleotide as described herein.

In another aspect the invention relates to an isolated polynucleotide or functional fragment or variant thereof comprising at least 70% nucleic acid sequence identity to a polynucleotide comprising from 5′ to 3′, a) SEQ ID NO 4, b) SEQ ID NO: 4, and c) SEQ ID NO: 10.

In another aspect the invention relates to an isolated polynucleotide or functional fragment or variant thereof comprising at least 70% nucleic acid sequence identity to a polynucleotide comprising from 5′ to 3′, a) SEQ ID NO 26, b) SEQ ID NO: 24, and c) SEQ ID NO: 26.

In another aspect the invention relates to an isolated polynucleotide or functional fragment or variant thereof comprising at least 70% nucleic acid sequence identity to a polynucleotide comprising from 5′ to 3′, a) SEQ ID NO 33, b) SEQ ID NO: 34, and c) SEQ ID NO: 35.

Specifically contemplated as embodiments of this aspect of the invention are all of the embodiments set forth in the previous polynucleotide aspects of the invention, particularly but not limited to the identification (SEQ ID NO:), nature (including composition and length) and % sequence identity of the nucleic acid sequences and subsequences identified by SEQ ID NO:, the identification, nature and the amino acid residues coded for by these sequences, including where identified by SEQ ID NO:, and the positioning of these sequences within either a polypeptide or polynucleotide as described herein.

Also as specifically contemplated as an embodiment of each of the polynucleotide aspects described herein, the polynucleotide can be expressed, or can be designed to be expressed as a vaccine. In one embodiment the polynucleotide is expressed as an mRNA vaccine.

The following polynucleotide embodiments are also specifically contemplated for each of the previous polynucleotide aspects set forth above.

In one embodiment the polynucleotide comprises flanking UTRs, a 5′ cap and a poly(A) tail.

In one embodiment the polynucleotide comprises at least one modified nucleotide residue. In one embodiment the at least one modified nucleotide residue is a pseudo-uridine.

In one embodiment the polynucleotide is a messenger ribonucleic acid (mRNA).

In one embodiment, the polynucleotide is an mRNA molecule comprising a 5′UTR, at least one open reading frame, a 3′UTR, a poly(A) sequence and/or a polyadenylation signal.

In one embodiment the mRNA molecule is codon optimized.

In one embodiment the mRNA molecule is chemically modified. In some embodiments, the chemical modification is 1-methylpseudouridine.

In one embodiment the mRNA is formulated as an mRNA vaccine. In one embodiment the formulation comprises formulation in a nanoparticle, preferably a lipid nanoparticle.

In some embodiments, the lipid nanoparticle comprises a PEG-modified lipid, a non-cationic lipid, a sterol, an ionizable cationic lipid, or any combination thereof.

Formulation of an mRNA vaccine from a polynucleotide as described herein, including formulation in a nanoparticle for therapeutic and/or prophylactic delivery, is believed to be within the skill in the art, for example with reference to U.S. Ser. No. 10/702,600 and/or WO2022/067010, the entireties of which are incorporated by reference herein.

A polynucleotide as described herein, when provided as an mRNA vaccine, encodes a highly immunogenic SARS CoV-2 S-protein antigen(s) capable of eliciting potent antibody and immune cell responses to SARS CoV-2.

In one embodiment, a polynucleotide as described herein is provided as an mRNA vaccine comprising an open reading frame (ORF) that encodes a SARS CoV-2 fusion polypeptide comprising SEQ ID NO: 1 or SEQ ID NO: 31.

In one embodiment the ORF comprises at least 70%, 75%, 80% 85%, 90%, 95% or 99% nucleic acid sequence identity to the nucleic acid sequence of SEQ ID NO: 32.

In one embodiment the ORF comprises, consists essentially of, or consists of, the nucleic acid sequence of SEQ ID NO: 32.

It will be appreciated that the choice of a 5′ UTR and/or 3′ UTR for use in an mRNA vaccine as described herein is believed to be within the skill in the art. Exemplary UTR sequences are provided herein (e.g., SEQ ID NO: 36 and SEQ ID NO: 37) The inventors believe that based on the description of the present application combined with what is known and used in the art, a person of skill in the art can select and use other UTR sequences as known in the art in to fashion an effective mRNA vaccine as described herein (or of no UTR sequences at all in some instances).

In one embodiment the mRNA vaccine comprises a 5′ untranslated region (UTR) comprising the nucleic acid sequence of SEQ ID NO: 36.

In one embodiment the mRNA vaccine comprises a 3′ UTR comprising the nucleic acid sequence of SEQ ID NO: 37.

In some embodiments, the mRNA vaccine disclosed herein comprises an additional structural feature such as a 5′-cap structure or a 3′-poly(A) tail or both.

Addition of a 5′ cap to an mRNA as described herein can be carried out during in vitro-transcription using, as an example, one of the following chemical RNA cap analogs to generate the 5′-guanosine cap structures following the manufacturer protocols: 3<O-Me-m7G(5′)ppp(5′) G [the ARCA cap]; G(5′)ppp(5′)A; G(5′)ppp(5′)G; m7G(5′)ppp(5′)A; m7G(5′)ppp(5′)G (New England BioLabs, Ipswich, MA). 5′-capping of modified RNA may be completed post-transcriptionally using a Vaccinia Virus Capping Enzyme to generate the “Cap 0” structure: m7G(5′)ppp(5′)G (New England BioLabs, Ipswich, MA).

In some embodiments, an mRNA vaccine as described herein comprises a nucleic acid sequence that encodes a signal peptide.

In some embodiments contemplated herein, a signal peptide may have a length of 15-60 amino acids. For example, a signal peptide may have a length of 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 amino acids. In some embodiments, a signal peptide has a length of 20-60, 25-60, 30-60, 35-60, 40-60, 45-60, 50-60, 55-60, 15-55, 20-55, 25-55, 30-55, 35-55, 40-55, 45-55, 50-55, 15-50, 20-50, 25-50, 30-50, 35-50, 40-50, 45-50, 15-45, 20-45, 25-45, 30-45, 35-45, 40-45, 15-40, 20-40, 25-40, 30-40, 35-40, 15-35, 20-35, 25-35, 30-35, 15-30, 20-30, 25-30, 15-25, 20-25, or 15-20 amino acids.

Signal peptides from heterologous genes (which regulate expression of genes other than coronavirus antigens in nature) are known in the art and can be tested for desired properties and then incorporated into a polynucleotide as described herein, including an mRNA.

In one embodiment the signal peptide is a pre prolactin signal peptide that comprises SEQ ID NO: 38. In one embodiment the signal peptide is encoded by SEQ ID NO: 39.

It will be appreciated that the choice of a signal peptide used in an mRNA vaccine as described herein is believed to be within the skill in the art. Exemplary signal peptide sequences provided herein (e.g., SEQ ID NO: 38 and SEQ ID NO: 39) The inventors believe that based on the description of the present application combined with what is known and used in the art, a person of skill in the art can select and use other signal peptide sequences as known in the art in to fashion an effective mRNA vaccine as described herein. For example, GP67 signal peptide, S protein signal peptide MFVFLVLLPLVSSQCV or an immunoglobulin heavy chain variable region (IGVH) signal peptide sequence may be used, but not limited to.

In another aspect the invention relates to an mRNA vaccine comprising from 5′ to 3′, a 5′ UTR, a nucleic acid sequence encoding a signal peptide, SEQ ID NO: 33, SEQ ID NO: 34 and SEQ ID NO: 35, a 3′ UTR and a poly A tail.

In another aspect the invention relates to a transcription unit (TU) comprising at least one polynucleotide as described herein. In one embodiment the TU is comprised in vector, preferably an expression vector. In one embodiment the vector is selected from the group consisting of plasmids, BACs, (PACs), YACs, bacteriophage, phagemids, and cosmids. In one embodiment the vector is a plasmid.

In another aspect the invention relates to a vector comprising a polynucleotide that encodes a fusion polypeptide or functional variant thereof as described herein.

In another aspect the invention relates to a vector comprising a polynucleotide sequence or transcription unit as described herein.

In one embodiment the polynucleotide is comprised in the vector in a TU.

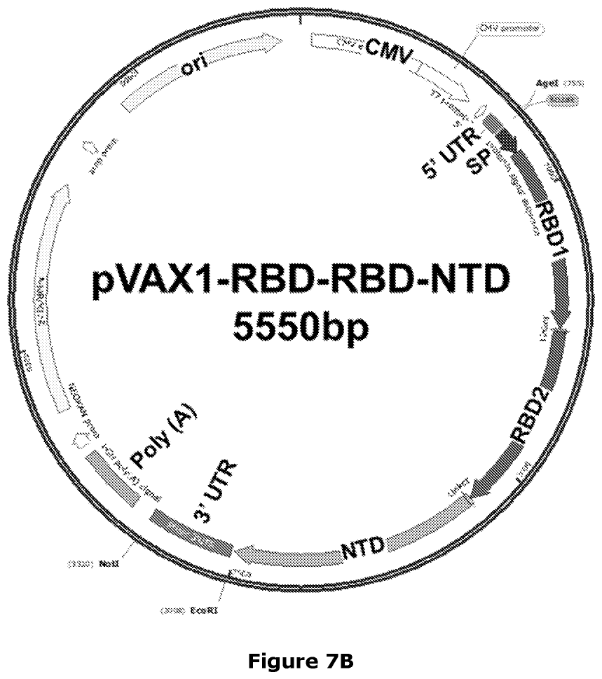

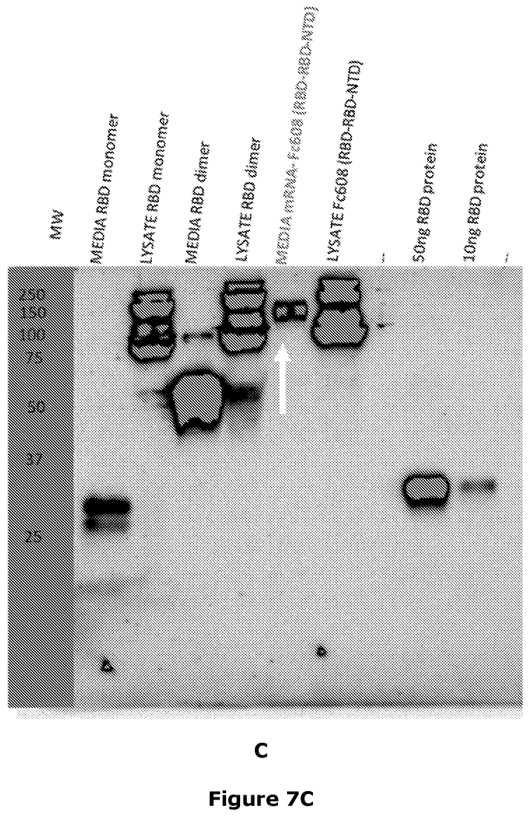

In one embodiment the vector is selected from the group consisting of plasmids, BACs, PACs, YACs, bacteriophage, phagemids, and cosmids. Preferably the vector is a plasmid. In one embodiment the vector is an expression vector. In one embodiment the vector is the pVAX1-fc608-RBD-RBD-NTD plasmid shown in FIG. 7b. In one embodiment the vector comprises SEQ ID NO: 40

A polynucleotide or TU comprising a polynucleotide as described herein can be incorporated into any suitable vector capable of expressing that polynucleotide or, where applicable, an encoded fusion polypeptide as described herein in vitro or in vivo. In one embodiment the vector is an expression vector. In one embodiment the polynucleotide is expressed in vivo a host cell. In one embodiment the polynucleotide is expressed in vitro. In one embodiment the polynucleotide is expressed in vitro in a host cell.

In one embodiment the host cell is an isolated host cell.

The skilled person recognizes that in view of the present disclosure and what is known and used in the art, may suitable vectors be used.

Examples of suitable expression vectors include, but not limited to, plasmid DNA vectors, viral DNA vectors (such as adenovirus and adeno-associated virus), or viral RNA vectors (such as a retroviral vectors). In some embodiments the plasmid and/or phage vectors may be selected from the following vectors or variants thereof including pUC18, pU19, Mp18, Mp19, ColE1, PCR1 and pKRC; lambda gt10 and M13 plasmids such as pBR322, pACYC184, pT127, RP4, p1J101, SV40 and BPV. Also included are vectors such as, but not limited to, cosmids, YACS, BACs shuttle vectors such as pSA3, PAT28 transposons (such as described in U.S. Pat. No. 5,792,294) and the like.

In one embodiment, the vector is pCMV6-XL4 or a functional fragment or variant thereof. In one embodiment the vector is substantially similar to and performs the same function as pCMV6-XL4.

Suitable viral vectors include but are not limited to vectors derived from adenovirus (AV); adeno-associated virus (AAV); retroviruses (e.g., lentiviruses (LV), Rhabdoviruses, murine leukemia virus); herpes virus, and the like. Viral vectors employed herein can be appropriately modified by pseudo typing with envelope proteins or other surface antigens from other viruses, or by substituting different viral capsid proteins, as known, and used in the art.

A transcription unit comprising a polynucleotide as described herein can constructed to drive expression of a fusion polypeptide or polynucleotide, particularly an mRNA, as described herein either in vitro or in vivo. In one embodiment, the TU comprises a polynucleotide as described operatively linked to 5′ or 3′ untranslated regulatory sequences. The design of a particular TU will depend on various factors including the host cells in which the operatively linked polynucleotide is to be expressed and the desired level of polynucleotide expression.

Likewise, the selection of various promoters, enhancers and/or other genetic elements for a TU will depend on various factors including the host cells and expression levels discussed above. In one embodiment, the TU comprises a homologous promoter operatively linked to a polynucleotide as described. In another embodiment, the TU comprises a heterologous promoter operatively linked to a polynucleotide as described. In one embodiment, the homologous or heterologous promoter is an inducible, repressible or regulatable promoter. A suitable promoter may be chosen and used under the appropriate conditions to direct high-level expression of a polynucleotide as described. Many such elements are described in the literature and are available through commercial suppliers.

By way of example only, promoters useful in the expression cassettes can be any suitable eukaryotic or prokaryotic promoter. In one embodiment, the eukaryotic promoter can be a eukaryotic RNA polymerase I (pol I), RNA polymerase II (pol II), or RNA polymerase III (pol III). Expression levels of an operably linked polynucleotide in a particular cell type will be determined by the nearby presence (or absence) of specific gene regulatory sequences (e.g., enhancers, silencers, and the like). Any suitable promoter/enhancer combination (see: Eukaryotic Promoter Data Base EPDB) can be used to drive expression of a polynucleotide as described.

Additional promoters useful in transcription units as contemplated herein include, but are not limited to, β-lactamase, alkaline phosphatase, tryptophan, and tac promoter systems which are all well known in the art. Yeast promoters include 3-phosphoglycerate kinase, enolase, hexokinase, pyruvate decarboxylase, glucokinase, and glyceraldehydrate-3-phosphanate dehydrogenase but are not limited thereto.

Prokaryotic promoters useful in expressing a polynucleotide as described herein from a TU include constitutive promoters as known in the art (such as the int promoter of bacteriophage lamda and the bla promoter of the beta-lactamase gene sequence of pBR322) and regulatable promoters (such as lacZ, recA and gal). A ribosome binding site upstream of the CDS may also be required for expression.

Enhancers useful in a TU include SV40 enhancer, cytomegalovirus early promoter enhancer, globin, albumin, insulin, and the like.

In one embodiment, a TU may be driven by a T3, T7 or SP6 cytoplasmic expression system.

The choice of a particular promoter/enhancer/cell type combination for protein or polynucleotide expression is believed to be within the ordinary skill of those in the art of molecular biology (see, for example, Sambrook et al. (1989) which is incorporated herein by reference).

In another aspect the invention relates to an isolated host cell comprising a fusion polypeptide, isolated polynucleotide, TU and/or isolated vector as described herein.

In one embodiment the isolated host cell is a prokaryotic or eukaryotic cell. Prokaryotes most commonly employed as host cells are strains of Escherichia coli (E. coli). Other prokaryotic hosts include Pseudomonas, Bacillus, Serratia, Klebsiella, Streptomyces, Listeria, Salmonella and Mycobacteria but are not limited thereto.

In one embodiment the eukaryotic cell is an animal cell, a plant cell, a fungal cell, or a protist cell. In one embodiment the animal cell is an insect cell or a mammalian cell, or a cell line of either. In one embodiment the mammalian cell is a Human Embryonic Kidney (HEK), Chinese hamster ovary (CHO), or CV-1 (simian) in Origin, and carrying the SV40 genetic material (COS) cell or cell line.

In another aspect the invention relates to a method of making a fusion polypeptide or polynucleotide as described herein comprising expressing a fusion polypeptide, polynucleotide or TU as described herein in an isolated host cell. In one embodiment the fusion polypeptide, polynucleotide and/or TU are expressed from a vector as described herein. In one embodiment the isolated host cell is in vitro.

In another aspect the invention relates to at least one fusion polypeptide made by a method as described herein.

In another aspect the invention relates to a pharmaceutical composition comprising a fusion polypeptide or polynucleotide as described herein and a pharmaceutically acceptable carrier. In one embodiment the pharmaceutical composition is formulated for administration of the fusion polypeptide or polynucleotide. In one embodiment the polynucleotide is an mRNA vaccine as described herein.

In one embodiment administration comprises systemic administration, preferably parenteral administration. In one embodiment parenteral administration is by injection.

In another aspect the invention relates to a vaccine comprising a fusion polypeptide or polynucleotide as described herein and a pharmaceutically acceptable carrier. In one embodiment the vaccine is a subunit vaccine comprising a fusion polypeptide as described herein and a pharmaceutically acceptable carrier. In one embodiment the vaccine is an mRNA vaccine as described herein and a pharmaceutically acceptable carrier.

In one embodiment the vaccine is formulated for administration of the fusion polypeptide or polynucleotide.

In one embodiment administration comprises systemic administration, preferably parenteral administration. In one embodiment parenteral administration is by injection.

In one embodiment the vaccine further comprises an adjuvant.

In one embodiment the vaccine is formulated for administration with an adjuvant. In one embodiment the vaccine is formulated for separate, simultaneous, or sequential administration with an adjuvant.

In one embodiment the adjuvant is an adjuvant that can be used in pre-clinical trials. In one embodiment the adjuvant is selected from the group consisting of AddaVax (a research grade equivalent of MF59, squalene-Oil-in-water), Seppivac SWE, AddaS03 (a research grade, AS03-like vaccine adjuvant) and Quil-A (a research grade, saponin vaccine adjuvant, combined with MPL-A to generate AS01-like vaccine adjuvant).

In one embodiment the adjuvant is one that is approved for human use. In one embodiment the adjuvant is selected from the group consisting of MF59, Seppivac SWE, AS03, AS01 and Alhydrogel+CpG. In one embodiment the adjuvant is selected from the group consisting of MF59, Seppivac SWE and Alhydrogel+CpG.

In another aspect the invention relates to a kit comprising a fusion polypeptide or polynucleotide as described, a pharmaceutically acceptable carrier and instructions for administration.

In another aspect the invention relates to a method of treating or preventing a SARS CoV-2 infection in a subject comprising administering to the subject a therapeutically effective amount of a fusion polypeptide, polynucleotide, pharmaceutical composition, or vaccine as described herein.

Specifically contemplated as embodiments of this aspect of the invention, the polynucleotide is an mRNA vaccine as described herein.

In one embodiment administration comprises systemic administration, preferably parenteral administration. In one embodiment parenteral administration is by injection.

In one embodiment administration further comprises administration with an adjuvant. In one embodiment, administration of the adjuvant is separate, simultaneous, or sequential administration.

In one embodiment administration comprises administering a unit dose of about 10 ug to about 200 ug of the polynucleotide or mRNA. In one embodiment the unit dose is from about 15 ug to about 150 ug, about 20 ug to about 125 ug, or about 30 ug to about 100 ug, preferably about 30 ug to 100 ug of the polynucleotide or mRNA. In one embodiment the unit dose comprises about 30 ug to 50 ug of the polynucleotide or mRNA. In one embodiment the unit dose comprises about 50 ug to about 100 ug of the polynucleotide or mRNA.

In one embodiment administration comprises administering a unit dose of about 10 ug, about 20 ug, 30 ug, 40 ug, 50 ug, 60 ug, 70 ug, 80 ug, 90 ug, 100 ug, 110 ug, 120 ug, 130 ug, 140 ug, 150 ug, 160 ug, 170 ug, 180 ug, 190 ug, about 200 ug of the polynucleotide or mRNA. In one embodiment the unit dose comprises about 30 ug, about 50ug or about 100 ug of the polynucleotide or mRNA.

In one embodiment the unit dose is a prime dose. In one embodiment the unit dose is a booster dose.

In one embodiment administration comprises administering a prime dose followed a first pre-determined time later with a booster dose. In one embodiment administration comprises a second booster dose at a second pre-determined time after the administration of the first booster dose.

In one embodiment first pre-determined time is from 3 to 16 weeks.

In one embodiment the second pre-determined time is from 3 to 16 weeks.

In one embodiment the mRNA is encapsulated in a lipid nanoparticle.

Additionally, specifically contemplated as embodiments of the above method of treatment aspect of the invention are all of the embodiments set forth previously in the aspects of the invention relating to the fusion polypeptides, polynucleotides, compositions and vaccines as described herein, including embodiments set forth in the previous fusion polypeptide and polynucleotide aspects of the invention that are directed to the nature (including composition and length) and % sequence identity of the amino and nucleic acid sequences and subsequences identified by SEQ ID NO:, the nature and identity of amino and nucleic acid residues, including where identified by SEQ ID NO:, and the positioning of these sequences within either a polypeptide or polynucleotide as described herein. In another aspect the invention relates to a fusion polypeptide, polynucleotide, pharmaceutical composition, or vaccine as described herein for use treating or preventing a SARS CoV-2 infection in a subject.

In another aspect the invention relates to the use of a fusion polypeptide, polynucleotide, pharmaceutical composition, or vaccine as described herein in the manufacture of a medicament for treating or preventing a SARS CoV-2 infection in a subject.

In one embodiment the medicament comprises an effective amount of the fusion polypeptide, polynucleotide, pharmaceutical composition, or vaccine. In one embodiment the vaccine is an mRNA vaccine as described herein.

In one embodiment the effective amount is a therapeutically effective amount.

In one embodiment the medicament is formulated for administration, or is in a form for administration, to a subject in need thereof.

In one embodiment the medicament is in a form for, or is formulated for, parenteral administration of the fusion polypeptide, polynucleotide, pharmaceutical composition, or vaccine. In one embodiment parenteral administration is injection.

In one embodiment the medicament is formulated for, or is in the form of an injectable composition, or when administered, is administered by injection.

In one embodiment the medicament is in a form for, or is formulated for, parenteral administration in any appropriate solution, preferably in a sterile aqueous solution which may also contain buffers, diluents, and other suitable additives.

In one embodiment the medicament is in a form for, or is formulated for, use with an adjuvant. In one embodiment the use with the adjuvant is separate, simultaneous, or sequential use.

Specifically contemplated as embodiments of the above use and for use aspects of the invention are all of the embodiments set forth in the aspects of the invention relating to a method of treating or preventing a SARS CoV-2 infection in a subject comprising administering to the subject a therapeutically effective amount of a fusion polypeptide, polynucleotide, pharmaceutical composition, or vaccine as described herein, including all of the specific embodiments directed to the fusion polypeptides, polynucleotides, compositions and vaccines as set out specifically herein.

In this specification where reference has been made to patent specifications, other external documents, or other sources of information, this is generally for the purpose of providing a context for discussing the features of the invention. Unless specifically stated otherwise, reference to such external documents is not to be construed as an admission that such documents; or such sources of information, in any jurisdiction, are prior art, or form part of the common general knowledge in the art.

The invention will now be illustrated in a non-limiting way by reference to the following examples.

### EXAMPLES

### Materials and Methods—General

### Example-1: Polynucleotide Expression

### Gene Synthesis

The cDNA sequence of each SARS-CoV-2 construct was synthesized by Gene Universal (Geneuniversal.com) and cloned by 5′ NotI and 3′ XbaI into a modified pCMV6-XL4 vector in frame with an N-terminal FLAG and C-terminal human Fc fragment.

### Example-2: Polypeptide Expression and Characterization

### HEK293 Cell Culture

HEK293S cells were cultured in DMEM supplemented with 5% foetal bovine serum at 37° C. and 5% CO₂. When culturing stable cell lines, HEK293 cells lacking N-acetylglucosaminyltransferase I (GnTI) activity (HEK293 GnTI−) were obtained from American Type Culture Collection (ATCC, Manassas, VA). Cells were maintained in DMEM supplemented with 5% FBS and 500 μg/ml of G418 at 37° C. and 5% CO₂.

Stable cell lines were made for all constructs by selecting against geneticin resistance. Cells were selected in DMEM supplemented with 5% FBS and 500 μg/ml of G418. Resistant clones were isolated using Pyrex cloning rings and stable expression of the proteins were tested via western blot. Clones that highly expressed the protein were amplified, frozen, and used in large-scale protein production.

### Protein Expression and Purification

Fc-608 and other fusion polypeptides as described herein were concentrated and purified from cell culture media as follows. Fc-antigens (e.g.) HEK293 cells were transfected using the polyethylenimine method (Longo et al. 2013) with the cDNA encoding for the 608 polypeptides fused to the Fc region of human IgG together with an empty vector plasmid (pcDNA3.1) conferring G418 resistance. Forty-eight hours after transfection, cells were selected in DMEM supplemented with 5% FBS and 500 μg/mL G418. Resistant cell clones were isolated using Pyrex cloning rings (Corning) and stable expression of the antigen protein was tested via western blot. The best expressing clones were amplified, frozen and used for large scale protein production.

Large-scale protein production was performed in cell culture flasks with regular collection and replenishment of cell culture medium containing 2-5% FBS. Secreted proteins were purified by affinity chromatography, using ANTI-FLAG M2 Affinity resin (Sigma, #A2220). The saturated resin was washed (50 mM Tris pH 7.4, 450 mM NaCl), equilibrated (50 mM Tris pH 7.4, 150 mM NaCl) and the protein eluted (50 mM Tris pH 7.4, 150 mM NaCl, FLAG peptide 100 μg/mL).

Fc-608 was purified using Protein-A Sepharose 4 fast flow resin (Cytiva). The saturated resin was washed (50 mM Tris pH 7.4, 450 mM NaCl), equilibrated (50 mM Tris pH 7.4, 150 mM NaCl, 1 mM DTT), and the protein was eluted (50 mM Tris pH 7.4, 150 mM NaCl, 1 mM DTT, 10 mg/ml HRV-3C protease) by cleaving the protein at an HRV-3C protease site engineered between the C-terminal of the protein and the start of the Fc region.

The purified antigens were concentrated to 10 μM using a Vivaspin concentrator (Sartorius-Stedim) and flash-frozen with liquid nitrogen and stored at −80° C. until required.

The protein samples were further concentrated and used immediately or flash-frozen in liquid nitrogen and stored at −80° C. until needed.

### Analytical Procedures

SDS-PAGE Coomassie staining were performed using standard procedures. Protein concentrations were estimated by measuring the absorbance at 280 nm (data not shown).

Size exclusion chromatography: aggregation, oligomerization or other in-solution behaviour was monitored by size exclusion chromatography using either a Superose 6 Increase 10/300 GL or a HiLoad 16/600 Superdex 200 PG column equilibrated in 20 mM HEPES pH 7.4, 150 mM NaCl, 1 mM CaCl₂).

### Bio-Layer Interferometry

BLI experiments were performed at room temperature on a BLItz instrument (ForteBio). Protein A biosensors were pre-wetted in 400 μl of 20 mM HEPES pH 7.4, 150 mM NaCl, 1 mM CaCl₂), 0.2% Tween-20, 0.1% bovine serum albumin (BSA) for 10 min before use. The Protein A biosensors were then incubated for 4-10 min to load the appropriate purified Fc-fusion protein (e.g., ACE2-Fc or RBD-Fc). The binding event took place in a 4 μl drop of purified protein at a series of concentrations, under agitation. The lengths of the association and dissociation steps were determined empirically so that signal returned to baseline when possible. When sensible, the BLI experiments were performed in triplicate or duplicate.

### ELISA

An ELISA-based assay was used to test the binding between RBD-Fc and ACE2-AP fusions. A solution at 3 μg/mL of mouse anti-AP in 1×PBS was added to each well of 96-well plates using an automated multichannel pipette, sealed, and incubated overnight at 4 C. The following day, plates were washed, and 1% casein was added as a blocking agent, which was removed after 1 hr at room temperature using an automated microplate washer. Subsequently, to each well, 20 ml of ecto-AP conditioned medium containing 2 mL of monoclonal mouse anti-human IgG1-HRP (2 μg/mL) was added using an automated plate copier along with 20 ml of ecto-Fc culture medium. Plates were sealed and incubated for 4 hr at room temperature in the dark. Plates were subsequently washed, and 15 mL 1-Step Ultra TMB-ELISA HRP substrate was added using an automated multichannel pipette; after 1 hr incubation at room temperature, the absorbance at 650 nm was recorded with a microplate plate reader. Finally, plates were scanned to obtain matching images of the 650 nm reading. Positive controls were used to gauge the sensitivity of the assay and negative controls were used to obtain background values to quantify the positive reactions.

### Example-3: Non-Expressing Fusion Polynucleotide Constructs

The following expression constructs were designed to produce fusion polypeptides comprising amino acid sequences from the RBD and NTD of the wild-type SARS CoV-2 S-protein.

### Fusion Polypeptide Fc-604

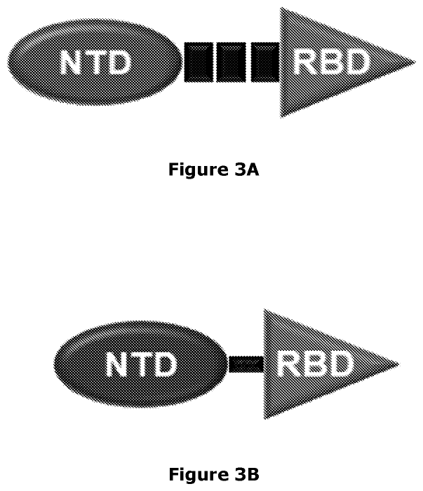

This fusion polypeptide was expressed from a polynucleotide comprising SEQ ID NO: 16, was designed to comprise a portion of the amino acid sequence of the RBD of the wild-type SARS CoV-2 S-protein located between the N-terminus of the polypeptide and the amino acid sequence of the NTD of the wild-type SARS CoV-2 S-protein (FIG. 3). Fc-604 is distinguished from Fc-608 and Fc-609 in comprising the entire NTD+RBD and intervening sequences.

The amino acid sequence of the unpurified Fc-604 fusion polypeptide:

(SEQ ID NO: 15)
MDSKGSSQKGSRLLLLLVVSNLLLCQGVVSDYKDDDDKAAAGSGSLEVLF
QGPSSQCVNLTTRTQLPPAYTNSFTRGVYYPDKVFRSSVLHSTQDLFLPF
FSNVTWFHAIHVSGTNGTKRFDNPVLPFNDGVYFASTEKSNIIRGWIFGT
TLDSKTQSLLIVNNATNVVIKVCEFQFCNDPFLGVYYHKNNKSWMESEFR
VYSSANNCTFEYVSQPFLMDLEGKQGNFKNLREFVFKNIDGYFKIYSKHT
PINLVRDLPQGFSALEPLVDLPIGINITRFQTLLALHRSYLTPGDSSSGW
TAGAAAYYVGYLQPRTFLLKYNENGTITDAVDCALDPLSETKCTLKSFTV
EKGIYQTSNFRVQPTESIVRFPNITNLCPFGEVFNATRFASVYAWNRKRI
SNCVADYSVLYNSASFSTFKCYGVSPTKLNDLCFTNVYADSFVIRGDEVR
QIAPGQTGKIADYNYKLPDDFTGCVIAWNSNNLDSKVGGNYNYLYRLFRK
SNLKPFERDISTEIYQAGSTPCNGVEGFNCYFPLQSYGFQPTNGVGYQPY
RVVVLSFELLHAPATVCGPKKSTNLVKNKCVNFNFNGLTGTGVLTESNKK
FLPFQQFGRDIADTTDAVRDPQTLEILDITPCSFGGVSVITPGTNTSNQV
AVLYQDVNCTEVPVAIHADQLTPTWRVYSTGSNVFQTRAGCLIGAEHVNN
SYECDIPIGAGICASYQTQTNSPALEVLFQGPDPDPEEPKSCDKTHTCPP
CPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKAL
PAPIEKTISKAKGQPREPQVYTLPPSRDELTKNQVSLTCLVKGFYPSDIA
VEWESNGQPENNYKTTPPVLDSDGSFFLYSKLTVDKSRWQQGNVFSCSVM
HEALHNHYTQKSLSLSPGK.

With reference to SEQ ID NO: 15, Fc-604 comprises
a. a prolactin leader peptide (residues 1-30)
b. an N-terminal DYKDDDDK epitope (residues 31-38) fused to
c. residues 39-45 fused to
d. an HRV 3C protease recognition site (residues 46-53) fused to
e. an amino acid sequence of the N-terminal domain (NTD) of SARS CoV-2 wild-type S-protein (residues 54-332) fused to
f. amino acid residues (333-360) fused to
g. an amino acid sequence of the receptor binding domain (RBD) of the SARS CoV-2 wild-type S-protein (residues 361 to 579) fused to
h. amino acid residues 580 to 724 fused to
i. an HRV 3C protease recognition site (residues 725-732) fused to
j. a human Fc sequence (residues 733-737).

Fc-604 did not express at detectable levels (data not shown).

### Fusion Polypeptide Fc-609

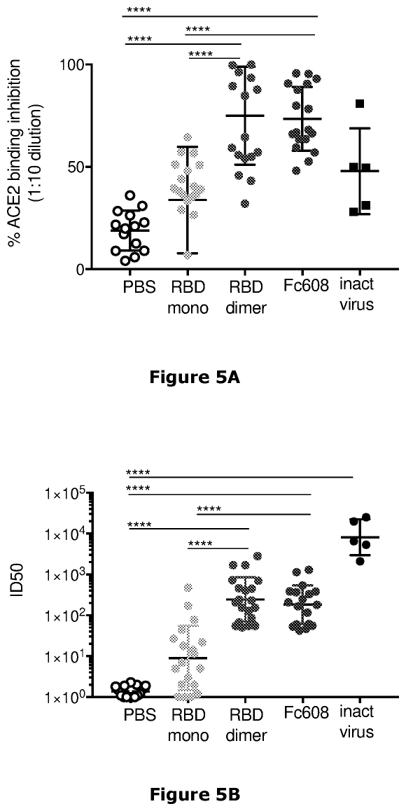

This fusion polypeptide expressed from a polynucleotide comprising SEQ ID NO: 18 was designed to comprise a portion of the amino acid sequence of the RBD of the wild-type SARS CoV-2 S-protein located between the N-terminus of the polypeptide and the amino acid sequence of the NTD of the wild-type SARS CoV-2 S-protein (FIG. 5).

The amino acid sequence of the unpurified Fc-609 fusion polypeptide.

(SEQ ID NO: 17)
MDSKGSSQKGSRLLLLLVVSNLLLCQGVVSDYKDDDDKAAALEVLFQGPS
SQCVNLTTRTQLPPAYTNSFTRGVYYPDKVFRSSVLHSTQDLFLPFFSNV
TWFHAIHVSGTNGTKRFDNPVLPFNDGVYFASTEKSNIIRGWIFGTTLDS
KTQSLLIVNNATNVVIKVCEFQFCNDPFLGVYYHKNNKSWMESEFRVYSS
ANNCTFEYVSQPFLMDLEGKQGNFKNLREFVFKNIDGYFKIYSKHTPINL
VRDLPQGFSALEPLVDLPIGINITRFQTLLALHRSYLTPGDSSSGWTAGA
AAYYVGYLQPRTFLLKYNENGTITDAVDGSGSRFPNITNLCPFGEVFNAT
RFASVYAWNRKRISNCVADYSVLYNSASFSTFKCYGVSPTKLNDLCFTNV
YADSFVIRGDEVRQIAPGQTGKIADYNYKLPDDFTGCVIAWNSNNLDSKV
GGNYNYLYRLFRKSNLKPFERDISTEIYQAGSTPCNGVEGFNCYFPLQSY
GFQPTNGVGYQPYRVVVLSFELLHAPATVCGPKKSTNLVKNSALEVLFQG
PDPDPEEPKSCDKTHTCPPCPAPELLGGPSVFLFPPKPKDTLMISRTPEV
TCVVVDVSHEDPEVKFNWYVDGVEVHNAKTKPREEQYNSTYRVVSVLTVL
HQDWLNGKEYKCKVSNKALPAPIEKTISKAKGQPREPQVYTLPPSRDELT
KNQVSLTCLVKGFYPSDIAVEWESNGQPENNYKTTPPVLDSDGSFFLYSK
LTVDKSRWQQGNVFSCSVMHEALHNHYTQKSLSLSPGK.

With reference to SEQ ID NO: 17, Fc-609 comprises
a. a prolactin leader peptide (residues 1-30)
b. an N-terminal DYKDDDDK epitope (residues 31-38) fused to
c. residues 39-41 fused to
d. an HRV 3C protease recognition site (residues 42-49) fused to
e. an amino acid sequence of the N-terminal domain (NTD) of SARS CoV-2 wild-type S-protein (residues 50-328) fused to
f. amino acid residues (329-332) fused to
g. an amino acid sequence of the receptor binding domain (RBD) of the SARS CoV-2 wild-type S-protein (residues 333 to 541) fused to
h. amino acid residues 542-543 fused to
i. an HRV 3C protease recognition site (residues 544-551) fused to
j. a human Fc sequence (residues 552-556).

Fc-609 did not express at detectable levels (data not shown).

### Example 4: SARS-CoV2 Immunogenicity Study Methods

### Immunisations

C57BL/6 mice are immunised intramuscularly in the hind limb. Each mouse is immunised with 50 μg of protein vaccine candidate, or 6 μg of inactivated virus. Each immunisation is in a 100 μL volume; 50 μL of 1 mg/mL protein, or 12 mg/mL virus, in PBS; admixed with 50 μL of Addavax. Negative control mice are immunised similarly with 100 μL PBS. Half the vaccine (50 μL volume) is immunised into each hind limb. Groups of 10 mice/group are immunized twice, spaced 3 weeks apart (day 0 and day 21). 1 week after the last boost (day 28) mice are sacrificed and blood and spleen are collected for analysis. Serum from blood samples were assessed for anti-RBD IgG, anti-RBD IgG affinity, surrogate virus neutralization test (SVNT), and replication-competent SARS-CoV-2 neutralization assay. T cell responses from the spleen are assessed by IFNg ELISpot following restimulation with peptide mixes derived from SARS-CoV2 Spike S1 and S2 domain, Spike RBD or nuclear capsid protein.

### ELISAS

### Conventional ELISA

Thermo Scientific Nunc MicroWell 96-Well Microplates plates are coated with 100 μL of 2 μg/mL receptor binding domain (RBD) (yeast produced) in carbonate buffer overnight at 4° C. Plates are washed five times with 200 μL per well of PBS+0.05% Tween. Plates are blocked for 1 hour at RT with 200 μL per well of PBS+10% Gibco heat inactivated foetal calf serum (FCS). 1.5 μL of test serum is added to 148.5 μL of PBS+10% FCS for a 1:100 starting dilution; this is done in duplicate in adjacent columns. Diluted test serum is diluted serially diluted 1:5, down plate rows, in 10% FCS. Controls: four wells per plate are left blank (10% FCS only), for normalising; four wells per plate have 1:100 PBS treated serum, to find end point; one column per plate has serial dilution of monoclonal mouse anti-spike IgG1 Clone 43 antibody (Cat: 40591-MM43) starting concentration 10 ng/mL. Blocking buffer is removed and 100 μL diluted antibody solution is added and incubated for 2 hours at room temperature. Plates are again washed five times with 200 μL per well of PBS+0.05% Tween. Plates are incubated with 1 μg/mL Invitrogen goat anti-mouse total IgG HRP (ref A10551), diluted in PBS+10% FCS for 1 hour at RT. Plates are again washed five times with 200 μL per well of PBS+0.05% Tween. Plates are treated with BD OptEIA TMB substrate reagent set (100 μL per well 1:1 substrate A and B), and reaction is stopped with 50 μL 2M H₂SO₄. The reaction is read at 450 nm on a Perkin Elmer EnSpire 2300 multilabel plate reader. End point titres are the titres at which the sample crosses the threshold of the “end point”; the “end point” is calculated as the average signal of the 1:100 PBS treated serum, plus four times the standard deviation of those reads.

### Surrogate Neutralisation Assay

Thermo Scientific Nunc MicroWell 96-Well Microplates plates are coated with 100 μL of 2 μg/mL receptor binding domain (RBD) (yeast produced) in carbonate buffer overnight at 4° C. Plates are washed five times with 200 μL per well of PBS+0.05% Tween. Plates are blocked for 1 hour at RT with 200 μL per well of PBS+10% Gibco heat inactivated foetal calf serum (FCS). 6 μL of test serum is added to 54 μL of PBS+10% FCS for a 1:10 starting dilution; this is done in duplicate in adjacent columns. Diluted test serum is diluted serially diluted 1:4, down plate rows, in 20% FCS. Controls: two columns are left blank (20% FCS only). Blocking buffer is removed and 50 μL diluted antibody solution is added and incubated for 2 hours at room temperature. Without washing in between steps, 50 μL of 10% FCS with 40 ng/mL hACE-Fc (2×Kd) is added, and plates are incubated for 2 hours at RT. Control: columns previously left blank is also omitted from treatment with hACE2-Fc. Plates are again washed five times with 200 μL per well of PBS+0.05% Tween. Plates are incubated with 1 μg/mL Biorad anti-human Fc HRP (ref MCA647P), diluted in PBS+10% FCS for 1 hour at RT. Plates are again washed five times with 200 μL per well of PBS+0.05% Tween. Plates are treated with BD OptEIA TMB substrate reagent set (100 μL per well 1:1 substrate A and B), and reaction is stopped with 50 μL 2M H₂SO₄. The reaction is read at 450 nm on a Perkin Elmer EnSpire 2300 multilabel plate reader. The IC50 is reported as the titre at which 50% of the uninhibited signal is read.

### Affinity Assay

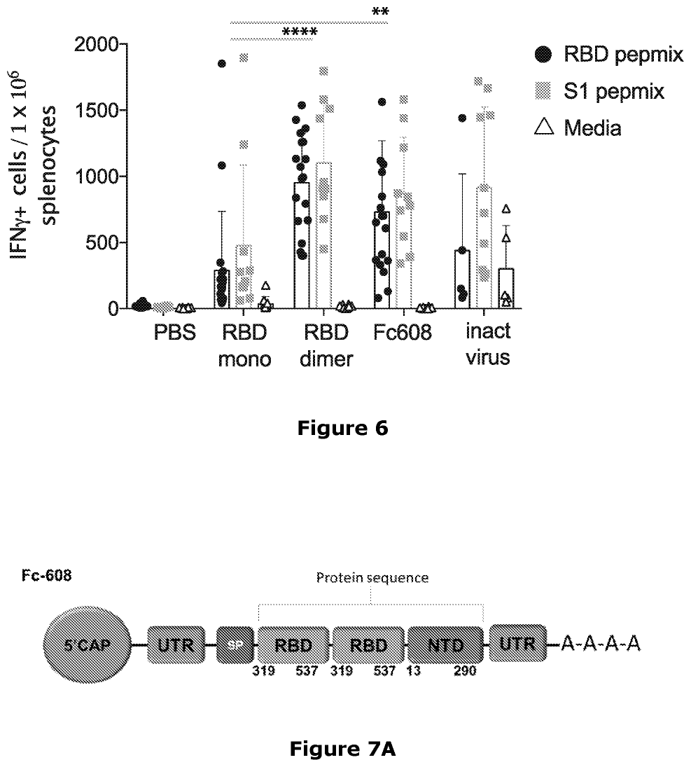

Thermo Scientific Nunc MicroWell 96-Well Microplates plates are coated with 100 μL of 2 μg/mL receptor binding domain (RBD) (yeast produced) in carbonate buffer overnight at 4° C. Plates are washed five times with 200 μL per well of PBS+0.05% Tween. Plates are blocked for 1 hour at RT with 200 μL per well of PBS+10% Gibco heat inactivated foetal calf serum (FCS). 1.5 μL of test serum is added to 148.5 μL of PBS+10% FCS for a 1:100 starting dilution; this is done in double duplicate, one duplicate in adjacent columns, and then a second duplicate six columns over, such that columns 1-6 are identical to columns 7-12. Diluted test serum is diluted serially diluted 1:5, down plate rows, exclude row H in 10% FCS. Controls: row H is left blank (10% FCS only), for normalising. Blocking buffer is removed and 100 μL diluted antibody solution is added and incubated for 2 hours at room temperature. Plates are again washed five times with 200 μL per well of PBS+0.05% Tween. PBS is added to rows 1:6; 6M urea in PBS is added to rows 7-12, plates are incubated at 37° C. for 30 minutes. At end of incubation plates are immediately doused with PBS+0.05% tween to stop the reaction. Plates are again washed five times with 200 μL per well of PBS+0.05% Tween. Plates are incubated with 1 μg/mL Invitrogen goat anti-mouse total IgG HRP (ref A10551), diluted in PBS+10% FCS for 1 hour at RT. Plates are again washed five times with 200 μL per well of PBS+0.05% Tween. Plates are treated with BD OptEIA TMB substrate reagent set (100 μL per well 1:1 substrate A and B), and reaction is stopped with 50 μL 2M H₂SO₄. The reaction is read at 450 nm on a Perkin Elmer EnSpire 2300 multilabel plate reader. The affinity index is reported as the signal for the urea treated sample, as a percentage of the untreated signal, at the dilution at which the untreated signal is at 80% of the max signal. This is selected as the “dilution of interest” to ensure we are assessing loss of signal as the “linear” section of the sigmoidal curve, and not when the antibody is saturating (FIG. 6?)

### T Cell IFNγ ELISpot

Splenocytes were plated at 300,000 cells per well into wells of a pre-coated mouse IFNγ capture ELISpot plate (Mabtech 3321-4AST-10) along with either peptide mixes, media control or PMA/ionomycin control. Peptide mixes are from JPT and are selected based on the vaccine used to immunize mice (i.e., RBD, whole spike protein or NCAP protein). Plates are incubated overnight at 37° C. and cytokine producing cells are detected using the antibodies/SA-ALP in the ELISpot kit.

### Neutralization Assay Method

Patient-derived SARS-COV-2 isolates were propagated and titrated by TCID₅₀ on Vero-E6 expressing TMPRSS2 (Matsuyama et al., 2020). Serum derived from mice vaccinated with SARS-CoV-2 spike proteins and convalescent serum from SARS-CoV-2 patients were heat-treated at 56° C. for 30 minutes, then diluted 1:10 and titrated 1:5 in DMEM containing 10% heat-inactivated FBS and 1 mg/ml Geneticin, then incubated with 0.02 moi/cell SARS-CoV-2 for 1 hour. Vero-E6-TMPRSS2 cells were then inoculated with the mixture and incubated at 5% CO₂ and 37° C. for 72 hours. Wells were scored for CPE and a BCA assay performed to assess neutralizing effects of the sera on the virus.

### Example-5: SARS-CoV2 Immunogenicity Study Results

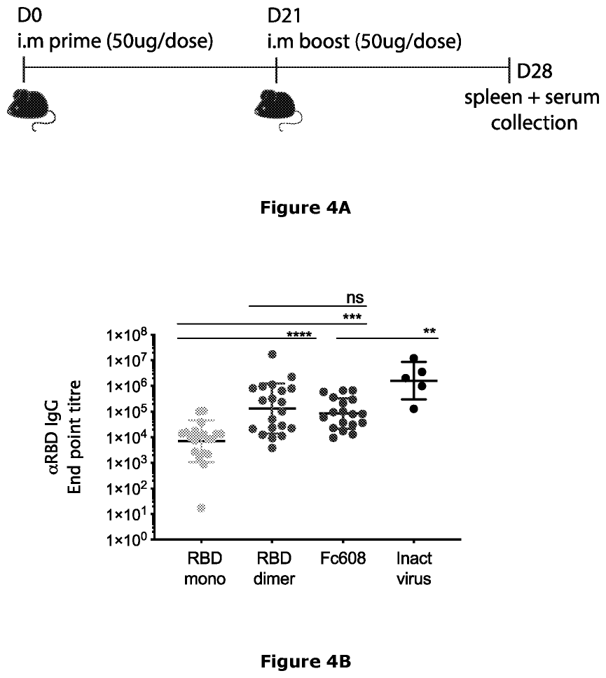

The immunogenicity of Fc608 adjuvanted with AddaVax was assessed and compared to RBD monomer and dimer constructs using a prime/boost regimen spaced 21 days apart (FIG. 4A). Groups of C57BL/6 mice (n=10/group) were immunized intramuscularly with 50 ug of the different RBD protein constructs or 6 ug of formalin inactivated SARS-CoV-2 virus in equal volume with Addavax and control mice were treated with PBS. Anti-RBD IgG antibodies were detected in the blood 7 days after booster immunization of all vaccinated mice compared to PBS. Mice immunized with Fc608, RBD dimer or inactivated SARS-CoV-2 virus had similar antibody titres and were significantly higher (63-fold or greater) compared to mice that received RBD monomer subunit vaccine (FIG. 4B).

Similarly, in comparison to immunization with RBD monomer, mice immunized with Fc608, RBD dimer or inactivated SARS-CoV-2 virus, had higher titer antibodies that neutralised the cytopathic effect (CPE) of SARS-CoV-2 on Vero Temprss2 cells and blocked hACE2 receptor binding to RBD in a surrogate neutralization test (SVNT) (FIG. 5).

To assess the antigen-specific T cell response elicited by RBD protein subunit vaccines and inactivated SARS-COV-2, cells isolated from the spleen at day 7 following booster immunization were measured for IFNg in an ELISpot assay (FIG. 6). Spleen cells were restimulated with RBD peptide mix consisting of a pool of 53 15mer peptides or Spike subunit 1 pepmix containing a pool of 166 peptides incorporating both RBD and N-terminal domain. The number of IFNg secreted cells after re-stimulation increased 2-fold or higher in spleens of mice immunized with Fc608, RBD Dimer compared to RBD Monomer (FIG. 6). Of note, immunization with inactivated SARS-CoV-2 promote a higher proportion of T cell specific for antigens outside of the RBD.

### Example-6: SARS-CoV2 Immunogenicity in K-18 Mice—Study Results

Immunization with Fc608 provides Protection Equivalent to that Seen in Convalescent Mice

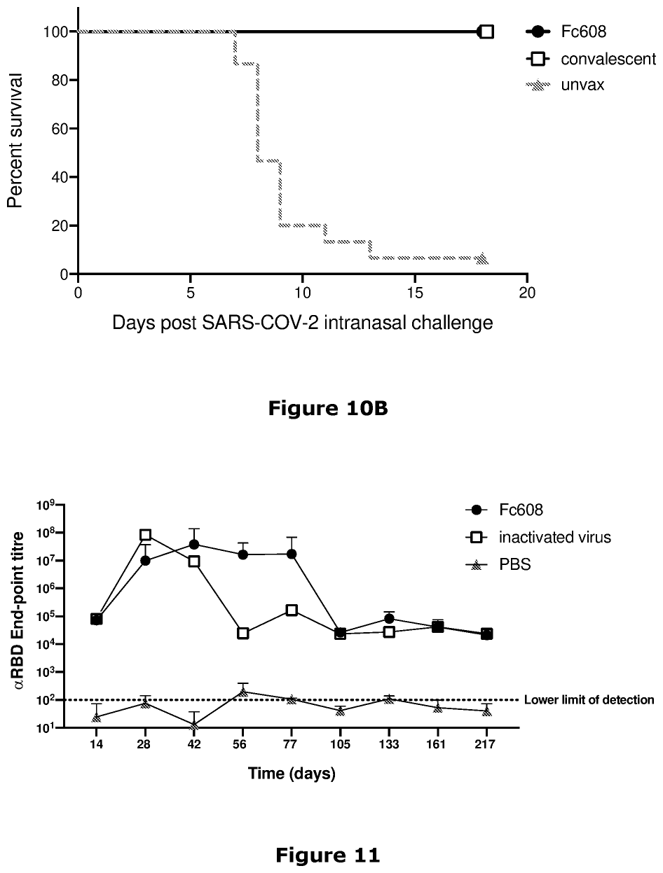

The protective capacity of Fc608 was assessed in K-18 mice, which are highly susceptible to infection with SARS-CoV-2. K-18 mice carry the human ACE2 protein on the keratin 18 promotor, as such they express ACE2 on epithelial cells, including the respiratory mucosa. Mice were immunized on day 0 and 21 with PBS or 50 mg of Fc608 either with or without Addavax™. As a positive control, a convalescent group of mice was established, these mice received a low dose inoculation of 10² SARS-CoV-2 TCID₅₀ (WA/2020) on day 14. All groups of mice were intranasally challenged with 5×10³ TCID₅₀ of SARS-CoV-2 (WA/2020) on Day 35 and monitored for body weight changes and mortality (FIG. 10a). Like convalescent mice, all mice immunized with Fc608+Addavax™ were protected from SARS-CoV-2 infection and did not lose body weight (FIG. 10a). In contrast, unvaccinated mice rapidly lost weight and by day 10 all but one had succumbed to infection (FIG. 10b).

Immunization with Fc608 Induces Long-Lived Antibodies Equivalent to that Seen in Convalescent Mice

To assess the durability of the antibody response initiated by Fc608, we tracked α-RBD IgG titers over time, following immunization. Antibody titers from vaccinated mice were compared to titers from mice treated with PBS, or formalin-inactivated SARS-CoV-2 virus, which represents an approved COVID-19 vaccine format currently administered to millions worldwide. Antibody levels in vaccinated mice peaked at approximately 70 days after the first dose and then plateaued at 100 days after the second dose, remaining stable for at least 220 days (FIG. 11).

### Immunity Generated by Fc628 (Delta Version) is Comparable to Fc608 (Wuhan Version)

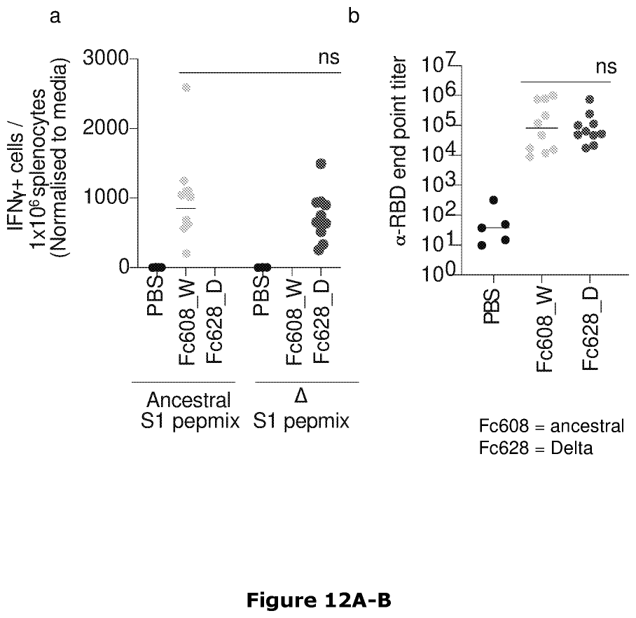

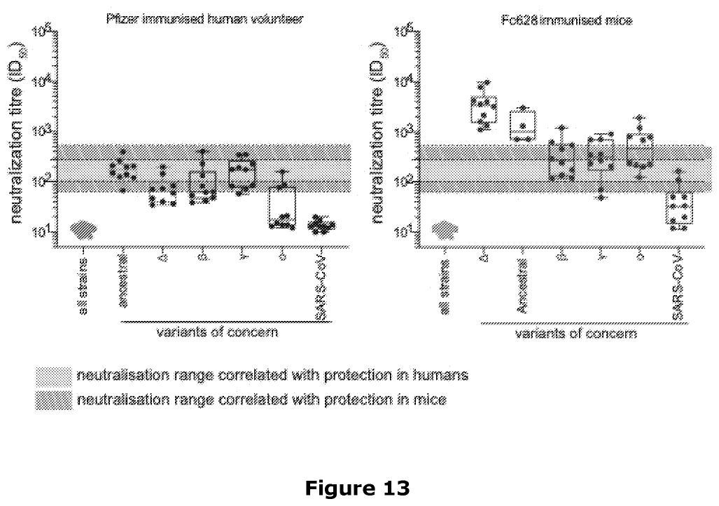

To address the emergence of the highly transmissible Delta variant, we developed a new RBD-RBD-NTD construct based on this variant, which we refer to as Fc628. We first compared the immunogenicity of the ancestral- and Delta-based constructs. Groups of C57BL/6 mice were immunized on day 0 and 21 with AddaVax™ and 50 mg of Fc608 or Fc628 or treated with PBS as a negative control. To assess the T cell response, on day 28 IFNg ELISpot assays were performed on splenocytes restimulated with overlapping peptide pools from the S1 domain of the respective variants. Immunization with either ancestral or Delta-based RBD-RBD-NTD protein induced similar number of IFNγ-producing T cells specific for S1 (FIG. 12a). Likewise, both protein constructs induced similar titers of RBD binding antibodies (FIG. 12b).

Immunization with Fc628 Provides Cross Protection Against SARS-CoV-2 Variants of Concern

A successful booster vaccine should provide protective cross-reactive antibodies that can confer protection against multiple variants of concern as well as future variants. To assess the cross-reactivity of Fc628, we generated a pseudotyped lentivirus panel that included lentiviruses that express the spike glycoprotein from SARS-CoV-1 or SARS-CoV2 variants (Delta, Beta, Gamma, Omicron). We first measured the protective capacity of human-vaccinated antibody responses against the different variants (FIG. 13). In order to benchmark NAb responses in humans and mice, we compared titers against a protective range, which was an indicator of NAb titer necessary to achieve protection. Given that BNT162b2 provides 95% protection against ancestral SARS-CoV-2 infection, the protective range for human vaccinated sera (FIG. 13) was determined as the NAb titer against ancestral strain. Similarly, K18-ACE2 mice vaccinated with Fc608 were completely protected from live SARS-COV-2 intranasal challenge (FIG. 10a, 10b). Therefore, the protective range for mouse vaccinated sera was set at the level of NAb titer against ancestral strain from mice immunized with Fc608. mRNA vaccines encoding ancestral strain FL-Spike neutralized against Beta, Delta and Gamma with reduced efficiency compared to neutralization against the ancestral strain (FIG. 13). Based on the protective range we have set, serum from vaccinated individuals were only protective against ancestral and the Gamma SARS-CoV-2 variant (FIG. 13). Neutralizing efficacy against the highly divergent Omicron strain was considerably lower, with 70% of human samples falling outside of the protective range. In contrast, we found that mice immunized with Fc628 had high levels of cross protective antibodies against Omicron, Beta and Gamma SARS-CoV2. We also found a marginal increase in NAb titers for SARS-CoV in vaccinated mice compared to NAb titers from PBS controls (FIG. 13).

### Incorporation of the NTD Enhances Immunogenicity of the RBD Subunit Vaccine

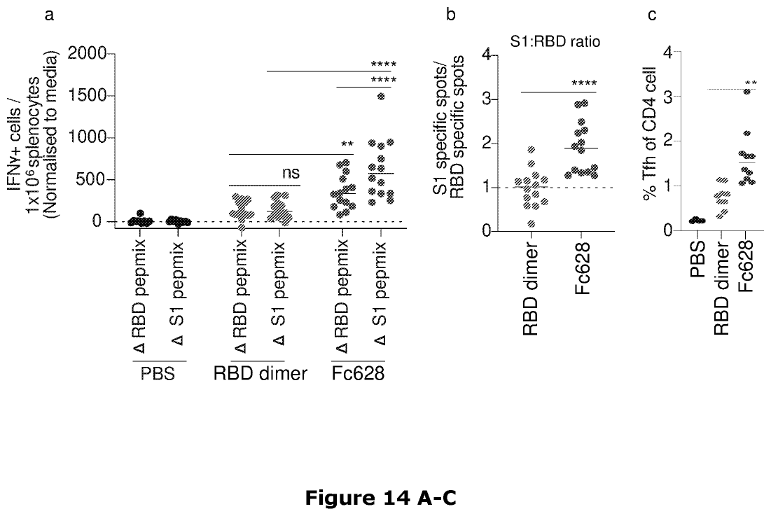

To determine whether the addition of the NTD within Fc628 resulted in an increased breadth of T cell epitopes used, we compared the antigen-specific response to vaccination with a Delta variant RBD dimer. T cell responses against the different regions of spike protein were assessed by restimulation with overlapping peptide pools from RBD or S1 (RBD+NTD). When mice were immunized with RBD dimer, there was no significant difference between the number of RBD-specific or S1-specific IFNγ producing T cells. In contrast, immunization with Fc628 resulted in the expansion of S1 specific T cells that was significantly greater than RBD-specific T cells (FIG. 14a), where the difference was most likely due to T cells specific for the NTD. Furthermore, the ratio of S1 to RBD spot forming units revealed that the contribution of the NTD-specific T cells was 2-fold greater than the RBD-specific T cell response (FIG. 14b).

Given that vaccination with Fc628 promoted enhanced T cell immunity, we sought to determine whether a higher proportion of T follicular helper (Tfh) cells was induced. Tfh cells are critical for the germinal center (GC) response and support B cell activation, class switching and affinity maturation. Expression levels of the Tfh-associated molecules B-cell lymphoma 6 protein (Bcl6) and Programmed death-1 (PD-1) were assessed on CD4+ T cells from the draining lymph node (DLN). Notably, the proportion of Tfh CD4+ cells detected in mice vaccinated with Fc628 was significantly greater than what was found in RBD dimer vaccinated mice (FIG. 14c).

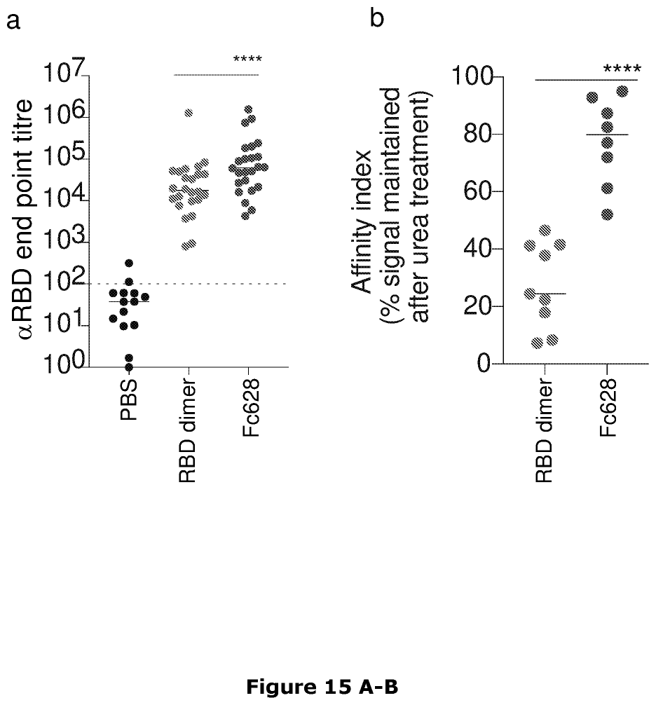

Next we determined whether the addition of the NTD in the Fc628 protein improved the antibody response to RBD. Immunization with VAANZ-D_RRN induced higher levels of anti-RBD IgG antibodies (FIG. 15a) Affinity (FIG. 15b) compared to titers in mice immunized with the RBD dimer protein.

### Example-7: Translation of Fc608 RBD Subunit Fusion Protein into an mRNA Vaccine Format

mRNA-Fc608 encodes a fusion protein of a receptor binding domain (RBD) tandem dimer with the N-terminal domain of the Spike protein fused to the C-terminus (RBD-RBD-NTD). The complete polynucleotide sequence of the 608 mRNA is SEQ ID NO: 32. SEQ ID NO: 32 contains a mammalian Pre pro-lactin signal peptide fused to the N-terminus. The 3′ and 5′ untranslated region sequences are adapted from the Pfizer COVID-19 mRNA vaccine (FIG. 7a). Antigens were designed from the Spike sequence of SARS-CoV-2 Wuhan strain and cloned into pVAX-1 vector (FIG. 7b).

### Methods

### Gene Synthesis

The cDNA insert of RBD-RBD-NTD SARS-CoV-2 construct was codon optimized for human cell expression, synthesized and sequence verified by Gene Universal (Geneuniversal.com) in to a pVAX1 expression vector.

mRNA Synthesis and Lipid Nanoparticle Encapsulation

Plasmid DNA was purified and linearized with XbaI. In vitro transcription was performed using HiScribe™ T7 High Yield RNA Synthesis Kit (E2040) by NEB in the presence of N1-Methylpseudo-UTP instead of standard UTPs. The product was LiCl purified and a cap1 structure was added by incubation with Vaccinia Capping System (M2080) by NEB and mRNA Cap 2′-O-Methyltransferase (M0366) by NEB. The product was LiCl purified and a poly A tail was added using E. coli Poly (A) Polymerase (M0276) by NEB. The final product was LiCl purified, taken up in Sodium Acetate buffer, and stored at −80 C until encapsulation. Integrity of the RNA was confirmed by electrophoresis using a Tapestation. mRNA was encapsulated into lipid nanoparticles (LNP) by mixing with GenVoy Ionizable Lipid Mix using an Ignite NanoAssemblr. The LNPs had a diameter of ˜100 nm, as measured by dynamic light scattering using a Zetasizer Nano ZS instrument. Concentration of encapsulated mRNA was determined by Ribogreen assay. mRNA-LNPs were stored at 4 C ready for injection into mice.

### Mice

Specific pathogen-free mice were bred and housed at the Malaghan Institute of Medical Research. C57BL/6J mouse breeding pairs were originally obtained from the Jackson Laboratory. Sex-matched mice between 6-11 weeks of age were used for all experiments and mice were age-matched within 2 weeks of each other in any given experiment. All experimental protocols were approved by the Victoria University of Wellington Animal Ethics Committee and experiments were carried out in accordance with their guidelines.

### Immunizations

Mice were immunized by intramuscular injection on both legs, each injection containing 50 μL per leg. Mice were immunised twice, three weeks apart, and both immunizations included either 5 μg Fc608-mRNA LNP or 50 μg RBD dimer protein with AddaVax™ adjuvant (Invivogen, cat #: vac-adx-10) per mouse.

### Tissue Preparation and Cell Isolation

Seven days following the secondary immunization, mice were sacrificed, and blood, spleens, and both inguinal lymph nodes (iLNs) were collected for analysis. Blood was obtained via cardiac puncture, collected into Microvette® serum gel tubes (Sarstedt, cat #: SARS20.1344) before centrifugation at 10,000×g for 5 minutes and serum was decanted. Single cell suspensions of splenocytes were prepared by mashing spleens through a 70 μM cell strainer (Falcon, cat #: BDAA352350) with the end of a 3 mL syringe (BD, cat #: 302100) and washing through with Iscove's Modified Dulbecco's Medium (IMDM) (Gibco, cat #: 31980-097) before centrifugation at 250×g for 10 minutes. Cell pellets were resuspended in red blood cell lysis solution (Qiagen, cat #: 158904) and cells were centrifuged at 1600 RPM for 4 minutes before resuspension in R10 media (Roswell Park Memorial Institute [RPMI]1640 Medium [Gibco, cat #: 11875-119] supplemented with heat inactivated 10% fetal bovine serum [FBS][Gibco, cat #: 10091-148]) and filtered through a 70 μM cell strainer. Single cell suspensions of iLNs were prepared by mashing iLNs through a 70 μM cell strainer with the end of a 1 mL syringe (BD, cat #: 302113) and washing through with IMDM. Cells were then centrifuged at 250×g for 10 minutes before resuspension in R10.

### Conventional ELISA

Nunc MaxiSorp™ 96-Well ELISA Microplates (ThermoFisher, cat #: 442404) were coated with 100 μL of 2 μg/mL receptor binding domain (RBD) in carbonate buffer overnight at 4° C. Plates were washed five times with 200 μL per well of PBS+0.05% Tween. Plates were blocked at RT for 1 hour with 200 μL per well of PBS+10% FBS. 1.5 μL of test serum was added to 148.5 μL of PBS+10% FBS for a 1:100 starting dilution; this was done in duplicate in adjacent columns. Diluted test serum was serially diluted 1:5, down plate rows, in 10% FBS. For control samples, four wells per plate were left blank (10% FBS only) for normalizing; four wells per plate had 1:100 PBS treated serum to find the endpoint; and one column per plate had serially diluted monoclonal mouse anti-spike IgG1 Clone 43 antibody (SinoBiological, Cat #: 40591-MM43), at a starting concentration 10 ng/mL. Blocking buffer was removed and 100 μL diluted antibody solution was added and incubated at RT for 2 hours. Plates were again washed five times with 200 μL per well of PBS+0.05% Tween. Plates were incubated with 1 μg/mL goat anti-mouse total IgG HRP (Invitrogen, cat #: G21040), diluted in PBS+10% FBS at RT for 1 hour. Plates were again washed five times with 200 μL per well of PBS+0.05% Tween. Plates were treated with OptEIA™ TMB substrate reagent set (100 μL per well 1:1 substrate A and B [BD, cat #: 555214]), and the reaction was stopped with 50 μL 2M H₂SO₄. The reaction was read at 450 nm on a Perkin Elmer EnSpire 2300 multilabel plate reader. End point titers are the titers at which the sample crosses the threshold of the “end point”; the “end point” was calculated as the average signal of the 1:100 PBS treated serum, plus four times the standard deviation of those reads.

### Pseudotyped Virus Neutralization Assay

The ability of mouse or human serum samples to neutralize SARS-CoV-2 spike-mediated entry was determined. Briefly, HEK293/ACE2 cells were seeded in poly-D-lysine coated, white-walled, 96-well plates (20,000 cells/well) and incubated at 37° C., 5% CO₂ for 24 hrs. Serum samples collected from immunized mice or COVID-19 convalescent patients were heat-treated at 56° C. for 30 min, diluted with cell culture medium (1:10, then 1:5 serial dilutions), mixed with a suspension of the SARS-CoV-2 spike pseudotyped lentiviral particles (enough to generate >1,000-fold signal over background, approximately 3 to 4×10⁵ relative light units [RLU]/well) in 96-well plates at a 1:1 ratio (150 μl final volume) and incubated at 37° C., 5% CO₂ for one hour. The cell culture media of the HEK293/ACE2 cells was removed, replaced with the mixture of serially diluted serum with SARS-CoV-2 spike pseudotyped lentiviruses, plus 5 μg/ml of polybrene (Sigma-Aldrich Merck), and incubated at 37° C., 5% CO₂ for 72 hrs. Viral entry was quantified by removing the cell culture supernatant and adding a 1:1 mixture of fresh cell culture media (50 μl) and luciferin reagent (50 μl, Steady-Luc Firefly assay kit, Biotium, Fremont, CA) to each well. Plates were incubated at room temperature with gentle shaking (300 rpm) for 5 min and luminescence measured using a plate reader (VICTOR Nivo, PerkinElmer, Waltham, MA). Neutralizing antibody titers were calculated by a non-linear regression model (log inhibitor vs. normalized response-variable slope) analysis and expressed as 50% neutralizing titer (NT₅₀).

### Interferon Gamma (IFNγ) ELISpot

Plates pre-coated with mAb AN18 (Mabtech, cat #: 3321-3-250) were washed and conditioned with R10 media according to the manufacturer's protocol. Splenocytes were plated at 3×10⁵ cells per well in R10 containing PepMix™ peptide pools (JPT, cat #: PM-WCPV-S-1 and #: PM-WCPV-S-RBD-1) at a final concentration of 0.5 μg/mL. Negative control wells (media only) and positive control wells containing PMA at 2.5 ng/mL and ionomycin at 1 μg/mL were included for each sample. Plates were incubated at 37° C. for 18 hours before development according to the manufacturer's protocol. Spots were enumerated using an AID ELISpot reader and software. To generate normalized readings, spots in negative controls were subtracted from corresponding test wells and counts were presented as spot forming units per million splenocytes.

### In Vitro Expression of RBD-RBD-NTD Protein

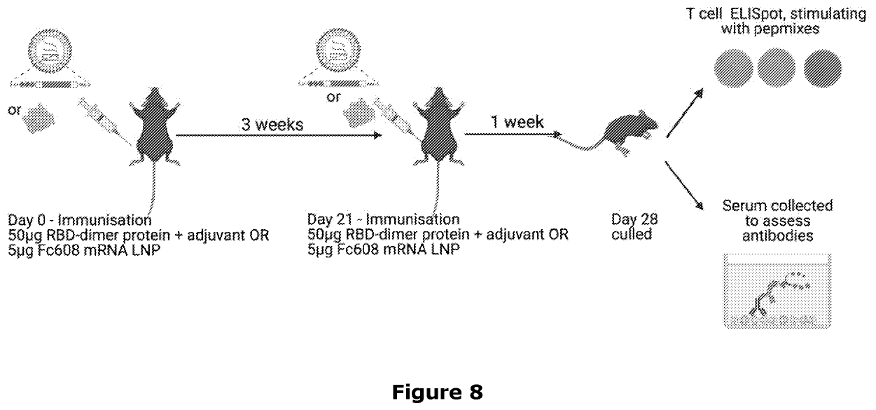

HEK293T cells were transfected with 2.5 μL of Expifectamine (Thermo) transfection reagent mixed with 1 μg DNA encoding mRNA-608 (SEQ ID NO: 32) or RBD-monomer (SEQ ID NO: 5) or RBD-Dimer (SEQ ID NO: 11) and incubated for 4 days. Culture supernatant and lysate from transfection media was collected and run on a 12% SDS PAGE. Proteins were transferred to nitrocellulose membrane and detected with a polyclonal antibody that recognises SARS-CoV-2 RBD. Expression of Fc608 protein was compared to purified RBD monomer. Western blot confirms the expression of a protein product at the expected size ˜90 kDa (FIG. 8).

### Immunogenicity Studies in Mice

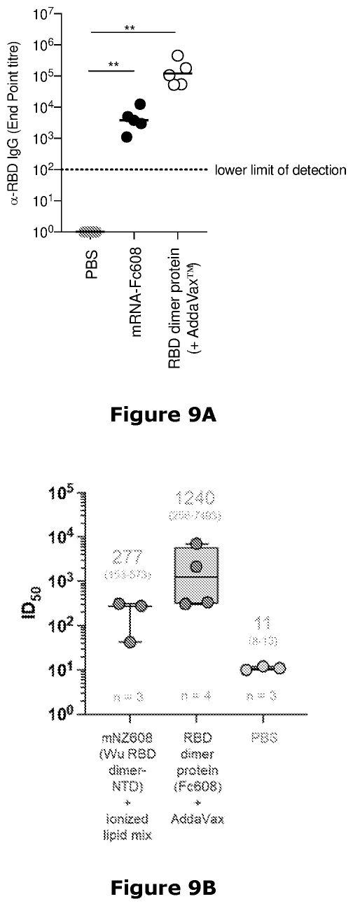

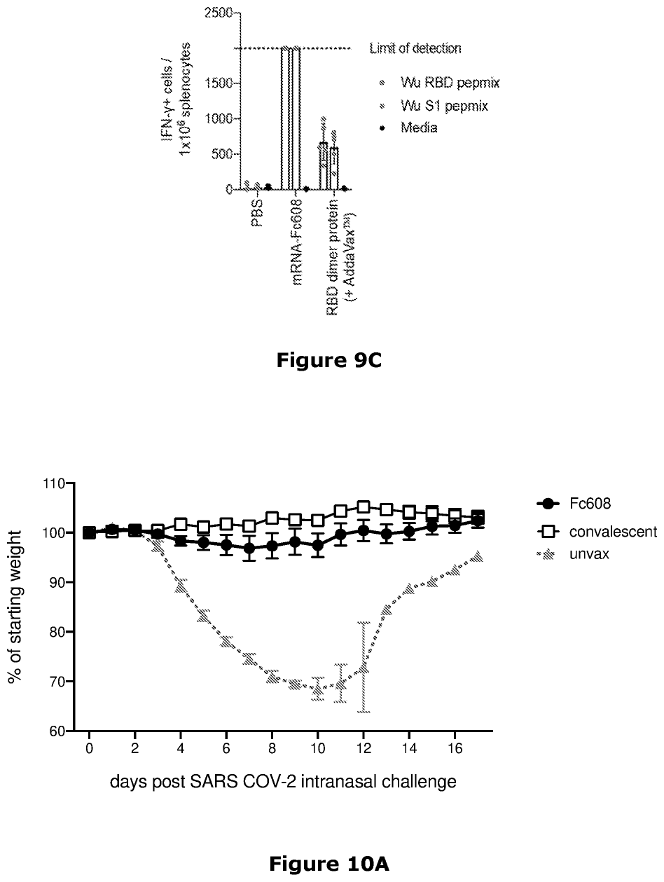

Immunisation with Fc608-mRNA LNPs was assessed in mice using a prime/boost regimen spaced 21 days apart (FIG. 9). Groups of C57BL/6 mice (n=5/group) were immunized intramuscularly with 5 mg/dose of Fc608-mRNA LNP, 50 mg/dose of RBD dimer protein mixed in equal volume with AddaVax or PBS. Anti-RBD IgG antibodies were detected in the blood 7 days after booster immunisation of vaccinated mice compared to PBS. Mice immunized with Fc608-mRNA LNPs or RBD dimer had significantly higher titers compared to mice that received PBS (FIG. 10a).

Neutralising antibody titers were determined in a Pseudovirus neutralisation assay. Serum from vaccinated and PBS control mice were incubated with SARS-CoV-2 Spike (ancestral strain) Pseudovirus, prior to culture with HEK293/hACE2 cells. Serum from Fc608-mRNA LNP or RBD dimer vaccinated mice had higher titer antibodies that neutralised the cytopathic effect compared to serum from PBS treated controls (FIG. 10b).

To assess the antigen-specific T cell response elicited by Fc608-mRNA-LNP and RBD dimer vaccines, cells isolated from the spleen at day 7 following booster immunization were measured for IFNg in an ELISpot assay. Spleen cells were restimulated with RBD peptide mix consisting of a pool of 53 15mer peptides or Spike subunit 1 pepmix containing a pool of 166 peptides incorporating both RBD and N-terminal domain. The number of IFNg secreted cells after re-stimulation increased significantly in spleens of mice immunized with Fc608-mRNA-LNP, compared to RBD Dimer protein and PBS treated controls. (FIG. 10c).

### INDUSTRIAL APPLICATION

The invention has industrial application in the production of a vaccine for use in treating and/or preventing SARS CoV-2 infection and/or for use in the manufacture of medicaments for the treatment and/or prevention of SARS CoV-2 infection.

### REFERENCES

1. Hu, B., Guo, H., Zhou, P. et al. Characteristics of SARS-CoV-2 and COVID-19. Nat Rev Microbiol 19, 141-154 (2021).
2. Longo P A, Kavran J M, Kim M S, Leahy D J. Transient mammalian cell transfection with polyethylenimine (PEI). Methods Enzymol. 2013; 529:227-240.

1. A fusion polypeptide or functional fragment or variant thereof comprising at least one SARS CoV-2 Spike protein (S-protein) receptor binding domain dimer (RBD dimer) fused to at least a portion of a SARS CoV-2 N-terminal domain (NTD) by a non-immunogenic amino acid linker, wherein the RBD dimer is located between the N-terminus of the fusion polypeptide and the portion of the NTD.

2. The fusion polypeptide of claim 1 wherein the RBD dimer comprises two copies of SEQ ID NO: 3 or a functional fragment or variant thereof.

3. The fusion polypeptide of claim 1 wherein the RBD dimer comprises any one of SEQ ID NOs: 5, 23, or 27, or a functional fragment or variant thereof.

4. (canceled)

5. The fusion polypeptide of claim 1 wherein the RBDs in the RBD dimer are fused to each other in a tandem repeat.

6. The fusion polypeptide of claim 1 wherein the RBDs in the RBD dimer are fused to each other by a non-immunogenic amino acid linker.

7. (canceled)

8. The fusion polypeptide of claim 1 wherein the RBD dimer comprises at least 95% amino acid sequence identity to SEQ ID NO: 11.

9. (canceled)

10. (canceled)

11. The fusion polypeptide of claim 1 wherein the N-terminal domain (NTD) comprises SEQ ID NO: 9 or SEQ ID NO: 25.

12. (canceled)

13. The fusion polypeptide of claim 1 wherein the RBD dimer and the NTD are fused to each other by a non-immunogenic amino acid linker.

14. (canceled)

15. The fusion polypeptide of claim 1 comprising SEQ ID NO: 1, wherein the amino acid residues at positions 211-213 and 432-436 are small neutral or small non-polar amino acid residues.

16. The fusion polypeptide of claim 1 comprising SEQ ID NO: 21, wherein the amino acid residues at positions 220 and 440-441 are small neutral or small non-polar amino acid residues.

17. A fusion polypeptide or functional fragment or variant thereof comprising in the following order from the N-terminus to the C-terminus, peptide domains (a), (b) and (c), wherein
(a) is a portion of the receptor binding domain (RBD) of the SARS CoV-2 S-protein,
(b) is a portion of the receptor binding domain (RBD) of the SARS CoV-2 S-protein, and
(c) is a portion of the N-terminal domain (NTD) of the SARS CoV-2 S-protein.

18. The fusion polypeptide of claim 17 wherein (a) comprises SEQ ID NO: 3.

19. The fusion polypeptide of claim 17 wherein (b) comprises SEQ ID NO: 5 and SEQ ID NO: 3.

20. The fusion polypeptide of claim 17 wherein (c) comprises SEQ ID NO: 9.

21. The fusion polypeptide of claim 17 wherein (a) and (b) comprise SEQ ID NO: 23.

22. The fusion polypeptide of claim 17 wherein (c) comprises SEQ ID NO: 25.

23. A fusion polypeptide of claim 17 consisting of a polypeptide or functional fragment or variant thereof comprising at least two copies of an amino acid sequence comprising 95% sequence identity to SEQ ID NO: 3 and at least one copy of an amino acid sequence comprising 95% sequence identity to SEQ ID NO: 9.

24. (canceled)

25. (canceled)

26. The fusion polypeptide of claim 23 wherein the at least two copies of SEQ ID NO: 3 form an RBD dimer.

27. The fusion polypeptide of claim 23 wherein the RBD dimer is positioned between the N-terminal of the polypeptide and SEQ ID NO: 9.

28. The fusion polypeptide of claim 23 comprising at least two copies of SEQ ID NO: 23 and at least one copy of SEQ ID NO: 25.

29. (canceled)

30. The fusion polypeptide of claim 23 wherein the RBD dimer is positioned between the N-terminal of the polypeptide and SEQ ID NO: 25.

31. A fusion polypeptide or functional fragment or variant thereof comprising at least 95% sequence identity to SEQ ID NO: 1.

32-34. (canceled)

35. A fusion polypeptide or a functional fragment or variant thereof comprising the amino acid sequences of amino acid positions 1-210, 214-432 and 437 to 715 of SEQ ID NO: 1 or a functional fragment or variant thereof.

36. A fusion polypeptide or a functional fragment or variant thereof comprising the amino acid sequences of amino acid positions 1-219 and 442 to 717 of SEQ ID NO: 21 or a functional fragment or variant thereof.

37. A polynucleotide encoding a fusion polypeptide of claim 1.

38. A pharmaceutical composition comprising the fusion polypeptide of claim 1, and a pharmaceutically acceptable carrier.

39. The pharmaceutical composition of claim 38 that is a vaccine.

40-44. (canceled)


SEQUENCE LISTING (40 sequences)

SEQ ID NO: 1 - protein, 715 aa, Synthetic construct
RFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKGSGRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKSGSGSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVD

SEQ ID NO: 2 - DNA, 2145 bp, Synthetic construct
  source 1..2145: mol_type = other DNA
cgctttccgaacattaccaacctgtgcccgtttggcgaagtgtttaacgcgacccgctttgcgagcgtgtatgcgtggaaccgcaaacgcattagcaactgcgtggcggattatagcgtgctgtataacagcgcgagctttagcacctttaaatgctatggcgtgagcccgaccaaactgaacgatctgtgctttaccaacgtgtatgcggatagctttgtgattcgcggcgatgaagtgcgccagattgcgccgggccagaccggcaaaattgcggattataactataaactgccggatgattttaccggctgcgtgattgcgtggaacagcaacaacctggatagcaaagtgggcggcaactataactatctgtatcgcctgtttcgcaaaagcaacctgaaaccgtttgaacgcgatattagcaccgaaatttatcaggcgggcagcaccccgtgcaacggcgtggaaggctttaactgctattttccgctgcagagctatggctttcagccgaccaacggcgtgggctatcagccgtatcgcgtggtggtgctgagctttgaactgctgcatgcgccggcgaccgtgtgcggcccgaaaaaaagcaccaacctggtgaaaaacaaaggcagcggccgcgtgcagccgaccgaaagcattgtgcgctttccgaacattaccaacctgtgcccgtttggcgaagtgtttaacgcgacccgctttgcgagcgtgtatgcgtggaaccgcaaacgcattagcaactgcgtggcggattatagcgtgctgtataacagcgcgagctttagcacctttaaatgctatggcgtgagcccgaccaaactgaacgatctgtgctttaccaacgtgtatgcggatagctttgtgattcgcggcgatgaagtgcgccagattgcgccgggccagaccggcaaaattgcggattataactataaactgccggatgattttaccggctgcgtgattgcgtggaacagcaacaacctggatagcaaagtgggcggcaactataactatctgtatcgcctgtttcgcaaaagcaacctgaaaccgtttgaacgcgatattagcaccgaaatttatcaggcgggcagcaccccgtgcaacggcgtggaaggctttaactgctattttccgctgcagagctatggctttcagccgaccaacggcgtgggctatcagccgtatcgcgtggtggtgctgagctttgaactgctgcatgcgccggcgaccgtgtgcggcccgaaaaaaagcaccaacctggtgaaaaacaaaagcggcagcggcagcagccagtgcgtgaacctgaccacccgcacccagctgccgccggcgtataccaacagctttacccgcggcgtgtattatccggataaagtgtttcgcagcagcgtgctgcatagcacccaggatctgtttctgccgttttttagcaacgtgacctggtttcatgcgattcatgtgagcggcaccaacggcaccaaacgctttgataacccggtgctgccgtttaacgatggcgtgtattttgcgagcaccgaaaaaagcaacattattcgcggctggatttttggcaccaccctggatagcaaaacccagagcctgctgattgtgaacaacgcgaccaacgtggtgattaaagtgtgcgaatttcagttttgcaacgatccgtttctgggcgtgtattatcataaaaacaacaaaagctggatggaaagcgaatttcgcgtgtatagcagcgcgaacaactgcacctttgaatatgtgagccagccgtttctgatggatctggaaggcaaacagggcaactttaaaaacctgcgcgaatttgtgtttaaaaacattgatggctattttaaaatttatagcaaacataccccgattaacctggtgcgcgatctgccgcagggctttagcgcgctggaaccgctggtggatctgccgattggcattaacattacccgctttcagaccctgctggcgctgcatcgcagctatctgaccccgggcgatagcagcagcggctggaccgcgggcgcggcggcgtattatgtgggctatctgcagccgcgcacctttctgctgaaatataacgaaaacggcaccattaccgatgcggtggat

SEQ ID NO: 3 - protein, 210 aa, Synthetic construct
RFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNK

SEQ ID NO: 4 - DNA, 630 bp, Synthetic construct
  source 1..630: mol_type = other DNA
aggttccccaacatcaccaacctgtgccccttcggcgaggtgttcaacgccaccaggttcgccagcgtgtacgcctggaacaggaagaggatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcaggggcgacgaggtgaggcagatcgcccccggccagaccggcaagatcgccgactacaactacaagctgcccgacgacttcaccggctgcgtgatcgcctggaacagcaacaacctggacagcaaggtgggcggcaactacaactacctgtacaggctgttcaggaagagcaacctgaagcccttcgagagggacatcagcaccgagatctaccaggccggcagcaccccctgcaacggcgtggagggcttcaactgctacttccccctgcagagctacggcttccagcccaccaacggcgtgggctaccagccctacagggtggtggtgctgagcttcgagctgctgcacgcccccgccaccgtgtgcggccccaagaagagcaccaacctggtgaagaacaag

SEQ ID NO: 5 - protein, 219 aa, Synthetic construct
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNK

SEQ ID NO: 6 - DNA, 657 bp, Synthetic construct
  source 1..657: mol_type = other DNA
agggtgcagcccaccgagagcatcgtgaggttccccaacatcaccaacctgtgccccttcggcgaggtgttcaacgccaccaggttcgccagcgtgtacgcctggaacaggaagaggatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcaggggcgacgaggtgaggcagatcgcccccggccagaccggcaagatcgccgactacaactacaagctgcccgacgacttcaccggctgcgtgatcgcctggaacagcaacaacctggacagcaaggtgggcggcaactacaactacctgtacaggctgttcaggaagagcaacctgaagcccttcgagagggacatcagcaccgagatctaccaggccggcagcaccccctgcaacggcgtggagggcttcaactgctacttccccctgcagagctacggcttccagcccaccaacggcgtgggctaccagccctacagggtggtggtgctgagcttcgagctgctgcacgcccccgccaccgtgtgcggccccaagaagagcaccaacctggtgaagaacaag

SEQ ID NO: 7 - protein, 4 aa, Synthetic construct
SGSG

SEQ ID NO: 8 - DNA, 12 bp, Synthetic construct
  source 1..12: mol_type = other DNA
agcggcagcggc

SEQ ID NO: 9 - protein, 278 aa, Synthetic construct
SQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVD

SEQ ID NO: 10 - DNA, 834 bp, Synthetic construct
  source 1..834: mol_type = other DNA
agccagtgcgtgaacctgaccaccaggacccagctgccccccgcctacaccaacagcttcaccaggggcgtgtactaccccgacaaggtgttcaggagcagcgtgctgcacagcacccaggacctgttcctgcccttcttcagcaacgtgacctggttccacgccatccacgtgagcggcaccaacggcaccaagaggttcgacaaccccgtgctgcccttcaacgacggcgtgtacttcgccagcaccgagaagagcaacatcatcaggggctggatcttcggcaccaccctggacagcaagacccagagcctgctgatcgtgaacaacgccaccaacgtggtgatcaaggtgtgcgagttccagttctgcaacgaccccttcctgggcgtgtactaccacaagaacaacaagagctggatggagagcgagttcagggtgtacagcagcgccaacaactgcaccttcgagtacgtgagccagcccttcctgatggacctggagggcaagcagggcaacttcaagaacctgagggagttcgtgttcaagaacatcgacggctacttcaagatctacagcaagcacacccccatcaacctggtgagggacctgccccagggcttcagcgccctggagcccctggtggacctgcccatcggcatcaacatcaccaggttccagaccctgctggccctgcacaggagctacctgacccccggcgacagcagcagcggctggaccgccggcgccgccgcctactacgtgggctacctgcagcccaggaccttcctgctgaagtacaacgagaacggcaccatcaccgacgccgtggac

SEQ ID NO: 11 - protein, 432 aa, Synthetic construct
RFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKGSGRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNK

SEQ ID NO: 12 - DNA, 1296 bp, Synthetic construct
  source 1..1296: mol_type = other DNA
aggttccccaacatcaccaacctgtgccccttcggcgaggtgttcaacgccaccaggttcgccagcgtgtacgcctggaacaggaagaggatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcaggggcgacgaggtgaggcagatcgcccccggccagaccggcaagatcgccgactacaactacaagctgcccgacgacttcaccggctgcgtgatcgcctggaacagcaacaacctggacagcaaggtgggcggcaactacaactacctgtacaggctgttcaggaagagcaacctgaagcccttcgagagggacatcagcaccgagatctaccaggccggcagcaccccctgcaacggcgtggagggcttcaactgctacttccccctgcagagctacggcttccagcccaccaacggcgtgggctaccagccctacagggtggtggtgctgagcttcgagctgctgcacgcccccgccaccgtgtgcggccccaagaagagcaccaacctggtgaagaacaagggcagcggcagggtgcagcccaccgagagcatcgtgaggttccccaacatcaccaacctgtgccccttcggcgaggtgttcaacgccaccaggttcgccagcgtgtacgcctggaacaggaagaggatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcaggggcgacgaggtgaggcagatcgcccccggccagaccggcaagatcgccgactacaactacaagctgcccgacgacttcaccggctgcgtgatcgcctggaacagcaacaacctggacagcaaggtgggcggcaactacaactacctgtacaggctgttcaggaagagcaacctgaagcccttcgagagggacatcagcaccgagatctaccaggccggcagcaccccctgcaacggcgtggagggcttcaactgctacttccccctgcagagctacggcttccagcccaccaacggcgtgggctaccagccctacagggtggtggtgctgagcttcgagctgctgcacgcccccgccaccgtgtgcggccccaagaagagcaccaacctggtgaagaacaag

SEQ ID NO: 13 - protein, 1010 aa, Synthetic construct
MDSKGSSQKGSRLLLLLVVSNLLLCQGVVSDYKDDDDKAAALEVLFQGPRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKGSGRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKSGSGSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDALEVLFQGPDPDPEEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - DNA, 3030 bp, Synthetic construct
  source 1..3030: mol_type = other DNA
atggacagcaagggcagcagccagaagggcagcaggctgctgctgctgctggtggtgagcaacctgctgctgtgccagggcgtggtgagcgactacaaggacgacgacgacaaggccgccgccctggaggtgctgttccagggccccaggttccccaacatcaccaacctgtgccccttcggcgaggtgttcaacgccaccaggttcgccagcgtgtacgcctggaacaggaagaggatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcaggggcgacgaggtgaggcagatcgcccccggccagaccggcaagatcgccgactacaactacaagctgcccgacgacttcaccggctgcgtgatcgcctggaacagcaacaacctggacagcaaggtgggcggcaactacaactacctgtacaggctgttcaggaagagcaacctgaagcccttcgagagggacatcagcaccgagatctaccaggccggcagcaccccctgcaacggcgtggagggcttcaactgctacttccccctgcagagctacggcttccagcccaccaacggcgtgggctaccagccctacagggtggtggtgctgagcttcgagctgctgcacgcccccgccaccgtgtgcggccccaagaagagcaccaacctggtgaagaacaagggcagcggcagggtgcagcccaccgagagcatcgtgaggttccccaacatcaccaacctgtgccccttcggcgaggtgttcaacgccaccaggttcgccagcgtgtacgcctggaacaggaagaggatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcaggggcgacgaggtgaggcagatcgcccccggccagaccggcaagatcgccgactacaactacaagctgcccgacgacttcaccggctgcgtgatcgcctggaacagcaacaacctggacagcaaggtgggcggcaactacaactacctgtacaggctgttcaggaagagcaacctgaagcccttcgagagggacatcagcaccgagatctaccaggccggcagcaccccctgcaacggcgtggagggcttcaactgctacttccccctgcagagctacggcttccagcccaccaacggcgtgggctaccagccctacagggtggtggtgctgagcttcgagctgctgcacgcccccgccaccgtgtgcggccccaagaagagcaccaacctggtgaagaacaagagcggcagcggcagcagccagtgcgtgaacctgaccaccaggacccagctgccccccgcctacaccaacagcttcaccaggggcgtgtactaccccgacaaggtgttcaggagcagcgtgctgcacagcacccaggacctgttcctgcccttcttcagcaacgtgacctggttccacgccatccacgtgagcggcaccaacggcaccaagaggttcgacaaccccgtgctgcccttcaacgacggcgtgtacttcgccagcaccgagaagagcaacatcatcaggggctggatcttcggcaccaccctggacagcaagacccagagcctgctgatcgtgaacaacgccaccaacgtggtgatcaaggtgtgcgagttccagttctgcaacgaccccttcctgggcgtgtactaccacaagaacaacaagagctggatggagagcgagttcagggtgtacagcagcgccaacaactgcaccttcgagtacgtgagccagcccttcctgatggacctggagggcaagcagggcaacttcaagaacctgagggagttcgtgttcaagaacatcgacggctacttcaagatctacagcaagcacacccccatcaacctggtgagggacctgccccagggcttcagcgccctggagcccctggtggacctgcccatcggcatcaacatcaccaggttccagaccctgctggccctgcacaggagctacctgacccccggcgacagcagcagcggctggaccgccggcgccgccgcctactacgtgggctacctgcagcccaggaccttcctgctgaagtacaacgagaacggcaccatcaccgacgccgtggacgccctggaggtgctgttccagggccccgaccccgaccccgaggagcccaagagctgcgacaagacccacacctgccccccctgccccgcccccgagctgctgggcggccccagcgtgttcctgttcccccccaagcccaaggacaccctgatgatcagcaggacccccgaggtgacctgcgtggtggtggacgtgagccacgaggaccccgaggtgaagttcaactggtacgtggacggcgtggaggtgcacaacgccaagaccaagcccagggaggagcagtacaacagcacctacagggtggtgagcgtgctgaccgtgctgcaccaggactggctgaacggcaaggagtacaagtgcaaggtgagcaacaaggccctgcccgcccccatcgagaagaccatcagcaaggccaagggccagcccagggagccccaggtgtacaccctgccccccagcagggacgagctgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagcaggtggcagcagggcaacgtgttcagctgcagcgtgatgcacgaggccctgcacaaccactacacccagaagagcctgagcctgagccccggcaag

SEQ ID NO: 15 - protein, 969 aa, Synthetic construct
MDSKGSSQKGSRLLLLLVVSNLLLCQGVVSDYKDDDDKAAAGSGSLEVLFQGPSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPALEVLFQGPDPDPEEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - DNA, 2907 bp, Synthetic construct
  source 1..2907: mol_type = other DNA
atggacagcaagggcagcagccagaagggcagcaggctgctgctgctgctggtggtgagcaacctgctgctgtgccagggcgtggtgagcgactacaaggacgacgacgacaaggccgccgccggcagcggcagcctggaggtgctgttccagggccccagcagccagtgcgtgaacctgaccaccaggacccagctgccccccgcctacaccaacagcttcaccaggggcgtgtactaccccgacaaggtgttcaggagcagcgtgctgcacagcacccaggacctgttcctgcccttcttcagcaacgtgacctggttccacgccatccacgtgagcggcaccaacggcaccaagaggttcgacaaccccgtgctgcccttcaacgacggcgtgtacttcgccagcaccgagaagagcaacatcatcaggggctggatcttcggcaccaccctggacagcaagacccagagcctgctgatcgtgaacaacgccaccaacgtggtgatcaaggtgtgcgagttccagttctgcaacgaccccttcctgggcgtgtactaccacaagaacaacaagagctggatggagagcgagttcagggtgtacagcagcgccaacaactgcaccttcgagtacgtgagccagcccttcctgatggacctggagggcaagcagggcaacttcaagaacctgagggagttcgtgttcaagaacatcgacggctacttcaagatctacagcaagcacacccccatcaacctggtgagggacctgccccagggcttcagcgccctggagcccctggtggacctgcccatcggcatcaacatcaccaggttccagaccctgctggccctgcacaggagctacctgacccccggcgacagcagcagcggctggaccgccggcgccgccgcctactacgtgggctacctgcagcccaggaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagggtgcagcccaccgagagcatcgtgaggttccccaacatcaccaacctgtgccccttcggcgaggtgttcaacgccaccaggttcgccagcgtgtacgcctggaacaggaagaggatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcaggggcgacgaggtgaggcagatcgcccccggccagaccggcaagatcgccgactacaactacaagctgcccgacgacttcaccggctgcgtgatcgcctggaacagcaacaacctggacagcaaggtgggcggcaactacaactacctgtacaggctgttcaggaagagcaacctgaagcccttcgagagggacatcagcaccgagatctaccaggccggcagcaccccctgcaacggcgtggagggcttcaactgctacttccccctgcagagctacggcttccagcccaccaacggcgtgggctaccagccctacagggtggtggtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctgcccttccagcagttcggcagggacatcgccgacaccaccgacgccgtgagggacccccagaccctggagatcctggacatcaccccctgcagcttcggcggcgtgagcgtgatcacccccggcaccaacaccagcaaccaggtggccgtgctgtaccaggacgtgaactgcaccgaggtgcccgtggccatccacgccgaccagctgacccccacctggagggtgtacagcaccggcagcaacgtgttccagaccagggccggctgcctgatcggcgccgagcacgtgaacaacagctacgagtgcgacatccccatcggcgccggcatctgcgccagctaccagacccagaccaacagccccgccctggaggtgctgttccagggccccgaccccgaccccgaggagcccaagagctgcgacaagacccacacctgccccccctgccccgcccccgagctgctgggcggccccagcgtgttcctgttcccccccaagcccaaggacaccctgatgatcagcaggacccccgaggtgacctgcgtggtggtggacgtgagccacgaggaccccgaggtgaagttcaactggtacgtggacggcgtggaggtgcacaacgccaagaccaagcccagggaggagcagtacaacagcacctacagggtggtgagcgtgctgaccgtgctgcaccaggactggctgaacggcaaggagtacaagtgcaaggtgagcaacaaggccctgcccgcccccatcgagaagaccatcagcaaggccaagggccagcccagggagccccaggtgtacaccctgccccccagcagggacgagctgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagcaggtggcagcagggcaacgtgttcagctgcagcgtgatgcacgaggccctgcacaaccactacacccagaagagcctgagcctgagccccggcaag

SEQ ID NO: 17 - protein, 788 aa, Synthetic construct
MDSKGSSQKGSRLLLLLVVSNLLLCQGVVSDYKDDDDKAAALEVLFQGPSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDGSGSRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNSALEVLFQGPDPDPEEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - DNA, 2364 bp, Synthetic construct
  source 1..2364: mol_type = other DNA
atggacagcaagggcagcagccagaagggcagcaggctgctgctgctgctggtggtgagcaacctgctgctgtgccagggcgtggtgagcgactacaaggacgacgacgacaaggccgccgccctggaggtgctgttccagggccccagcagccagtgcgtgaacctgaccaccaggacccagctgccccccgcctacaccaacagcttcaccaggggcgtgtactaccccgacaaggtgttcaggagcagcgtgctgcacagcacccaggacctgttcctgcccttcttcagcaacgtgacctggttccacgccatccacgtgagcggcaccaacggcaccaagaggttcgacaaccccgtgctgcccttcaacgacggcgtgtacttcgccagcaccgagaagagcaacatcatcaggggctggatcttcggcaccaccctggacagcaagacccagagcctgctgatcgtgaacaacgccaccaacgtggtgatcaaggtgtgcgagttccagttctgcaacgaccccttcctgggcgtgtactaccacaagaacaacaagagctggatggagagcgagttcagggtgtacagcagcgccaacaactgcaccttcgagtacgtgagccagcccttcctgatggacctggagggcaagcagggcaacttcaagaacctgagggagttcgtgttcaagaacatcgacggctacttcaagatctacagcaagcacacccccatcaacctggtgagggacctgccccagggcttcagcgccctggagcccctggtggacctgcccatcggcatcaacatcaccaggttccagaccctgctggccctgcacaggagctacctgacccccggcgacagcagcagcggctggaccgccggcgccgccgcctactacgtgggctacctgcagcccaggaccttcctgctgaagtacaacgagaacggcaccatcaccgacgccgtggacggcagcggcagcaggttccccaacatcaccaacctgtgccccttcggcgaggtgttcaacgccaccaggttcgccagcgtgtacgcctggaacaggaagaggatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcaggggcgacgaggtgaggcagatcgcccccggccagaccggcaagatcgccgactacaactacaagctgcccgacgacttcaccggctgcgtgatcgcctggaacagcaacaacctggacagcaaggtgggcggcaactacaactacctgtacaggctgttcaggaagagcaacctgaagcccttcgagagggacatcagcaccgagatctaccaggccggcagcaccccctgcaacggcgtggagggcttcaactgctacttccccctgcagagctacggcttccagcccaccaacggcgtgggctaccagccctacagggtggtggtgctgagcttcgagctgctgcacgcccccgccaccgtgtgcggccccaagaagagcaccaacctggtgaagaacagcgccctggaggtgctgttccagggccccgaccccgaccccgaggagcccaagagctgcgacaagacccacacctgccccccctgccccgcccccgagctgctgggcggccccagcgtgttcctgttcccccccaagcccaaggacaccctgatgatcagcaggacccccgaggtgacctgcgtggtggtggacgtgagccacgaggaccccgaggtgaagttcaactggtacgtggacggcgtggaggtgcacaacgccaagaccaagcccagggaggagcagtacaacagcacctacagggtggtgagcgtgctgaccgtgctgcaccaggactggctgaacggcaaggagtacaagtgcaaggtgagcaacaaggccctgcccgcccccatcgagaagaccatcagcaaggccaagggccagcccagggagccccaggtgtacaccctgccccccagcagggacgagctgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagcaggtggcagcagggcaacgtgttcagctgcagcgtgatgcacgaggccctgcacaaccactacacccagaagagcctgagcctgagccccggcaag

SEQ ID NO: 19 - protein, 1273 aa, Severe acute respiratory syndrome-related coronavirus
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 20 - DNA, 3819 bp, Synthetic construct
  source 1..3819: mol_type = other DNA
atgttcgtgttcctggtgctgctgcccctggtgagcagccagtgcgtgaacctgaccaccaggacccagctgccccccgcctacaccaacagcttcaccaggggcgtgtactaccccgacaaggtgttcaggagcagcgtgctgcacagcacccaggacctgttcctgcccttcttcagcaacgtgacctggttccacgccatccacgtgagcggcaccaacggcaccaagaggttcgacaaccccgtgctgcccttcaacgacggcgtgtacttcgccagcaccgagaagagcaacatcatcaggggctggatcttcggcaccaccctggacagcaagacccagagcctgctgatcgtgaacaacgccaccaacgtggtgatcaaggtgtgcgagttccagttctgcaacgaccccttcctgggcgtgtactaccacaagaacaacaagagctggatggagagcgagttcagggtgtacagcagcgccaacaactgcaccttcgagtacgtgagccagcccttcctgatggacctggagggcaagcagggcaacttcaagaacctgagggagttcgtgttcaagaacatcgacggctacttcaagatctacagcaagcacacccccatcaacctggtgagggacctgccccagggcttcagcgccctggagcccctggtggacctgcccatcggcatcaacatcaccaggttccagaccctgctggccctgcacaggagctacctgacccccggcgacagcagcagcggctggaccgccggcgccgccgcctactacgtgggctacctgcagcccaggaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagggtgcagcccaccgagagcatcgtgaggttccccaacatcaccaacctgtgccccttcggcgaggtgttcaacgccaccaggttcgccagcgtgtacgcctggaacaggaagaggatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcaggggcgacgaggtgaggcagatcgcccccggccagaccggcaagatcgccgactacaactacaagctgcccgacgacttcaccggctgcgtgatcgcctggaacagcaacaacctggacagcaaggtgggcggcaactacaactacctgtacaggctgttcaggaagagcaacctgaagcccttcgagagggacatcagcaccgagatctaccaggccggcagcaccccctgcaacggcgtggagggcttcaactgctacttccccctgcagagctacggcttccagcccaccaacggcgtgggctaccagccctacagggtggtggtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctgcccttccagcagttcggcagggacatcgccgacaccaccgacgccgtgagggacccccagaccctggagatcctggacatcaccccctgcagcttcggcggcgtgagcgtgatcacccccggcaccaacaccagcaaccaggtggccgtgctgtaccaggacgtgaactgcaccgaggtgcccgtggccatccacgccgaccagctgacccccacctggagggtgtacagcaccggcagcaacgtgttccagaccagggccggctgcctgatcggcgccgagcacgtgaacaacagctacgagtgcgacatccccatcggcgccggcatctgcgccagctaccagacccagaccaacagccccaggagggccaggagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaacagggccctgaccggcatcgccgtggagcaggacaagaacacccaggaggtgttcgcccaggtgaagcagatctacaagaccccccccatcaaggacttcggcggcttcaacttcagccagatcctgcccgaccccagcaagcccagcaagaggagcttcatcgaggacctgctgttcaacaaggtgaccctggccgacgccggcttcatcaagcagtacggcgactgcctgggcgacatcgccgccagggacctgatctgcgcccagaagttcaacggcctgaccgtgctgccccccctgctgaccgacgagatgatcgcccagtacaccagcgccctgctggccggcaccatcaccagcggctggaccttcggcgccggcgccgccctgcagatccccttcgccatgcagatggcctacaggttcaacggcatcggcgtgacccagaacgtgctgtacgagaaccagaagctgatcgccaaccagttcaacagcgccatcggcaagatccaggacagcctgagcagcaccgccagcgccctgggcaagctgcaggacgtggtgaaccagaacgcccaggccctgaacaccctggtgaagcagctgagcagcaacttcggcgccatcagcagcgtgctgaacgacatcctgagcaggctggacaaggtggaggccgaggtgcagatcgacaggctgatcaccggcaggctgcagagcctgcagacctacgtgacccagcagctgatcagggccgccgagatcagggccagcgccaacctggccgccaccaagatgagcgagtgcgtgctgggccagagcaagagggtggacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttccccagggagggcgtgttcgtgagcaacggcacccactggttcgtgacccagaggaacttctacgagccccagatcatcaccaccgacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcgtggtgaacatccagaaggagatcgacaggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacacc

SEQ ID NO: 21 - protein, 717 aa, Synthetic construct
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKGRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKGSSQCVNLRTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMEFGVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVD

SEQ ID NO: 22 - DNA, 2151 bp, Synthetic construct
  source 1..2151: mol_type = other DNA
agggtgcagcccaccgagagcatcgtgaggttccccaacatcaccaacctgtgccccttcggcgaggtgttcaacgccaccaggttcgccagcgtgtacgcctggaacaggaagaggatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcaggggcgacgaggtgaggcagatcgcccccggccagaccggcaagatcgccgactacaactacaagctgcccgacgacttcaccggctgcgtgatcgcctggaacagcaacaacctggacagcaaggtgggcggcaactacaactacaggtacaggctgttcaggaagagcaacctgaagcccttcgagagggacatcagcaccgagatctaccaggccggcagcaagccctgcaacggcgtggagggcttcaactgctacttccccctgcagagctacggcttccagcccaccaacggcgtgggctaccagccctacagggtggtggtgctgagcttcgagctgctgcacgcccccgccaccgtgtgcggccccaagaagagcaccaacctggtgaagaacaagggcagggtgcagcccaccgagagcatcgtgaggttccccaacatcaccaacctgtgccccttcggcgaggtgttcaacgccaccaggttcgccagcgtgtacgcctggaacaggaagaggatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcaggggcgacgaggtgaggcagatcgcccccggccagaccggcaagatcgccgactacaactacaagctgcccgacgacttcaccggctgcgtgatcgcctggaacagcaacaacctggacagcaaggtgggcggcaactacaactacaggtacaggctgttcaggaagagcaacctgaagcccttcgagagggacatcagcaccgagatctaccaggccggcagcaagccctgcaacggcgtggagggcttcaactgctacttccccctgcagagctacggcttccagcccaccaacggcgtgggctaccagccctacagggtggtggtgctgagcttcgagctgctgcacgcccccgccaccgtgtgcggccccaagaagagcaccaacctggtgaagaacaagggcagcagccagtgcgtgaacctgaggaccaggacccagctgccccccgcctacaccaacagcttcaccaggggcgtgtactaccccgacaaggtgttcaggagcagcgtgctgcacagcacccaggacctgttcctgcccttcttcagcaacgtgacctggttccacgccatccacgtgagcggcaccaacggcaccaagaggttcgacaaccccgtgctgcccttcaacgacggcgtgtacttcgccagcaccgagaagagcaacatcatcaggggctggatcttcggcaccaccctggacagcaagacccagagcctgctgatcgtgaacaacgccaccaacgtggtgatcaaggtgtgcgagttccagttctgcaacgaccccttcctggacgtgtactaccacaagaacaacaagagctggatggagttcggcgtgtacagcagcgccaacaactgcaccttcgagtacgtgagccagcccttcctgatggacctggagggcaagcagggcaacttcaagaacctgagggagttcgtgttcaagaacatcgacggctacttcaagatctacagcaagcacacccccatcaacctggtgagggacctgccccagggcttcagcgccctggagcccctggtggacctgcccatcggcatcaacatcaccaggttccagaccctgctggccctgcacaggagctacctgacccccggcgacagcagcagcggctggaccgccggcgccgccgcctactacgtgggctacctgcagcccaggaccttcctgctgaagtacaacgagaacggcaccatcaccgacgccgtggac

SEQ ID NO: 23 - protein, 219 aa, Synthetic construct
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNK

SEQ ID NO: 24 - DNA, 657 bp, Synthetic construct
  source 1..657: mol_type = other DNA
agggtgcagcccaccgagagcatcgtgaggttccccaacatcaccaacctgtgccccttcggcgaggtgttcaacgccaccaggttcgccagcgtgtacgcctggaacaggaagaggatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcaggggcgacgaggtgaggcagatcgcccccggccagaccggcaagatcgccgactacaactacaagctgcccgacgacttcaccggctgcgtgatcgcctggaacagcaacaacctggacagcaaggtgggcggcaactacaactacaggtacaggctgttcaggaagagcaacctgaagcccttcgagagggacatcagcaccgagatctaccaggccggcagcaagccctgcaacggcgtggagggcttcaactgctacttccccctgcagagctacggcttccagcccaccaacggcgtgggctaccagccctacagggtggtggtgctgagcttcgagctgctgcacgcccccgccaccgtgtgcggccccaagaagagcaccaacctggtgaagaacaag

SEQ ID NO: 25 - protein, 276 aa, Synthetic construct
SQCVNLRTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMEFGVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVD

SEQ ID NO: 26 - DNA, 828 bp, Synthetic construct
  source 1..828: mol_type = other DNA
agccagtgcgtgaacctgaggaccaggacccagctgccccccgcctacaccaacagcttcaccaggggcgtgtactaccccgacaaggtgttcaggagcagcgtgctgcacagcacccaggacctgttcctgcccttcttcagcaacgtgacctggttccacgccatccacgtgagcggcaccaacggcaccaagaggttcgacaaccccgtgctgcccttcaacgacggcgtgtacttcgccagcaccgagaagagcaacatcatcaggggctggatcttcggcaccaccctggacagcaagacccagagcctgctgatcgtgaacaacgccaccaacgtggtgatcaaggtgtgcgagttccagttctgcaacgaccccttcctggacgtgtactaccacaagaacaacaagagctggatggagttcggcgtgtacagcagcgccaacaactgcaccttcgagtacgtgagccagcccttcctgatggacctggagggcaagcagggcaacttcaagaacctgagggagttcgtgttcaagaacatcgacggctacttcaagatctacagcaagcacacccccatcaacctggtgagggacctgccccagggcttcagcgccctggagcccctggtggacctgcccatcggcatcaacatcaccaggttccagaccctgctggccctgcacaggagctacctgacccccggcgacagcagcagcggctggaccgccggcgccgccgcctactacgtgggctacctgcagcccaggaccttcctgctgaagtacaacgagaacggcaccatcaccgacgccgtggac

SEQ ID NO: 27 - protein, 439 aa, Synthetic construct
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKGRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNK

SEQ ID NO: 28 - DNA, 1317 bp, Synthetic construct
  source 1..1317: mol_type = other DNA
agggtgcagcccaccgagagcatcgtgaggttccccaacatcaccaacctgtgccccttcggcgaggtgttcaacgccaccaggttcgccagcgtgtacgcctggaacaggaagaggatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcaggggcgacgaggtgaggcagatcgcccccggccagaccggcaagatcgccgactacaactacaagctgcccgacgacttcaccggctgcgtgatcgcctggaacagcaacaacctggacagcaaggtgggcggcaactacaactacaggtacaggctgttcaggaagagcaacctgaagcccttcgagagggacatcagcaccgagatctaccaggccggcagcaagccctgcaacggcgtggagggcttcaactgctacttccccctgcagagctacggcttccagcccaccaacggcgtgggctaccagccctacagggtggtggtgctgagcttcgagctgctgcacgcccccgccaccgtgtgcggccccaagaagagcaccaacctggtgaagaacaagggcagggtgcagcccaccgagagcatcgtgaggttccccaacatcaccaacctgtgccccttcggcgaggtgttcaacgccaccaggttcgccagcgtgtacgcctggaacaggaagaggatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcaggggcgacgaggtgaggcagatcgcccccggccagaccggcaagatcgccgactacaactacaagctgcccgacgacttcaccggctgcgtgatcgcctggaacagcaacaacctggacagcaaggtgggcggcaactacaactacaggtacaggctgttcaggaagagcaacctgaagcccttcgagagggacatcagcaccgagatctaccaggccggcagcaagccctgcaacggcgtggagggcttcaactgctacttccccctgcagagctacggcttccagcccaccaacggcgtgggctaccagccctacagggtggtggtgctgagcttcgagctgctgcacgcccccgccaccgtgtgcggccccaagaagagcaccaacctggtgaagaacaag

SEQ ID NO: 29 - protein, 1012 aa, Synthetic construct
MDSKGSSQKGSRLLLLLVVSNLLLCQGVVSDYKDDDDKAAALEVLFQGPRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKGRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKGSSQCVNLRTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMEFGVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDALEVLFQGPDPDPEEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - DNA, 3039 bp, Synthetic construct
  source 1..3039: mol_type = other DNA
atggacagcaagggcagcagccagaagggcagcaggctgctgctgctgctggtggtgagcaacctgctgctgtgccagggcgtggtgagcgactacaaggacgacgacgacaaggccgccgccctggaggtgctgttccagggccccagggtgcagcccaccgagagcatcgtgaggttccccaacatcaccaacctgtgccccttcggcgaggtgttcaacgccaccaggttcgccagcgtgtacgcctggaacaggaagaggatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcaggggcgacgaggtgaggcagatcgcccccggccagaccggcaagatcgccgactacaactacaagctgcccgacgacttcaccggctgcgtgatcgcctggaacagcaacaacctggacagcaaggtgggcggcaactacaactacaggtacaggctgttcaggaagagcaacctgaagcccttcgagagggacatcagcaccgagatctaccaggccggcagcaagccctgcaacggcgtggagggcttcaactgctacttccccctgcagagctacggcttccagcccaccaacggcgtgggctaccagccctacagggtggtggtgctgagcttcgagctgctgcacgcccccgccaccgtgtgcggccccaagaagagcaccaacctggtgaagaacaagggcagggtgcagcccaccgagagcatcgtgaggttccccaacatcaccaacctgtgccccttcggcgaggtgttcaacgccaccaggttcgccagcgtgtacgcctggaacaggaagaggatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcaggggcgacgaggtgaggcagatcgcccccggccagaccggcaagatcgccgactacaactacaagctgcccgacgacttcaccggctgcgtgatcgcctggaacagcaacaacctggacagcaaggtgggcggcaactacaactacaggtacaggctgttcaggaagagcaacctgaagcccttcgagagggacatcagcaccgagatctaccaggccggcagcaagccctgcaacggcgtggagggcttcaactgctacttccccctgcagagctacggcttccagcccaccaacggcgtgggctaccagccctacagggtggtggtgctgagcttcgagctgctgcacgcccccgccaccgtgtgcggccccaagaagagcaccaacctggtgaagaacaagggcagcagccagtgcgtgaacctgaggaccaggacccagctgccccccgcctacaccaacagcttcaccaggggcgtgtactaccccgacaaggtgttcaggagcagcgtgctgcacagcacccaggacctgttcctgcccttcttcagcaacgtgacctggttccacgccatccacgtgagcggcaccaacggcaccaagaggttcgacaaccccgtgctgcccttcaacgacggcgtgtacttcgccagcaccgagaagagcaacatcatcaggggctggatcttcggcaccaccctggacagcaagacccagagcctgctgatcgtgaacaacgccaccaacgtggtgatcaaggtgtgcgagttccagttctgcaacgaccccttcctggacgtgtactaccacaagaacaacaagagctggatggagttcggcgtgtacagcagcgccaacaactgcaccttcgagtacgtgagccagcccttcctgatggacctggagggcaagcagggcaacttcaagaacctgagggagttcgtgttcaagaacatcgacggctacttcaagatctacagcaagcacacccccatcaacctggtgagggacctgccccagggcttcagcgccctggagcccctggtggacctgcccatcggcatcaacatcaccaggttccagaccctgctggccctgcacaggagctacctgacccccggcgacagcagcagcggctggaccgccggcgccgccgcctactacgtgggctacctgcagcccaggaccttcctgctgaagtacaacgagaacggcaccatcaccgacgccgtggacgccctggaggtgctgttccagggccccgaccccgaccccgaggagcccaagagctgcgacaagacccacacctgccccccctgccccgcccccgagctgctgggcggccccagcgtgttcctgttcccccccaagcccaaggacaccctgatgatcagcaggacccccgaggtgacctgcgtggtggtggacgtgagccacgaggaccccgaggtgaagttcaactggtacgtggacggcgtggaggtgcacaacgccaagaccaagcccagggaggagcagtacaacagcacctacagggtggtgagcgtgctgaccgtgctgcaccaggactggctgaacggcaaggagtacaagtgcaaggtgagcaacaaggccctgcccgcccccatcgagaagaccatcagcaaggccaagggccagcccagggagccccaggtgtacaccctgccccccagcagggacgagctgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagcaggtggcagcagggcaacgtgttcagctgcagcgtgatgcacgaggccctgcacaaccactacacccagaagagcctgagcctgagccccggcaagtag

SEQ ID NO: 31 - protein, 715 aa, Synthetic construct; identical to SEQ ID NO: 1

SEQ ID NO: 32 - RNA, 2268 nt, Synthetic construct
  source 1..2268: mol_type = other RNA
atggacagcaaaggttcgtcgcagaaagggtcccgcctgctcctgctgctggtggtgtcaaatctactcttgtgccagggtgtggtctccagagtgcagcctacagagtccatcgtgaggttccccaacatcacaaatctgtgccccttcggcgaggtgttcaatgccacaagattcgccagcgtgtacgcctggaacagaaagagaatcagcaactgcgtggccgactactccgtgctgtacaatagcgccagcttcagcaccttcaagtgctacggcgtgagccccaccaagctgaatgatctgtgcttcaccaatgtgtacgccgattccttcgtgatcagaggcgatgaggtgaggcagatcgcccccggccagaccggaaagatcgccgattacaactacaagctgcccgatgacttcacaggctgtgtgatcgcctggaactccaataacctggattccaaggtgggcggcaactacaattacctgtacagactgttcagaaagtccaatctgaagcccttcgagagggatatcagcacagagatctaccaggccggctccacaccctgcaacggcgtggagggcttcaactgctacttccccctgcagagctacggcttccagcctacaaacggcgtgggctaccagccctacagggtggtggtgctgagcttcgagctgctgcacgcccccgccacagtgtgcggaccaaagaagtccacaaacctggtgaagaacaagggctccggcagagtgcagcctacagagtccatcgtgaggttccccaacatcacaaatctgtgccccttcggcgaggtgttcaatgccacaagattcgccagcgtgtacgcctggaacagaaagagaatcagcaactgcgtggccgactactccgtgctgtacaatagcgccagcttcagcaccttcaagtgctacggcgtgagccccaccaagctgaatgatctgtgcttcaccaatgtgtacgccgattccttcgtgatcagaggcgatgaggtgaggcagatcgcccccggccagaccggaaagatcgccgattacaactacaagctgcccgatgacttcacaggctgtgtgatcgcctggaactccaataacctggattccaaggtgggcggcaactacaattacctgtacagactgttcagaaagtccaatctgaagcccttcgagagggatatcagcacagagatctaccaggccggctccacaccctgcaacggcgtggagggcttcaactgctacttccccctgcagagctacggcttccagcctacaaacggcgtgggctaccagccctacagggtggtggtgctgagcttcgagctgctgcacgcccccgccacagtgtgcggaccaaagaagtccacaaacctggtgaagaacaagtccggcagcggctccagccagtgcgtgaatctgaccaccaggacccagctgccccccgcttacacaaacagcttcaccaggggcgtgtactaccccgataaggtgttcagatccagcgtgctgcactccacacaggatctgttcctgcccttcttcagcaacgtgacctggttccacgccatccacgtgtccggcaccaatggcacaaagaggttcgataaccctgtgctgcccttcaacgacggcgtgtacttcgcctccacagagaagtccaacatcatcagaggctggatcttcggcaccaccctggacagcaaaacacaaagcctgctgatcgtgaataacgccaccaatgtggtgatcaaggtgtgcgagttccagttctgcaacgatcccttcctgggcgtgtactatcacaagaacaataagtcctggatggagagcgagttcagggtgtacagctccgccaacaattgtacattcgagtacgtgtcccagcccttcctgatggatctggagggcaagcagggcaacttcaagaacctgagagagttcgtgttcaagaacatcgatggctacttcaagatctacagcaagcacacccctatcaacctggtgagggatctgcctcagggcttcagcgccctggagcctctggtggacctgcccatcggcatcaatatcaccagattccagaccctgctggccctgcacagaagctacctgacccctggcgattccagcagcggctggaccgccggagccgctgcttactacgtgggctacctgcagcccaggacattcctgctgaagtacaacgagaatggcaccatcaccgatgccgtggacgcttga

SEQ ID NO: 33 - RNA, 657 nt, Synthetic construct
  source 1..657: mol_type = other RNA
agagtgcagcctacagagtccatcgtgaggttccccaacatcacaaatctgtgccccttcggcgaggtgttcaatgccacaagattcgccagcgtgtacgcctggaacagaaagagaatcagcaactgcgtggccgactactccgtgctgtacaatagcgccagcttcagcaccttcaagtgctacggcgtgagccccaccaagctgaatgatctgtgcttcaccaatgtgtacgccgattccttcgtgatcagaggcgatgaggtgaggcagatcgcccccggccagaccggaaagatcgccgattacaactacaagctgcccgatgacttcacaggctgtgtgatcgcctggaactccaataacctggattccaaggtgggcggcaactacaattacctgtacagactgttcagaaagtccaatctgaagcccttcgagagggatatcagcacagagatctaccaggccggctccacaccctgcaacggcgtggagggcttcaactgctacttccccctgcagagctacggcttccagcctacaaacggcgtgggctaccagccctacagggtggtggtgctgagcttcgagctgctgcacgcccccgccacagtgtgcggaccaaagaagtccacaaacctggtgaagaacaag

SEQ ID NO: 34 - RNA, 657 nt, Synthetic construct; identical to SEQ ID NO: 33
  source 1..657: mol_type = other RNA

SEQ ID NO: 35 - RNA, 840 nt, Synthetic construct
  source 1..840: mol_type = other RNA
agccagtgcgtgaatctgaccaccaggacccagctgccccccgcttacacaaacagcttcaccaggggcgtgtactaccccgataaggtgttcagatccagcgtgctgcactccacacaggatctgttcctgcccttcttcagcaacgtgacctggttccacgccatccacgtgtccggcaccaatggcacaaagaggttcgataaccctgtgctgcccttcaacgacggcgtgtacttcgcctccacagagaagtccaacatcatcagaggctggatcttcggcaccaccctggacagcaaaacacaaagcctgctgatcgtgaataacgccaccaatgtggtgatcaaggtgtgcgagttccagttctgcaacgatcccttcctgggcgtgtactatcacaagaacaataagtcctggatggagagcgagttcagggtgtacagctccgccaacaattgtacattcgagtacgtgtcccagcccttcctgatggatctggagggcaagcagggcaacttcaagaacctgagagagttcgtgttcaagaacatcgatggctacttcaagatctacagcaagcacacccctatcaacctggtgagggatctgcctcagggcttcagcgccctggagcctctggtggacctgcccatcggcatcaatatcaccagattccagaccctgctggccctgcacagaagctacctgacccctggcgattccagcagcggctggaccgccggagccgctgcttactacgtgggctacctgcagcccaggacattcctgctgaagtacaacgagaatggcaccatcaccgatgccgtggacgcttga

SEQ ID NO: 36 - RNA, 53 nt, Synthetic construct
  source 1..53: mol_type = other RNA
agaataaactagtattcttctggtccccacagactcagagagaacccgccacc

SEQ ID NO: 37 - RNA, 295 nt, Synthetic construct
  source 1..295: mol_type = other RNA
ctcgagctggtactgcatgcacgcaatgcaagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagcttgc

SEQ ID NO: 38 - protein, 30 aa, Synthetic construct
MDSKGSSQKGSRLLLLLVVSNLLLCQGVVS

SEQ ID NO: 39 - RNA, 90 nt, Synthetic construct
  source 1..90: mol_type = other RNA
atggacagcaaaggttcgtcgcagaaagggtcccgcctgctcctgctgctggtggtgtcaaatctactcttgtgccagggtgtggtctcc

SEQ ID NO: 40 - DNA, 5577 bp, Synthetic construct
  source 1..5577: mol_type = other DNA
gactcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcagaataaactagtattcttctggtccccacagactcagagagaacccgccaccaccggtgccaccatggacagcaaaggttcgtcgcagaaagggtcccgcctgctcctgctgctggtggtgtcaaatctactcttgtgccagggtgtggtctccagagtgcagcctacagagagcatcgtgagattccctaatatcacaaatctgtgccccttcggcgaggtgttcaacgccacaagattcgcctccgtgtacgcctggaatagaaagaggatcagcaattgcgtggccgattactccgtgctgtacaatagcgcctccttctccaccttcaagtgttacggcgtgtcccccacaaagctgaatgacctgtgcttcacaaacgtgtacgccgacagcttcgtgatcaggggcgacgaggtgaggcagatcgcccctggccagacaggcaagatcgccgattacaattacaagctgcccgacgacttcacaggctgcgtgatcgcctggaacagtaataacctggacagcaaggtgggcggcaactacaattacctgtacaggctgttcagaaagtccaacctgaagcccttcgagagagatatctccaccgagatctaccaggccggcagcaccccctgcaacggcgttgagggcttcaactgctacttccctctgcagagctacggcttccagcccaccaacggcgtgggctaccagccctacagagtggtggtgctgtccttcgagctgctgcacgcccctgccacagtgtgtggccccaagaagtccaccaatctggtgaagaataagggctccggcagagtgcagcctacagagagcatcgtgagattccctaatatcacaaatctgtgccccttcggcgaggtgttcaacgccacaagattcgcctccgtgtacgcctggaatagaaagaggatcagcaattgcgtggccgattactccgtgctgtacaatagcgcctccttctccaccttcaagtgttacggcgtgtcccccacaaagctgaatgacctgtgcttcacaaacgtgtacgccgacagcttcgtgatcaggggcgacgaggtgaggcagatcgcccctggccagacaggcaagatcgccgattacaattacaagctgcccgacgacttcacaggctgcgtgatcgcctggaacagtaataacctggacagcaaggtgggcggcaactacaattacctgtacaggctgttcagaaagtccaacctgaagcccttcgagagagatatctccaccgagatctaccaggccggcagcaccccctgcaacggcgttgagggcttcaactgctacttccctctgcagagctacggcttccagcccaccaacggcgtgggctaccagccctacagagtggtggtgctgtccttcgagctgctgcacgcccctgccacagtgtgtggccccaagaagtccaccaatctggtgaagaataagtccggcagcggctccagccagtgcgtgaatctgaccaccaggacccagctgccccccgcttacacaaacagcttcaccaggggcgtgtactaccccgataaggtgttcagatccagcgtgctgcactccacacaggatctgttcctgcccttcttcagcaacgtgacctggttccacgccatccacgtgtccggcaccaatggcacaaagaggttcgataaccctgtgctgcccttcaacgacggcgtgtacttcgcctccacagagaagtccaacatcatcagaggctggatcttcggcaccaccctggacagcaaaacacaaagcctgctgatcgtgaataacgccaccaatgtggtgatcaaggtgtgcgagttccagttctgcaacgatcccttcctgggcgtgtactatcacaagaacaataagtcctggatggagagcgagttcagggtgtacagctccgccaacaattgtacattcgagtacgtgtcccagcccttcctgatggatctggagggcaagcagggcaacttcaagaacctgagagagttcgtgttcaagaacatcgatggctacttcaagatctacagcaagcacacccctatcaacctggtgagggatctgcctcagggcttcagcgccctggagcctctggtggacctgcccatcggcatcaatatcaccagattccagaccctgctggccctgcacagaagctacctgacccctggcgattccagcagcggctggaccgccggagccgctgcttactacgtgggctacctgcagcccaggacattcctgctgaagtacaacgagaatggcaccatcaccgatgccgtggacgcttgagaattcctcgagctggtactgcatgcacgcaatgcaagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagcttgcgcggccgcgtgcagatatccagcacagtgtcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctt